(19) United States
(12) Patent Application Publication
(10) Pub. No.: US 2023/0045737 A1
(43) Pub. Date: Feb. 9, 2023

(54) SUBSTITUTED ISOINDOLINONES AS MODULATORS OF CEREBLON-MEDIATED NEO-SUBSTRATE RECRUITMENT

(71) Applicant: Vividion Therapeutics, Inc., San Diego, CA (US)

(72) Inventors: Robert HUBBARD, San Diego, CA (US); Betty LAM, San Diego, CA (US); Shota KIKUCHI, San Diego, CA (US); Flora HUYNH, San Diego, CA (US); Chung-Mao PAN, San Diego, CA (US); Dan BEISNER, San Diego, CA (US); Eric ALLEN, San Diego, CA (US)

(21) Appl. No.: 17/299,761

(22) PCT Filed: Dec. 5, 2019
(86) PCT No.: PCT/US19/64763

§ 371 Date: Jun. 3, 2021

Related U.S. Application Data
(60) Provisional application No. 62/775,861, filed on Dec. 5, 2018.

(51) Int. Cl. C07D 401/14; C07D 401/04

(52) U.S. Cl. CPC C07D 401/14; C07D 401/04

(57) ABSTRACT

Disclosed herein are isoindolinone compounds and methods for binding cereblon and for modulating cereblon neosubstrates. The isoindolinone compounds can have a structure of Formula (I).

### CROSS-REFERENCE

This application claims the benefit of U.S. Provisional Application No. 62/775,861, filed Dec. 5, 2018, which is incorporated herein by reference in its entirety.

### BACKGROUND OF THE DISCLOSURE

Protein biosynthesis and degradation is a dynamic process which sustains normal cell metabolism. In some instances, production of new proteins modulate proliferation and differentiation of cells and upon completion, these protein are degraded through one of two proteolytic mechanisms, the lysosome degradation system or the ubiquitin proteasome pathway. In some cases, a majority of cellular proteins are degraded by the proteasome pathway, and the process is initiated via tagging of a ubiquitin.

One E3 ligase with therapeutic potential is the von Hippel-Lindau (VHL) tumor suppressor. VHL comprises the substrate recognition subunit/E3 ligase complex VCB, which includes elongins B and C, and a complex including Cullin-2 and Rbx1. The primary substrate of VHL is Hypoxia Inducible Factor 1a (HIF-1a), a transcription factor that upregulates genes such as the pro-angiogenic growth factor VEGF and the red blood cell inducing cytokine erythropoietin in response to low oxygen levels. We generated the first small molecule ligands of Von Hippel Lindau (VHL) to the substrate recognition subunit of the E3 ligase, VCB, an important target in cancer, chronic anemia and ischemia, and obtained crystal structures confirming that the compound mimics the binding mode of the transcription factor HIF-1.alpha., the major substrate of VHL.

Cereblon is a protein that in humans is encoded by the CRBN gene. CRBN orthologs are highly conserved from plants to humans, which underscores its physiological importance. Cereblon forms an E3 ubiquitin ligase complex with damaged DNA binding protein 1 (DDB1), Cullin-4A (CUL4A), and regulator of cullins 1 (ROC1). This complex ubiquitinates a number of other proteins. Through a mechanism which has not been completely elucidated, cereblon ubquitination of target proteins results in increased levels of fibroblast growth factor 8 (FGF8) and fibroblast growth factor 10 (FGF10). FGF8 in turn regulates a number of developmental processes, such as limb and auditory vesicle formation. The net result is that this ubiquitin ligase complex is important for limb outgrowth in embryos. In the absence of cereblon, DDB1 forms a complex with DDB2 that functions as a DNA damage-binding protein.

Thalidomide, which has been approved for the treatment of a number of immunological indications, has also been approved for the treatment of certain neoplastic diseases, including multiple myeloma. In addition to multiple myeloma, thalidomide and several of its analogs are also currently under investigation for use in treating a variety of other types of cancer. While the precise mechanism of thalidomide's anti-tumor activity is still emerging, it is known to inhibit angiogenesis. Recent literature discussing the biology of the imides includes Lu et al Science 343, 305 (2014) and Kronke et al Science 343, 301 (2014).

Significantly, thalidomide and its analogs e.g. pomolinamiode and lenalinomide, are known to bind cereblon. These agents bind to cereblon, altering the specificity of the complex to induce the ubiquitination and degradation of Ikaros (IKZF1) and Aiolos (IKZF3), transcription factors essential for multiple myeloma growth. Indeed, higher expression of cereblon has been linked to an increase in efficacy of imide drugs in the treatment of multiple myeloma.

BRD4 has captured considerable attention from academia and Pharmaceutical industry alike due to its great potential as a novel target in multiple disease settings, particularly in cancer. BRD4 belongs to the bromodomain and extra-terminal domain (BET) family, which is characterized by two bromodomains (BD domain) at the N-terminus and an extraterminal domain (ET domain) at the C-terminus (J. Shi, et al. Molecular cell, 54 (2014) 728-736 and A. C. Belkina, et al., Nat. Rev. Cancer, 12 (2012) 465-477). The two BD domains recognize and interact with acetylated-lysine residues at the N-terminal tail of histone protein; the ET domain is not yet fully characterized, and is largely considered to serve a scaffolding function in recruiting diverse transcriptional regulators. Thus, BRD4 plays a key role in regulating gene expression by recruiting relevant transcription modulators to specific genomic loci. Several studies have establish that BRD4 is preferentially located at super-enhancer regions, which often reside upstream of important oncogenes, such as c-MYC, Bcl-xL and BCL-6, and play a key role in regulating their expressions (J. Loven, et al., Cell, 153 (2013) 320-334 and B. Chapuy, et al., Cancer Cell, 24 (2013) 777-790.). Owing to its pivotal role in modulating the expression of essential oncogenes, BRD4 emerges as a promising therapeutic target in multiple cancer types, including midline carcinoma, AML, MM, BL, and prostate cancer (J. Loven, et al., Cell, 153 (2013) 320-334; J. Zuber, et al., Nature, 478 (2011) 524-528; J. E. Delmore, et al., Cell, 146 (2011) 904-917; J. A. Mertz, et al., PNAS, 108 (2011) 16669-16674; A. Wyce, et al., Oncotarget, 4 (2013) 2419-2429; I. A. Asangani, et al., Nature, 510 (2014) 278-282; and C. A. French, et al., Oncogene, 27 (2008) 2237-2242). BRD4's distinct high occupancy of genomic loci proximal to specific oncogenes provide a potential therapeutic window that will allow specific targeting of tumor cells while sparing normal tissues. Particularly, BRD4 may serve as an alternative strategy of targeting c-MYC, which contributes to the development and maintenance of a majority of human cancers but has remained undruggable (J. E. Delmore, et al., Cell, 146 (2011) 904-917; J. A. Mertz, et al., PNAS, 108 (2011) 16669-16674; M. G. Baratta, et al., PNAS, 112(2015)232-237; and M. Gabay, et al., Cold Spring Harb Perspect Med. (2014) 4:a014241).

The development of small molecule BRD4 inhibitors, such as JQ1, iBET and OTX15, has demonstrated promising therapeutic potential in preclinical models of various cancers, including BL (J. Loven, et al., Cell, 153 (2013) 320-334; B. Chapuy, et al., Cancer Cell, 24 (2013) 777-790; J. E. Delmore, et al., Cell, 146 (2011) 904-917; J. A. Mertz, et al., PNAS, 108 (2011) 16669-16674; I. A. Asangani, et al., Nature, 510 (2014) 278-282; M. G. Baratta, et al., PNAS, 112 (2015) 232-237; M. Boi, et al., Clin. Cancer Res., (2015) 21(7):1628-38; and A. Puissant, et al., Cancer discovery, 3 (2013) 308-323). Indeed, BRD4 inhibitors have shown various anti-tumor activities with good tolerability in different mouse tumor models and, not surprisingly, high sensitivity to BRD4 inhibitors such as JQ1, has been associated with high level of either c-MYC and N-MYC in different tumor types, including c-MYC driven BL. Almost all BL cases contain c-myc gene translocation that places it under control of a super-enhancer located upstream of IgH, thus driving an abnormally high level of c-MYC expression, tumor development and maintenance (K. Klapproth, et al., British journal of haematology, 149 (2010) 484-497).

Currently, four BET Bromodomain inhibitors are in phase I clinical trial with focus largely on midline carcinoma and hematological malignancies (CPI-0610, NCT01949883; GSK525762, NCT01587703; OTX015, NCT01713582; TEN-O10, NCT01987362). Preclinical studies with BRD4 inhibitors demonstrate their value in suppressing c-MYC and proliferation in BL cell lines, albeit with IC.sub.50 values often in the range of 100 nM to 1 uM (J. A. Mertz, et al., PNAS, 108 (2011) 16669-16674 and M. Ceribelli, et al., PNAS, 111 (2014) 11365-11370). Thus, despite the rapid progress of BRD4 inhibitors, the effect of BRD4 inhibition has been encouraging, but less than ideal, as the effect is mostly cytostatic and requires relatively high concentration of inhibitors.

An ongoing need exists in the art for effective treatments for disease, especially hyperplasias and cancers, such as multiple myeloma. However, non-specific effects, and the inability to target and modulate certain classes of proteins altogether, such as transcription factors, remain as obstacles to the development of effective anti-cancer agents. As such, small molecule therapeutic agents that leverage or potentiate cereblon's substrate specificity and, at the same time, are “tunable” such that a wide range of protein classes can be targeted and modulated with specificity would be very useful as a therapeutic.

### SUMMARY OF THE DISCLOSURE

Some embodiments relate to a compound having the structure of Formula (I), (IIa) or (IIB).

In one aspect of the invention, disclosed herein are CRBN binding compounds in Tables 1-5, or a pharmaceutically acceptable salt or solvate thereof. In another aspect of the invention, disclosed herein are bifunctional compounds comprising a CRBN binding compound from Tables 1-5.

In another aspect of the invention is a pharmaceutical composition comprising a compound described herein, or a pharmaceutically acceptable salt or solvate thereof, and a pharmaceutically acceptable excipient.

In another aspect of the invention is a pharmaceutical composition comprising a bifunctional compound described herein, or a pharmaceutically acceptable salt or solvate thereof, and a pharmaceutically acceptable excipient.

In another aspect, disclosed herein are methods of inducing a conformational change in CRBN.

In one aspect, the present invention provides a CRBN binding compound that does not cause teratogenicity. In one aspect, the present invention provides a CRBN binding compound that does not degrade SALL4. In one aspect, the present invention provides a CRBN binding compound that does not significantly degrade SALL4.

In another aspect, the present invention provides methods of treating a CRBN-mediated disease or alleviating symptoms in a patient in need thereof.

In another aspect, the invention provides compounds and methods for recruiting a substrate for ubiquitination by an E3 ubiquitination ligase complex comprising CRBN, said method comprising contacting said CRBN with a compound from Tables 1-5.

### DETAILED DESCRIPTION OF THE DISCLOSURE

Ubiquitin-proteasome system is characterized by the E1, E2, and E3 enzyme. First, a ubiquitin molecule is chemically activated in an ATP-dependent manner by an E1-activating enzyme forming a thioester bond between the C-terminal glycine residue of ubiquitin and a conserved cysteine residue of the E1. Then, ubiquitin is transferred on to an E2-conjugated enzyme via a trans-thiolation reaction. Next, an isopeptide bond between the ε-amino group of a substrate lysine residue and the C-terminal glycine residue of ubiquitin is formed via E3 ligase-mediated catalysis and then between ubiquitin molecules to form poly-ubiquitin chains. Upon completion of the ubiquination process, the tagged substrate is subsequently recognized and degraded by the 26S proteasome in an ATP-dependent manner.

In some cases, the E3 ubiquitin ligase family is divided into three families, the HECT (homologous with E6-associated protein C-terminus) family, the RING finger family, and the RBR (RING-between RING_RING) family. HECT E3 enzyme forms a covalent thioester intermediate by accepting a ubiquitin molecule from the E2-ubiquitin via a conserved cysteine residue prior to transferring the ubiquitin molecule to a substrate. RING E3 enzyme directly transfers a ubiquitin molecule to a substrate by bringing both the E2-ubiquitin and the substrate in close proximity to each other. The RBR family recruit E3-ubiquitin conjugated by an N-terminal RING domain and then transfer ubiquitin on to a HECT-type C-terminal catalytic cysteine residue of the E3 before transferring on to the substrate.

In some instances, the RING finger family is further categorized into two subgroups, CRL and APC/C (anaphase-promoting complex/cyclosome). In some cases, the CRL and APC/C subfamilies comprise multi-subunit complexes comprising an adaptor, a substrate receptor subunit, a Cullin scaffold, and a RING-box subunit.

In some embodiments, the CUL4-RBX1-DDB1-CRBN complex (CRL4^{CRBN}) is an E3 ligase that falls under the CRL subgroup of the RING finger family. The CRL4^{CRBN} complex comprises the adaptor protein DDB1, which connects the substrate receptor cereblon (CRBN) to the Cullin 4 (CUL4) scaffold. The Cullin 4 scaffold further binds to RBX1. Upon substrate binding, the CUL4-RBX1-DDB1-CRBN complex bridges the substrate to the E2-ubiquitin to initiate a direct transfer of ubiquitin molecule onto the substrate.

In some instances, thalidomide and related immunomodulatory (IMiD) compounds such as lenalidomide and pomalidomide promote and modulate cereblon recruitment of neosubstrates. For example, a cereblon modulator CC-220 has been shown to improve degradation of Ikaros and Aiolos, two zinc finger transcription factors that have been implicated in lymphoid development and differentiation (Matyskiela, et al., “A cereblon modulator (CC-220) with improved degradation of Ikaros and Aiolos,”J Med Chem. Apr. 20, 2017). Further, dBET1, a bifunctional phthalimide-conjugated ligand which is a substrate for cereblon, selectively targets BRD4, a transcriptional coactivator, for degradation.

In some embodiments, provided herein are substituted isoindolinones. In some aspects, the substituted isoindolinones bind to cereblon. In some aspects, the substituted isoindolinones change the conformation of CRBN in bound cereblon. In some aspects, as cereblon-binding compounds. compound adducts and synthetic ligands that inhibit cereblon-compound adduct formation. In some instances, also provided herein are cereblon binding domains that interact with a compound described herein.

In some embodiments, additionally described herein is a cereblon ligand that modulates recruitment of neosubstrates. In some instances, the method comprises covalent binding of a reactive residue on cereblon for modulation of substrate interaction. In some cases, the method comprises covalent binding of a reactive cysteine residue on cereblon for substrate modulation.

In one aspect of the invention, disclosed herein are CRBN binding compounds in Tables 1-5, or a pharmaceutically acceptable salt or solvate thereof. In another aspect of the invention, disclosed herein are bifunctional compounds comprising a CRBN binding compound from Tables 1-5.

In another aspect of the invention is a pharmaceutical composition comprising a compound described herein, or a pharmaceutically acceptable salt or solvate thereof, and a pharmaceutically acceptable excipient.

In another aspect of the invention is a pharmaceutical composition comprising a bifunctional compound described herein, or a pharmaceutically acceptable salt or solvate thereof, and a pharmaceutically acceptable excipient.

In another aspect, disclosed herein are methods of inducing a conformational change in CRBN.

In one aspect, the present invention provides a CRBN binding compound that does not cause teratogenicity. In one aspect, the present invention provides a CRBN binding compound that does not degrade SALL4. In one aspect, the present invention provides a CRBN binding compound that does not significantly degrade SALL4.

In another aspect, the present invention provides methods of treating a CRBN-mediated disease or alleviating symptoms in a patient in need thereof.

In another aspect, the invention provides compounds and methods for recruiting a substrate for ubiquitination by an E3 ubiquitination ligase complex comprising CRBN, said method comprising contacting said CRBN with a compound from Tables 1-5.

### Compounds

In some embodiments, described herein is a cereblon modulator compound from Tables 1-5, or a pharmaceutically acceptable salt or solvate thereof.

Some embodiments relate to a compound of Formula (I),

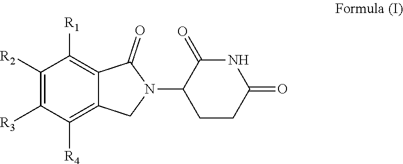

wherein

R¹, R², and R³ are each interpedently selected from hydrogen, halogen, halogenated alkyl, nitro, —COOH, —CONH₂, —CN, —N(R)₂, —OR⁵, —C(═O)R⁵, —C(═O)N(R⁵)₂, —N(R⁵)C(═O)R⁵, —S(═O)₂R⁵, —N(R⁵)S(═O)₂R⁵, —S(═O)₂N(R⁵)₂, —(CH₂)₁₋₆OR⁵, —O(CH₂)₁₋₆-A-R⁵, —O(CH₂)₁₋₆-A-R⁵, —O(CH₂)₁₋₆-A¹-(CH₂)₁₋₆-A²-C(O)OR⁵, substituted or unsubstituted C₁-C₆alkyl, substituted or unsubstituted C₃₋₈ cycloalkyl, substituted or unsubstituted 3-10 membered heterocyclyl, substituted or unsubstituted C₆₋₁₀ aryl, or substituted or unsubstituted 5-10 membered heteroaryl;

R⁴ is selected from hydrogen, halogen, halogenated alkyl, nitro, —COOH, —CONH₂, —CN, —N(C₁₋₆alkyl)₂, —OR⁵, —C(═O)R⁵, —C(═O)N(R⁵)₂, —N(R)C(═O)R⁵, —S(═O)₂R, —N(R)S(═O)₂R³, —S(═O)₂N(R⁵)₂, —(CH₂)₁₋₆OR⁵, —O(CH₂)₁₋₆-A-R⁵, —O(CH₂)₁₋₆-A-R⁵, —O(CH₂)₁₋₆-A¹-(CH₂)₁₋₆-A²-C(O)OR⁵, substituted or unsubstituted C₁-C₆alkyl, substituted or unsubstituted C₃₋₈ cycloalkyl, substituted or unsubstituted 3-10 membered heterocyclyl, substituted or unsubstituted C₆₋₁₀ aryl, or substituted or unsubstituted 5-10 membered heteroaryl;

each A¹ is independently a C₆₋₁₀ arylene or 5-10 membered heteroarylene,

each A² is independently a C₆₋₁₀ arylene, 4-10 membered heterocyclylene, or 4-10 membered heteroarylene, and

each R⁵ is independently H, halogen, substituted or unsubstituted C₁-C₆alkyl, substituted or unsubstituted C₁-C₆haloalkyl, substituted or unsubstituted aryl, or substituted or unsubstituted heteroaryl.

In some embodiments, the compound has the structure of Formula (IIA) or (IIB)

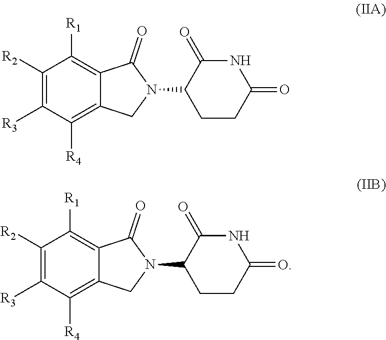

In some embodiments, the compound has the structure of Formula (IIA)

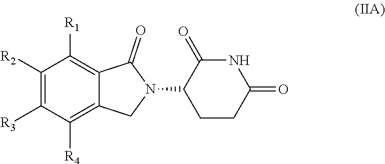

In some embodiments, R1 is H, halogen, unsubstituted C1-C6alkyl; C1-C6alkyl substituted with one or more substituents selected from C6-10 aryl, C3-8 cycloalkyl, 3-10 membered heterocyclyl, 5-10 membered heteroaryl, halogen, and amine: C1-6 alkylene-C6-10 aryl substituted with one or more substituents selected from C1-6 alkyl, alkoxyl, halogen, and amine; and substituted or unsubstituted C1-C6aminoalkyl. In some embodiments, R1 is H, substituted or unsubstituted C1-C6alkyl, or substituted or unsubstituted C1-C6aminoalkyl.

In some embodiments, R2 is H, unsubstituted C1-C6alkyl; C1-C6alkyl substituted with one or more substituents selected from C6-10 aryl, C3-8 cycloalkyl, 3-10 membered heterocyclyl, 5-10 membered heteroaryl, halogen, and amine; C1-6 alkylene-C6-10 aryl substituted with one or more substituents selected from C1-6 alkyl, alkoxyl, halogen, and amine; and substituted or unsubstituted C1-C6aminoalkyl. In some embodiments, R2 is H, substituted or unsubstituted C1-C6alkyl, or substituted or unsubstituted C1-C6aminoalkyl.

In some embodiments, R3 is H, unsubstituted C1-C6alkyl; C1-C6alkyl substituted with one or more substituents selected from C6-10 aryl, C3-8 cycloalkyl, 3-10 membered heterocyclyl, 5-10 membered heteroaryl, halogen, and amine; C1-6 alkylene-C6-10 aryl substituted with one or more substituents selected from C1-6 alkyl, alkoxyl, halogen, and amine; and substituted or unsubstituted C1-C6aminoalkyl. In some embodiments, R3 is H, substituted or unsubstituted C1-C6alkyl, or substituted or unsubstituted C1-C6aminoalkyl.

In some embodiments, R4 is H, unsubstituted C1-C6alkyl; C1-C6alkyl substituted with one or more substituents selected from C6-10 aryl, C3-8 cycloalkyl, 3-10 membered heterocyclyl, 5-10 membered heteroaryl, halogen, and amine; C1-6 alkylene-C6-10 aryl substituted with one or more substituents selected from C1-6 alkyl, alkoxyl, halogen, and amine; and substituted or unsubstituted C1-C6aminoalkyl. In some embodiments, R4 is H, substituted or unsubstituted C1-C6alkyl, or substituted or unsubstituted C1-C6aminoalkyl.

In some embodiments, R5 is H, unsubstituted C1-C6alkyl; C1-C6alkyl substituted with one or more substituents selected from C6-10 aryl, C3-8 cycloalkyl, 3-10 membered heterocyclyl, 5-10 membered heteroaryl, halogen, and amine; C1-6 alkylene-C6-10 aryl substituted with one or more substituents selected from C1-6 alkyl, alkoxyl, halogen, and amine; and substituted or unsubstituted C1-C6aminoalkyl. In some embodiments, R5 is H, substituted or unsubstituted C1-C6alkyl, or substituted or unsubstituted C1-C6aminoalkyl.

In some embodiments, A¹ is phenylene or pyrodylene. In some embodiments, A¹ is a phenylene. In some embodiments, A¹ is a pyrodylene.

In some embodiments, A² is a C₆₋₁₀ arylene. In some embodiments, A² is a 4-10 membered heterocyclylene. In some embodiments, A² is a 4-10 membered heteroarylene. In some embodiments, A² is

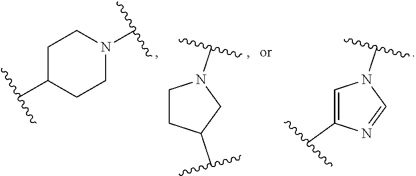 [SMILES: *C1CCN(*)CC1 ; *C1CCN(*)C1 ; *c1cn(*)cn1]

In some embodiments, R¹ hydrogen, halogen, halogenated alkyl, CN, C₁₋₆ alkyl, or alkoxyl.

In some embodiments, R² is halogen, halogenated alkyl, CN, C₁₋₆ alkyl, or alkoxyl.

In some embodiments, R¹, R³, and R⁴ are hydrogen.

In some embodiments, R⁴ is —O(CH₂)₁₋₆-A¹-R⁵, —O(CH₂)₁₋₆-A¹-(CH₂)₁₋₆-A²-C(O)OR⁵.

In some embodiments, R⁵ is H or C₁₋₆ alkyl.

In some embodiments, R¹, R², and R³ are not all hydrogen.

In some embodiments, at least one of R¹, R², and R³ is hydrogen but not all R¹, R², and R³ are hydrogen.

In some embodiments, R² is halogen, halogenated alkyl, or alkoxyl, and R¹, R³, and R⁴ are hydrogen.

In some embodiments, R³ or R⁴ is OCH₂phenyl. In some embodiments, only one of R³ or R⁴ is OCH₂phenyl. In some embodiments, R³ or R⁴ is —NHC(O)C₁₋₆alkyl. In some embodiments, only one of R³ or R⁴ is —NHC(O)C₁₋₆alkyl. In some embodiments, R³ and R⁴ are independently alkoxyl, N(C₁₋₆alkyl)₂, COOH, or CONH₂, SO₂CH₃, phenyl, halogen, or cycloalkyl.

In some embodiments, R¹ is —NHC(O)C₁₋₆alkyl.

In some embodiments, R¹, R², R³, and R⁴ are not all hydrogen. In some embodiments, at least one of R¹, R², R³, and R⁴ is hydrogen.

In some embodiments, R¹ or R⁴ is not NH₂.

In some embodiments, R¹ is H, substituted or unsubstituted C₁-C₆alkyl, or substituted or unsubstituted C₁-C₆aminoalkyl.

In some embodiments, R² is H, unsubstituted C₁-C₆alkyl; C₁-C₆alkyl substituted with one or more substituents selected from C₆₋₁₀ aryl, C₃₋₈ cycloalkyl, 3-10 membered heterocyclyl, 5-10 membered heteroaryl, halogen, and amine; C₁₋₆alkylene-C₆₋₁₀ aryl substituted with one or more substituents selected from C₁₋₆ alkyl, alkoxyl, halogen, and amine; and substituted or unsubstituted C₁-C₆aminoalkyl. In some embodiments, R² is H, substituted or unsubstituted C₁-C₆alkyl, or substituted or unsubstituted C₁-C₆aminoalkyl.

In some embodiments, R³ is H, unsubstituted C₁-C₆alkyl; C₁-C₆alkyl substituted with one or more substituents selected from C₆₋₁₀ aryl, C₃₋₈ cycloalkyl, 3-10 membered heterocyclyl, 5-10 membered heteroaryl, halogen, and amine; C₁₋₆alkylene-C₆₋₁₀ aryl substituted with one or more substituents selected from C₁₋₆ alkyl, alkoxyl, halogen, and amine; and substituted or unsubstituted C₁-C₆aminoalkyl. In some embodiments, R³ is H, substituted or unsubstituted C₁-C₆alkyl, or substituted or unsubstituted C1-C₆aminoalkyl.

In some embodiments, R⁴ is H, unsubstituted C₁-C₆alkyl; C₁-C₆alkyl substituted with one or more substituents selected from C₆₋₁₀ aryl, C₃₋₈ cycloalkyl, 3-10 membered heterocyclyl, 5-10 membered heteroaryl, halogen, and amine; C₁₋₆alkylene-C₆₋₁₀ aryl substituted with one or more substituents selected from C₁₋₆alkyl, alkoxyl, halogen, and amine; and substituted or unsubstituted C₁-C₆aminoalkyl. In some embodiments, R⁴ is H, substituted or unsubstituted C₁-C₆alkyl, or substituted or unsubstituted C₁-C₆aminoalkyl.

In some embodiments, R⁵ is H, unsubstituted C₁-C₆alkyl; C₁-C₆alkyl substituted with one or more substituents selected from C₆₋₄₀ aryl, C₃₋₈ cycloalkyl, 3-10 membered heterocyclyl, 5-10 membered heteroaryl, halogen, and amine; C₁₋₆ alkylene-C6-10 aryl substituted with one or more substituents selected from C₁₋₆ alkyl, alkoxyl, halogen, and amine; and substituted or unsubstituted C₁-C₆aminoalkyl. In some embodiments, R⁵ is H, substituted or unsubstituted C₁-C₆alkyl, or substituted or unsubstituted C₁-C₆aminoalkyl.

In some embodiments, the compound of Formula (I) has a structure selected from Table 1-5.

In some embodiments, the compound of Formula (I) is selected from the group consisting of (R)-3-(6-bromo-1-oxoisoindolin-2-yl)piperidine-2,6-dione, 3-(1-oxo-6-(trifluoromethyl)isoindolin-2-yl)piperidine-2,6-dione, and 3-(6-methoxy-1-oxoisoindolin-2-yl)piperidine-2,6-dione.

In some embodiments, one of (R)-3-(6-bromo-1-oxoisoindolin-2-yl)piperidine-2,6-dione; 3-(1-oxo-6-(trifluoromethyl)isoindolin-2-yl)piperidine-2,6-dione; or 3-(6-methoxy-1-oxoisoindolin-2-yl)piperidine-2,6-dione is used to bind cereblon. In some embodiments, (R)-3-(6-bromo-1-oxoisoindolin-2-yl)piperidine-2,6-dione is used to bind cereblon. In one aspect, one of one of (R)-3-(6-bromo-1-oxoisoindolin-2-yl)piperidine-2,6-dione; 3-(1-oxo-6-(trifluoromethyl)isoindolin-2-yl)piperidine-2,6-dione; or 3-(6-methoxy-1-oxoisoindolin-2-yl)piperidine-2,6-dione degrade neo-substrates.

In one aspect, one of one of (R)-3-(6-bromo-1-oxoisoindolin-2-yl)piperidine-2,6-dione; 3-(1-oxo-6-(trifluoromethyl)isoindolin-2-yl)piperidine-2,6-dione; or 3-(6-methoxy-1-oxoisoindolin-2-yl)piperidine-2,6-dione degrade neo-substrates.

In another aspect, provided herein is a method of inducing a CRBN conformational change or alteration of the properties of a CRBN surface (e.g., on an adjacent region of the protein), comprising contacting the CRBN with a compound from Table 1, wherein said CRBN conformational change or alteration results in a specific biological activity. In one embodiment, the method induces a CRBN conformational change. In a specific embodiment, the CRBN conformational change is within the CMA-binding pocket of the CRBN. In one embodiment, the CRBN conformational change is relative to a CRBN that is bound to a reference compound. In one embodiment, the CRBN that is not bound to a reference compound has a three-dimensional structure as determined by x-ray diffraction having the atomic coordinates set forth in Table 8. In a certain embodiment, the CRBN conformational change is relative to an unbound CRBN. In a specific embodiment, the CRBN conformational change is relative to the CRBN prior to contact with the test compound. In another embodiment, the method induces an alteration of the properties of a CRBN surface. In a specific embodiment, the alteration of the properties of a CRBN surface are on an adjacent region of the protein. In one embodiment, the alteration of the properties of the CRBN surface is relative to a CRBN that is bound to a reference compound. In a certain embodiment, the alteration of the properties of the CRBN surface is relative to an unbound CRBN. In a specific embodiment, the alteration of the properties of the CRBN surface is relative to the CRBN prior to contact with the test compound. In some embodiments, the biological activity is a tumoricidal effect. In other embodiments, the biological activity is an apoptosis effect. In some embodiments, the biological activity is anti-proliferation. In yet other embodiments, the biological activity is PBMC viability. In some embodiments, the biological activity is toxicity. In certain embodiments, the biological activity is substrate degradation. In one embodiments, the biological activity is Aiolos degradation. In another embodiments, the biological activity is Ikaros degradation. In other embodiments, the biological activity is an immune-mediated effect. In another embodiment, the biological activity is IL-2 induction. In some embodiments, the biological activity is IL-2 repression. In yet other embodiments, the biological activity is an effect on fetal hemoglobin (HbF). Any combination of one, two, three or more of the aforementioned biological activities is also contemplated. In certain embodiments, the biological activity is based on specific cell type categories. In other embodiments, the biological activity is based on specific tissue type categories. In yet other embodiments, the biological activity is based on solid tumors or solid tumor categories. In some embodiments, the biological activity is based on non-solid tumor categories. In some embodiments, a CRBN conformational change is induced. In other embodiments, and alteration in the properties of a CRBN surface are induced.

In some embodiments, the biological activity is a tumoricidal effect. In one embodiment, the biological activity is modulation of apoptosis. In other embodiment, the biological activity is modulation of proliferation, e.g., an anti-proliferative effect. In some embodiments, the biological activity is modulation of PBMC viability. In certain embodiments, the biological activity is modulation of toxicity. In other embodiments, the biological activity is substrate degradation. In certain embodiments, the biological activity is degradation of Aiolos and/or Ikaros. In certain embodiments, the biological activity is a prevention of substrate degradation. In some embodiments, the substrate is a CRBN-associated protein. In some embodiments, the biological activity is an immune-mediated effect. In certain embodiments, the biological effect is modulation of IL-2. In yet other embodiments, the biological effect is an effect on fetal hemoglobin (HbF). In some embodiments, the effect is an effect on a CRBN-associated protein.

In certain embodiments, a biological activity is observed in one cell type, but not another cell type. In a specific embodiments, the biological activity is directly correlated with an observed CRBN conformational shift in the cell type(s). In an embodiments, the CRBN conformational shift is in a CMA-binding pocket of CRBN. Such conformational shifts can be assessed using any of the various methods provided elsewhere herein.

In some embodiments, a biological activity is observed in one tissue type, but not another tissue type. In a specific embodiments, the biological activity is directly correlated with an observed CRBN conformational shift in the tissue type(s). In an embodiments, the CRBN conformational shift is in a CMA-binding pocket of CRBN. Such conformational shifts can be assessed using any of the various methods provided elsewhere herein.

In certain embodiments, a biological activity is observed in one tumor (or cancer) type, but not another tumor (or cancer) type. In a specific embodiments, the biological activity is directly correlated with an observed CRBN conformational shift in the tumor (cancer) type(s). In an embodiments, the CRBN conformational shift is in a CMA-binding pocket of CRBN. Such conformational shifts can be assessed using any of the various methods provided elsewhere herein.

In some embodiments, a biological activity is observed in a solid tumor (or cancer), but not in a non-solid tumor (or cancer) (e.g., a hematological tumor). In a specific embodiments, the biological activity is directly correlated with an observed CRBN conformational shift in the tumor(s) (or cancer(s)). In an embodiments, the CRBN conformational shift is in a CMA-binding pocket of CRBN. Such conformational shifts can be assessed using any of the various methods provided elsewhere herein.

In some embodiments, a biological activity is observed in a non-solid tumor (or cancer) (e.g., a hematological tumor), but not in a solid tumor (or cancer). In a specific embodiments, the biological activity is directly correlated with an observed CRBN conformational shift in the tumor(s) (or cancer(s)). In an embodiments, the CRBN conformational shift is in a CMA-binding pocket of CRBN. Such conformational shifts can be assessed using any of the various methods provided elsewhere herein.

In another aspect, provided herein is a method of inducing a CRBN conformational change or alteration of the properties of a CRBN surface (e.g., on an adjacent region of the protein), comprising contacting the CRBN with a compound from Tables 1-5, wherein said CRBN conformational change or alteration results in a specific biological activity.

In another aspect, provided herein is a method of inducing a CRBN conformational change or alteration of the properties of a CRBN surface (e.g., on an adjacent region of the protein), comprising contacting the CRBN with a compound comprising one of (R)-3-(6-bromo-1-oxoisoindolin-2-yl)piperidine-2,6-dione, 3-(1-oxo-6-(trifluoromethyl)isoindolin-2-yl)piperidine-2,6-dione, or 3-(6-methoxy-1-oxoisoindolin-2-yl)piperidine-2,6-dione, wherein said CRBN conformational change or alteration results in a specific biological activity.

In one aspect, compounds and methods of the present invention modulate biomarkers. In some instances, the biomarker is selected from a group consisting of Nestin, KAT1/CCBL1, WIBG, MVP, PARP4, ZFP91, and ZNF198. In some embodiments of the various methods provided herein, the biomarker is selected from a group consisting of Nestin, KAT1/CCBL 1, and WIBG. In other embodiments, the biomarker is selected from a group consisting of MVP, PARP4, ZFP91, and ZNF198.

In some embodiments of the various methods provided herein, the biomarker is AHNAK, ALOX5, AMPD3, ANXA4, ANXA6, ARHGAP19, ASNS, ASPM, ATP2B4, B4GALT3, BANK1, BCDIN3D, BLZF1, BMF, BST2, C10orf76, C19orf66, CA2, CA8, CAMSAP3, CCDC69, CCNB1, CD36, CDC7, CDCA3, CENPF, CLN3, CNN3, CORO1B, CPNE2, CRBN, CSNK1A1, CSRP2, CTNND1, CTSH, DAPK2, DDX58, DHPS, DHX58, DLG2, DLGAP5, DOK3, DTX3L, ECT2, EFCAB4B, EHMT1, EHMT2, EIF2AK2, EPB41L1, EPCAM, ESRP1, ETV6, EXTL2, F13A1, FAM195A, FAM65B, FBRSL1, FCGR2B, FES, FHOD1, FIGNL1, FMNL3, GBP1, GMFG, GMPR, GPT2, GRAMD1A, GRAMD1B, GRPEL2, HIP1, HJURP, HLA-B, HLA-DMA, HMCES, HMMR, HOXC4, HPSE, ICAM2, ID3, IF135, IFIH1, IFIT1, IFIT3, IFIT5, IFITM2, IKZF1, IKZF3, IL4I1, IRF7, IRF9, IRS2, ISG15, ISG20, ITGB7, JAK3, KIF18B, KIF22, KIF2C, LAP3, LGALS1, LGALS3BP, LIMD1, LIPG, LPXN, MAN2A2, MARCKS, MFI2, MGARP, MINA, MIS18BP1, MOV10, MPP7, MUC1, MX1, MX2, MYO1G, NCF2, NEIL1, NFKBID, NME3, NMI, NPIPB5, NT5C3A, OAS1, OAS2, OAS3, OMA1, ORC6, PARP14, PARP9, PARVB, PBK, PBXIP1, PDE6D, PKMYT1, PLD4, PLEKHO1, PLK1, PLSCR1, PLXNB2, PODXL, PODXL2, POLE2, POMP, PPFIBP1, PRDM15, PRNP, PTAFR, PTMS, PTTG1, PYROXD1, QPRT, RAB13, RASA4B, RASSF6, RCN1, RGCC, RGS1, RGS2, RNF213, S100A13, SAMD9L, SAMIHD1, SEC14L1, SERPINH1, SGOL1, SGOL2, SLCO3A1, SLCO4A1, SLFN11, SLFN13, SLFN5, SP110, SP140, SPN, SPR, STAP1, STAT1, STAT2, TACC3, TAP1, TAX1BP3, THEMIS2, THTPA, TIMM8B, TNFAIP8L2, TNFSF8, TOP2A, TP5313, TPX2, TREX1, TRIB3, TRIM22, TTC39C, TXNIP, UBA7, UBE2L6, USP41, VCL, VNN2, WIZ, WSB1, WWC1, ZBTB38, ZFP91, ZMYM2, ZNF385B, ZNF581 or ZNF644. In certain embodiments, any combination of two or more of the above-identified biomarkers is also contemplated.

In other embodiments of the various methods provided herein, the biomarker is selected from a group consisting of AHNAK, ALOX5, AMPD3, ANXA4, ANXA6, ATP2B4, BMF, BST2, C10orf76, C19orf66, CD36, CLN3, CNN3, CORO1B, CPNE2, CRBN, CSRP2, CTNND1, CTSH, DAPK2, DDX58, DHX58, DLG2, DTX3L, EIF2AK2, EPB4IL1, ETV6, EXTL2, F13A 1, FAM65B, FCGR2B, FES, FMNL3, GBP1, GMFG, GMPR, HIP1, HLA-B, HLA-DMA, HPSE, ID3, IFI35, IFIH1, IFIT1, IFIT3, IFIT5, IFITM2, IL4I1, IRF7, IRF9, ISG15, ISG20, ITGB7, JAK3, LAP3, LGALS1, LGALS3BP, LIMD1, MAN2A2, MARCKS, MF12, MGARP, MOV10, MPP7, MUC1, MX1, MX2, MYO1G, NCF2, NME3, NMI, NT5C3A, OAS1, OAS2, OAS3, PARP14, PARP9, PBXIP1, PLD4, PLEKHO1, PLSCR1, PLXNB2, POMP, PPFIBP1, PTMS, QPRT, RAB13, RCN1, RGCC, RNF213, S100A13, SAMD9L, SAMHD1, SERPINH1, SLFN11, SLFN13, SLFN5, SP110, SP140, SPN, SPR, STAP1, STAT1, STAT2, TAP1, TAX1BP3, THEMIS2, THTPA, TNFAIP8L2, TNFSF8, TP53I3, TREX1, TRIM22, TTC39C, TXNIP, UBA7, UBE2L6, USP41, VCL, VNN2 and ZBTB38.

In yet other embodiments of the various methods provided herein, the biomarker is selected from a group consisting of ARHGAP19, ASNS, ASPM, B4GALT3, BANK1, BCDIN3D, BLZF1, CA2, CA8, CAMSAP3, CCDC69, CCNB1, CDC7, CDCA3, CENPF, CSNK1A1DHPS, DLGAP5, DOK3, ECT2, EFCAB4B, EHMT1, EHMT2, EPCAM, ESRP1, FAM195A, FBRSL1, FHOD1, FIGNL1, GPT2, GRAMD1A, GRAMD1B, GRPEL2, HJURP, HMCES, HMMR, HOXC4, ICAM2, IKZF1, IKZF3, IRS2, KIF18B, KIF22, KIF2C, LIPG, LPXN, MINA, MIS18BP1, NEIL1, NFKBID, NPIPB5, OMA1, ORC6, PARVB, PBK, PDE6D, PKMYT1, PLK1, PODXL, PODXL2, POLE2, PRDM15, PRNP, PTAFR, PTTG1, PYROXD1, RASA4B, RASSF6, RGS1, RGS2, SEC14L1, SGOL1, SGOL2, SLCO3A1, SLCO4A1, TACC3, TIMM8B, TOP2A, TPX2, TRIB3, WIZ, WSB1, WWC1, ZFP91, ZMYM2, ZNF385B, ZNF581 and ZNF644.

In yet other embodiments of the various methods provided herein, the biomarker is selected from a group consisting of SEPT2, A2M, A2mp, ACSM2B, ADAM22, AFP, AGMAT, AGT, Ahsg, AHSG, ALB, Amacr, AMACR, ANPEP, Anxa5, ANXA9, APBB2, APCS, AQP1, ATHL1, Atp5c1, C11orf52, C3, C4A, CASR, CCBL1, CCDC28A, Cct3, CD74, CDH16, CEACAM1, CLN3, COX5A, CPVL, CRBN, CRYM, CTSL, CTUI, DAPK2, DLG5, DUSP23, ELF3, Eps15, ERBB3, FAM83H, Fkbp9, FLRT3, FUK, GC, GPD1, GPR39, HMBOX1, HYPK, ITFG2, Itgb1, ITIH1, ITIH4, KANSL3, KCTD14, KLC4, KPTN, Lamp2, LGALS2, LGALS9, LIG3, LMBRD1, LOC100996516, LPL, LYN, MAP2K7, MLLT6, Mogs, Mrps22, MYRF, NCOA3, NR2C1, PAH, PDZK1, Peg10, PLEKHO2, Postn, POSTN, PPP1R13L, Prdx3, PRODH2, Ptbp3, RBKS, RGS14, RHBDD2, RNF126, RNF7, Rp19, Rp1p1, Rp1p2, RSBN1L, SAT2, SEPHS1, SERF2, Serpina10, Serpinf1, SLC25A51, SLC29A3, SPAG7, Suclg2, SULT1A1, SULT1A3, To11ip, Top2a, Trap1, TSPAN31, TTC13, Upp1, USP30, Vcam1, VIL1, Vim, and ZNF770. In certain embodiments, any combination of two or more of the above-identified biomarkers is also contemplated.

In yet other embodiments of the various methods provided herein, the biomarker is selected from a group consisting of MARCH7, SEPT2, Abcc1, ABCG1, ABCG2, Adam10, AFF3, Akap9, AP1AR, APOL1, ATP11A, BANP, BLZF1, C10orf118, C4b, C6orf57, CAST, CCAR2, CCBL1, CCDCl38, CCDC71L, CD33, CD40, CEACAM1, CHKB, CHURC1, CLK4, Col15a1, CPN1, CSRP2BP, CXorf67, DGAT1, DHRS2, DLG5, Eif2s1, ENTPD5, EPDR1, Eps812, Epx, FAM111A, FAM120B, FAM206A, FAM83H, Fasn, FASTKD3, FCGRT, FLII, FNBPaL, Gak, Gatad2a, Gm906, GPBP1L1, Gtpbp4, HABP4, HIRIP3, HK1, HMGXB3, HSP90AA4P, IGDCC4, IL36B, IMMT, INA, KCTD18, KIDINS220, KIF16B, KLC4, KLHL24, LAMTOR5, LARP1B, LGALS9, LMBRD1, Lta4h, LYRM1, MAT1A, MBIP, MBP, METTL21A, MLLT1, MPV17L2, MTIF3, MTRF1L, MYO5B, N4BP2, NABP2, NAP1L1, Ndufa10, Ndufa2, Ndufs2, NFRKB, NSMCE2, NTHL1, Nup37, OVOS1, PDCD2, Pgd, PLA2G4A, PLEC, PLXNA3, POLG, POLK, PORCN, PPAPDC2, Ppp2r4, RBPMS2, RNF4, SAMD1, SATB2, SELK, SERBP1, Serpina10, Serpinc1, Serpinf2, Serping1, SGCB, SIRT7, SLC50A1, SMCR8, SPAG1, SPECC1, SUV39H1, Sypl1, TAPBPL, Tars, Tars12, TCF3, TEC, TERF1, TEX9, TGFBRAP1, TMC6, TMEM120A, TMEM179B, TMEM50A, TOPORS, TOR4A, TRAPPC9, Tspan9, TSPYL1, TUBB4A, TXNDC11, UGCG, Vps37c, VPS54, VRK3, XRRA1, YPEL5, ZBTB1, ZBTB40, ZDHHC3, ZHX1, ZNF292, and ZNF830. In certain embodiments, any combination of two or more of the above-identified biomarkers is also contemplated.

In yet other embodiments of the various methods provided herein, the biomarker is selected from a group consisting of ABHD6, ACVR1, AGR2, AHNAK2, AKAP12, ANLN, AP5S1, ARL4C, ARL6IP1, ARPIN, ASH1L, AXL, C4orf3, CANX, CD44, CD46, CD59, CDC45, CENPK, CEP55, COL6A3, CPA4, CTNNAL1, CYP27A1, CYR61, DEGS1, DHX40, EHD2, EPHA2, EREG, ETHE1, FAM160A1, FAM172A, FOSL1, GHDC, GJA1, GLRX, GLS, GNG2, GNG5, GRB10, GRPEL2, Tap, Igf2r, IGFBP1, IGFBP3, IKBKE, JAG1, KIAA0100, KIAA1462, KIF20B, KIF22, KLC2, KRT9, LDLR, LPXN, MAFF, MELK, MET, MGAT4B, MGLL, MMP7, MST1R, MT2A, MVP, MYOF, MYOF, NDRG1, NNMT, NTN4, OTUB2, PALMD, PANK1, PARP4, PBK, PDE6D, PHC3, PHLDA1, PLA2G4A, PLCD4, PPIL4, PRC1, Prkca, PRKCDBP, PTGES2, PTGS1, RUNX2, SCD, SDSL, SEL1L3, SEMA3A, SERPINE1, SIRPA, SKA1, SLC14A1, SLC25A29, SLC2A1, SLC34A2, SLC34A2, SLC38A2, SLC4A7, SMAD3, SPANXA1, SQSTM1, TIMM8A, TK1, TMEM56, TMSB10, TNFRSF12A, TRIM44, TRIM65, TSC22D1, UBE2C, UBE2E2, UPK1B, VASP, VPS13B, WIZ, YBX3, ZFP91, and ZMYM2. In certain embodiments, any combination of two or more of the above-identified biomarkers is also contemplated.

In yet other embodiments of the various methods provided herein, the biomarker is selected from a group consisting of ACSL6, AHSG, ALB, ARPP19, ATP5EP2, ATP5I, ATP5J, AZGP1, BCKDK, BLOCIS3, BTF3, C2orf76, CABLES2, CAPN15, CCDC88C, CDH1, CETN2, CLTC, COGS, COX17, COX7B, Cpsf1, CSRP2, CXXC1, CYC1, DAP3, DNAJC19, DNM2, DPY30, EHMT2, FAM162A, FAM84B, FAM98A, FER, FKBP2, FUNDC2, GK5, GLRX, Gm14139, GOLT1B, GPATCH3, Hbb-b2, HIST1h1C, Hist1h1e, HIST1H1E, HIST1H2AH, HIST1H2BC, HMGB2, HMGN3, HSP90B1, HSPB11, IKBKE, JAGN1, Kxd1, LAMA1, LPXN, MAFG, MAGEA10, MED11, MED18, METTL5, MFSD1, MGAT4B, MGST1, MGST2, MRPL9, MRPS18C, MRPS21, Mt2, MT2A, MT-ATP8, MVP, MYO1F, NDUFB1, NME7, NMES1, NOMO2, NT5C2, Nt5dc2, NT5E, NUDT3, NUDT5, OASL, PARP4, Parp4, PCK1, PDE6D, PFN2, PGPEP1, PIGK, PLOD1, Prdx2, PTBP2, PTDSS1, RAB28, RAB4B, RAB8B, RNF166, ROBO1, RPL13A, Rp118, RPL18A, RPL19, RPL22, RPL28, RPL35, RPL36AL, RPL37, Rp17, RPL8, RPS13, RPS15, RPS17L, RPS19, RPS25, RPS29, RTN4IP1, S100A11, SDHAF2, SDSL, Sec13, SERF2, SIK2, SLC20A2, SLC25A3, SLC25A6, SLC52A2, SNRPF, SPANXA1, SPPL2B, STIM2, STK11, SYTL4, TAF15, TBCID12, TFAP4, TMBIM6, TMCO1, TMED9, TMEM189, TMEM222, TPM1, TWSG1, UBE3B, VAPA, VHL, VNN1, ZDHHC20, ZEB1, ZFP91, and ZMYM2. In certain embodiments, any combination of two or more of the above-identified biomarkers is also contemplated.

TABLE 1

| Chemical Structure | Name |
|---|---|
| 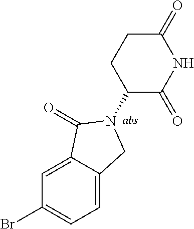 | (R)-3-(6-bromo-1- oxoisoindolin-2-yl)piperidine- 2,6-dione |
| 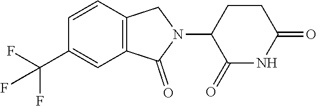 | 3-(1-oxo-6- (trifluroomethyl)isoindolin-2- yl)piperidine-2,6-dione |
| 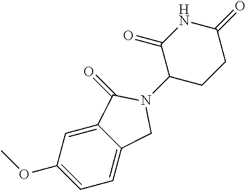 [SMILES: COc1ccc2c(c1)C(=O)N(C1CCC(=O)NC1=O)C2] | 3-(6-methoxy-1- oxoisoindolin-2-yl)piperidine- 2,6-dione |
| 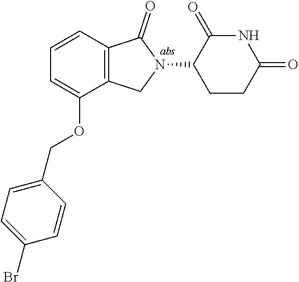 | (S)-3-(4-((4- bromobenzyl)oxy)-1- oxoisoindolin-2-yl)piperidine- 2,6-dione |
| 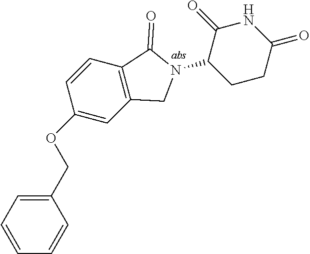 | (S)-3-(5-(benzyloxy)-1- oxoisoindolin-2-yl)piperidine- 2,6-dione |
| 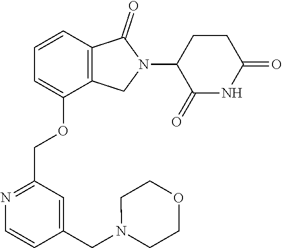 [SMILES: O=C1CCC(N2Cc3c(OCc4cc(CN5CCOCC5)ccn4)cccc3C2=O)C(=O)N1] | 3-(4-((4- (morpholinomethyl)pyridin-2- yl)methoxy)-1-oxoisoindolin- 2-yl)piperidine-2,6-dione |
| 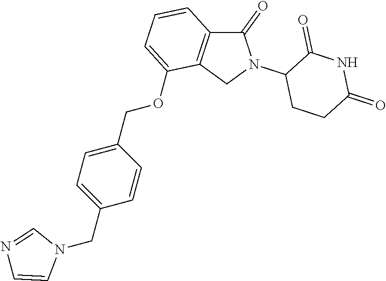 [SMILES: O=C1CCC(N2Cc3c(OCc4ccc(Cn5ccnc5)cc4)cccc3C2=O)C(=O)N1] | 3-(4-((4-((1H-imidazol-1- yl)methyl)benzyl)oxy)-1- oxoisoindolin-2-yl)piperidine- 2,6-dione |
| 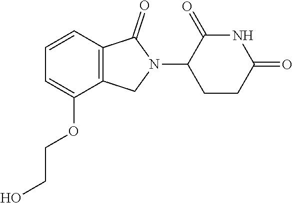 [SMILES: O=C1CCC(N2Cc3c(OCCO)cccc3C2=O)C(=O)N1] | 3-(4-(2-hydroxyethoxy)-1- oxoisoindolin-2-yl)piperidine- 2,6-dione |
| 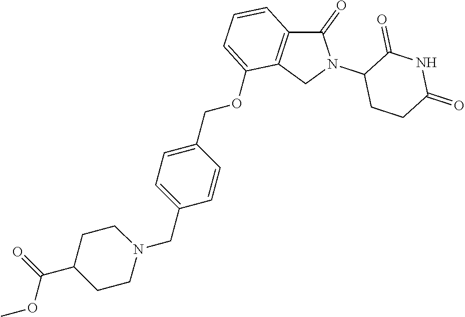 [SMILES: COC(=O)C1CCN(Cc2ccc(COc3cccc4c3CN(C3CCC(=O)NC3=O)C4=O)cc2)CC1] | methyl 1-(4-(((2-(2,6- dioxopiperidin-3-yl)-1- oxoisoindolin-4- yl)oxy)methyl)benzyl)piperidine- 4-carboxylate |
| 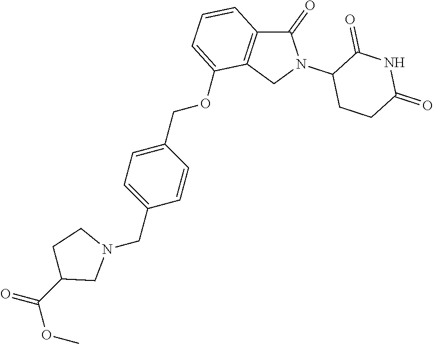 [SMILES: COC(=O)C1CCN(Cc2ccc(COc3cccc4c3CN(C3CCC(=O)NC3=O)C4=O)cc2)C1] | methyl 1-(4-(((2-(2,6- dioxopiperidin-3-yl)-1- oxoisoindolin-4- yl)oxy)methyl)benzyl)pyrrolidin- 3-carboxylate |
| 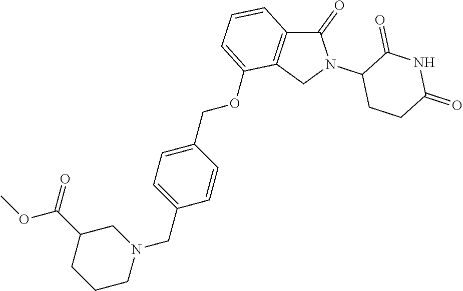 [SMILES: COC(=O)C1CCCN(Cc2ccc(COc3cccc4c3CN(C3CCC(=O)NC3=O)C4=O)cc2)C1] | methyl 1-(4-(((2-(2,6- dioxopiperidin-3-yl)-1- oxoisoindolin-4- yl)oxy)methyl)benzyl)piperidine- 3-carboxylate |
| 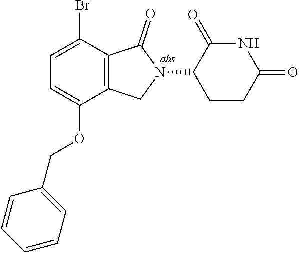 | (S)-3-(4-(benzyloxy)-7- bromo-1-oxoisoindolin-2- yl)piperidine-2,6-dione |
| 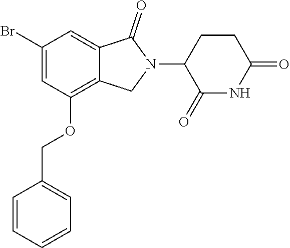 [SMILES: O=C1CCC(N2Cc3c(OCc4ccccc4)cc(Br)cc3C2=O)C(=O)N1] | 3-(4-(benzyloxy)-6-bromo-1- oxoisoindolin-2-yl)piperidine- 2,6-dione |
| 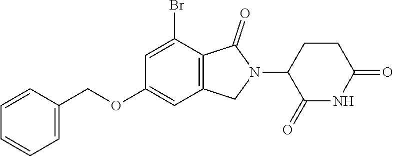 [SMILES: O=C1CCC(N2Cc3cc(OCc4ccccc4)cc(Br)c3C2=O)C(=O)N1] | 3-(5-(benzyloxy)-7-bromo-1- oxoisoindolin-2-yl)piperidine- 2,6-dione |
| 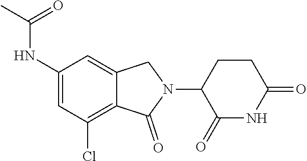 [SMILES: CC(=O)Nc1cc(Cl)c2c(c1)CN(C1CCC(=O)NC1=O)C2=O] | N-(7-chloro-2-(2,6- dioxopiperidin-3-yl)-1- oxoisoindolin-5-yl)acetamide |
| 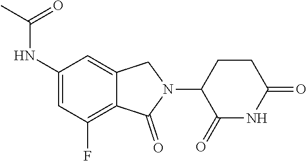 [SMILES: CC(=O)Nc1cc(F)c2c(c1)CN(C1CCC(=O)NC1=O)C2=O] | N-(2-(2,6-dioxopiperidin-3- yl)-7-fluoro-1-oxoisoindolin- 5-yl)acetamide |
| 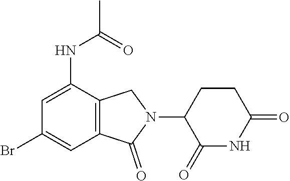 [SMILES: CC(=O)Nc1cc(Br)cc2c1CN(C1CCC(=O)NC1=O)C2=O] | N-(6-bromo-2-(2,6- dioxopiperidin-3-yl)-1- oxoisoindolin-4-yl)acetamide |
| 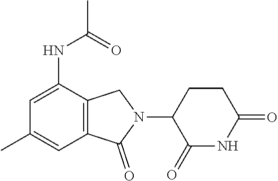 [SMILES: CC(=O)Nc1cc(C)cc2c1CN(C1CCC(=O)NC1=O)C2=O] | N-(2-(2,6-dioxopiperidin-3- yl)-6-methyl-1-oxoisoindolin- 4-yl)acetamide |
| 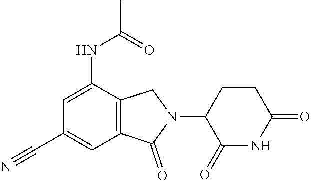 [SMILES: CC(=O)Nc1cc(C#N)cc2c1CN(C1CCC(=O)NC1=O)C2=O] | N-(6-cyano-2-(2,6- dioxopiperidin-3-yl)-1- oxoisoindolin-4-yl)acetamide |
| 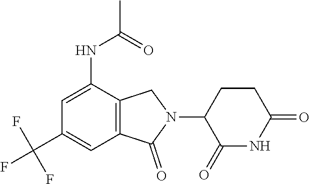 [SMILES: CC(=O)Nc1cc(C(F)(F)F)cc2c1CN(C1CCC(=O)NC1=O)C2=O] | N-(2-(2,6-dioxopiperidin-3- yl)-1-oxo-6- (trifluoromethyl)isoindolin-4- yl)acetamide |
| 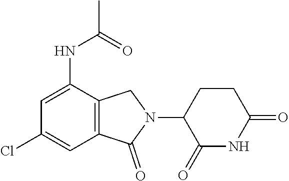 [SMILES: CC(=O)Nc1cc(Cl)cc2c1CN(C1CCC(=O)NC1=O)C2=O] | N-(6-chloro-2-(2,6- dioxopiperidin-3-yl)-1- oxoisoindolin-4-yl)acetamide |
| 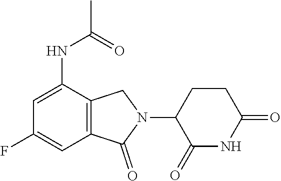 [SMILES: CC(=O)Nc1cc(F)cc2c1CN(C1CCC(=O)NC1=O)C2=O] | N-(2-(2,6-dioxopiperidin-3- yl)-6-fluoro-1-oxoisoindolin- 4-yl)acetamide |
| 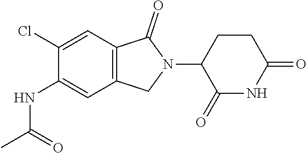 [SMILES: CC(=O)Nc1cc2c(cc1Cl)C(=O)N(C1CCC(=O)NC1=O)C2] | N-(6-chloro-2-(2,6- dioxopiperidin-3-yl)-1- oxoisoindolin-5-yl)acetamide |
| 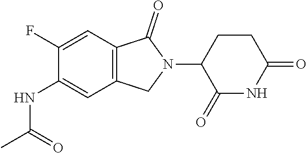 [SMILES: CC(=O)Nc1cc2c(cc1F)C(=O)N(C1CCC(=O)NC1=O)C2] | N-(2-(2,6-dioxopiperidin-3- yl)-6-fluoro-1-oxoisoindolin- 5-yl)acetamide |
| 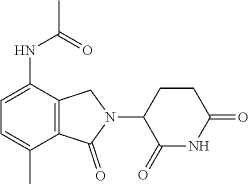 [SMILES: CC(=O)Nc1ccc(C)c2c1CN(C1CCC(=O)NC1=O)C2=O] | N-(2-(2,6-dioxopiperidin-3- yl)-7-methyl-1-oxoisoindolin- 4-yl)acetamide |
| 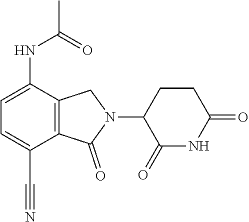 [SMILES: CC(=O)Nc1ccc(C#N)c2c1CN(C1CCC(=O)NC1=O)C2=O] | N-(7-cyano-2-(2,6- doxopiperidin-3-yl)-1- oxoisoindolin-4-yl)acetamide |
| 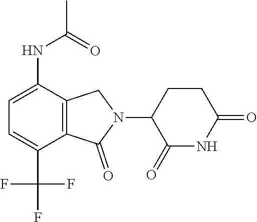 [SMILES: CC(=O)Nc1ccc(C(F)(F)F)c2c1CN(C1CCC(=O)NC1=O)C2=O] | N-(2-(2,6-dioxopiperidin-3- yl)-1-oxo-7- (trifluoromethyl)isoindolin-4- yl)acetamide |
| 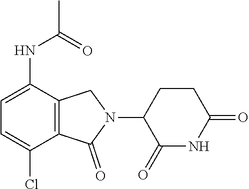 [SMILES: CC(=O)Nc1ccc(Cl)c2c1CN(C1CCC(=O)NC1=O)C2=O] | N-(7-chloro-2-(2,6- dioxopiperidin-3-yl)-1- oxoisoindolin-4-yl)acetamide |
| 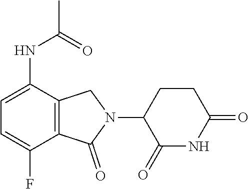 [SMILES: CC(=O)Nc1ccc(F)c2c1CN(C1CCC(=O)NC1=O)C2=O] | N-(2-(2,6-dioxopiperidin-3- yl)-7-fluoro-1-oxoisoindolin- 4-yl)acetamide |
| 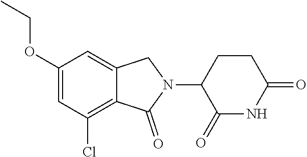 [SMILES: CCOc1cc(Cl)c2c(c1)CN(C1CCC(=O)NC1=O)C2=O] | 3-(7-chloro-5-ethoxy-1- oxoisoindolin-2-yl)piperidine- 2,6-dione |
| 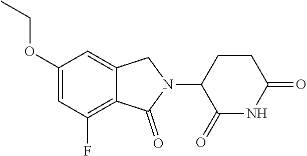 [SMILES: CCOc1cc(F)c2c(c1)CN(C1CCC(=O)NC1=O)C2=O] | 3-(5-ethoxy-7-fluoro-1- oxoisoindolin-2-yl)piperidine- 2,6-dione |
| 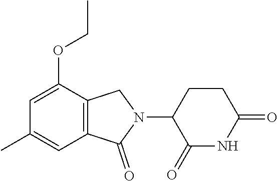 [SMILES: CCOc1cc(C)cc2c1CN(C1CCC(=O)NC1=O)C2=O] | 3-(4-ethoxy-6-methyl-1- oxoisoindolin-2-yl)piperidine- 2,6-dione |
| 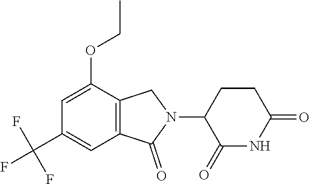 [SMILES: CCOc1cc(C(F)(F)F)cc2c1CN(C1CCC(=O)NC1=O)C2=O] | 3-(4-ethoxy-1-oxo-6- (trifluoromethyl)isoindolin-2- yl)piperidine-2,6-dione |
| 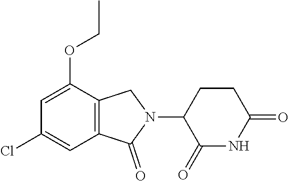 [SMILES: CCOc1cc(Cl)cc2c1CN(C1CCC(=O)NC1=O)C2=O] | 3-(6-chloro-4-ethoxy-1- oxoisoindolin-2-yl)piperidine- 2,6-dione |
| 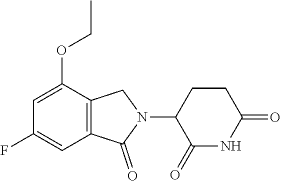 [SMILES: CCOc1cc(F)cc2c1CN(C1CCC(=O)NC1=O)C2=O] | 3-(4-ethoxy-6-fluoro-1- oxoisoindolin-2-yl)piperidine- 2,6-dione |
| 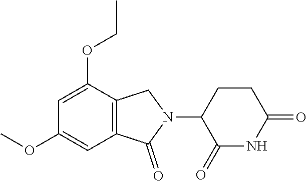 [SMILES: CCOc1cc(OC)cc2c1CN(C1CCC(=O)NC1=O)C2=O] | 3-(4-ethoxy-6-methoxy-1- oxoisoindolin-2-yl)piperidine- 2,6-dione |
| 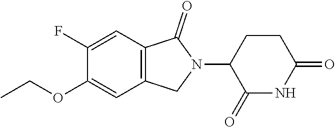 [SMILES: CCOc1cc2c(cc1F)C(=O)N(C1CCC(=O)NC1=O)C2] | 3-(5-ethoxy-6-fluoro-1- oxoisoindolin-2-yl)piperidine- 2,6-dione |
| 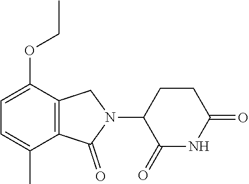 [SMILES: CCOc1ccc(C)c2c1CN(C1CCC(=O)NC1=O)C2=O] | 3-(4-ethoxy-7-methyl-1- oxoisoindolin-2-yl)piperidine- 2,6-dione |
| 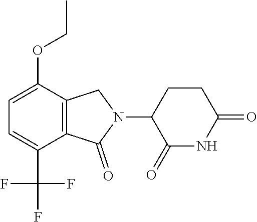 [SMILES: CCOc1ccc(C(F)(F)F)c2c1CN(C1CCC(=O)NC1=O)C2=O] | 3-(4-ethoxy-1-oxo-7- (trifluoromethyl)isoindolin-2- yl)piperidine-2,6-dione |
| 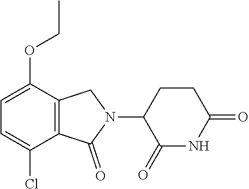 [SMILES: CCOc1ccc(Cl)c2c1CN(C1CCC(=O)NC1=O)C2=O] | 3-(7-chloro-4-ethoxy-1- oxoisoindolin-2-yl)piperidine- 2,6-dione |
| 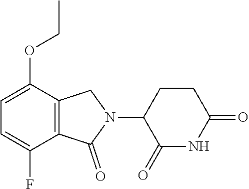 [SMILES: CCOc1ccc(F)c2c1CN(C1CCC(=O)NC1=O)C2=O] | 3-(4-ethoxy-7-fluoro-1- oxoisoindolin-2-yl)piperidine- 2,6-dione |
| 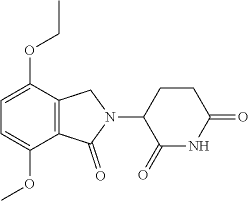 [SMILES: CCOc1ccc(OC)c2c1CN(C1CCC(=O)NC1=O)C2=O] | 3-(4-ethoxy-7-methoxy-1- oxoisoindolin-2-yl)piperidine- 2,6-dione |
| 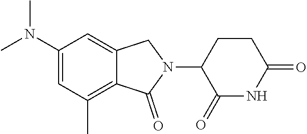 [SMILES: Cc1cc(N(C)C)cc2c1C(=O)N(C1CCC(=O)NC1=O)C2] | 3-(5-(dimethylamino)-7- methyl-1-oxoisoindolin-2- yl)piperidine-2,6-dione |
| 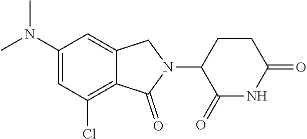 [SMILES: CN(C)c1cc(Cl)c2c(c1)CN(C1CCC(=O)NC1=O)C2=O] | 3-(7-chloro-5- (dimethylamino)-1- oxoisoindolin-2-yl)piperidine- 2,6-dione |
| 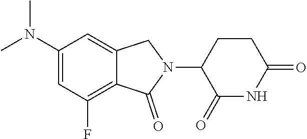 [SMILES: CN(C)c1cc(F)c2c(c1)CN(C1CCC(=O)NC1=O)C2=O] | 3-(5-(dimethylamino)-7- fluoro-1-oxoisoindolin-2- yl)piperidine-2,6-dione |
| 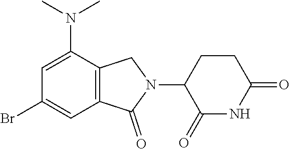 [SMILES: CN(C)c1cc(Br)cc2c1CN(C1CCC(=O)NC1=O)C2=O] | 3-(6-bromo-4- (dimethylamino)-1- oxoisoindolin-2-yl)piperidine- 2,6-dione |
| 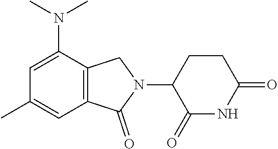 [SMILES: Cc1cc2c(c(N(C)C)c1)CN(C1CCC(=O)NC1=O)C2=O] | 3-(4-(dimethylamino)-6- methyl-1-oxoisoindolin-2- yl)piperidine-2,6-dione |
| 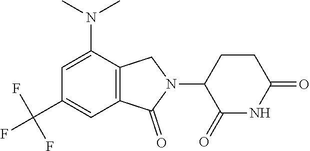 [SMILES: CN(C)c1cc(C(F)(F)F)cc2c1CN(C1CCC(=O)NC1=O)C2=O] | 3-(4-(dimethyalmino)-1-oxo- 6-(trifluoromethyl)isoindolin- 2-yl)piperidine-2,6-dione |
| 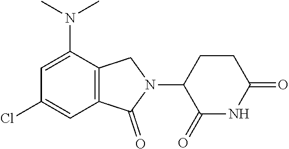 [SMILES: CN(C)c1cc(Cl)cc2c1CN(C1CCC(=O)NC1=O)C2=O] | 3-(6-chloro-4- (dimethylamino)-1- oxoisoindolin-2-yl)piperidine- 2,6-dione |
| 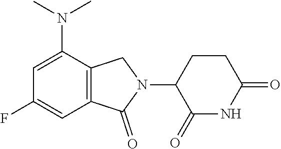 [SMILES: CN(C)c1cc(F)cc2c1CN(C1CCC(=O)NC1=O)C2=O] | 3-(4-(dimethylamino)-6- fluoro-1-oxoisoindolin-2- yl)piperidine-2,6-dione |
| 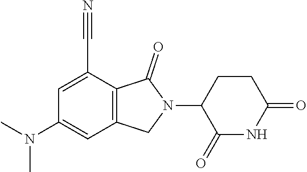 [SMILES: CN(C)c1cc(C#N)c2c(c1)CN(C1CCC(=O)NC1=O)C2=O] | 6-(dimethylamino)-2-(2,6- dioxopiperidin-3-yl)-3- oxoisoindoline-4-carbonitrile |
| 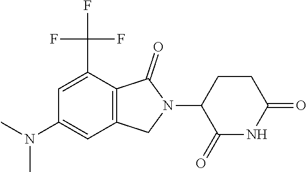 [SMILES: CN(C)c1cc2c(c(C(F)(F)F)c1)C(=O)N(C1CCC(=O)NC1=O)C2] | 3-(5-(dimethylamino)-1-oxo- 7-(trifluoromethyl)isoindolin- 2-yl)piperidine-2,6-dione |
| 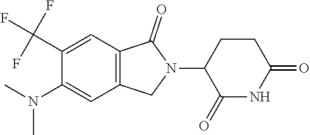 [SMILES: CN(C)c1cc2c(cc1C(F)(F)F)C(=O)N(C1CCC(=O)NC1=O)C2] | 3-(5-(dimethylamino)-1-oxo- 6-(trifluoromethyl)isoindolin- 2-yl)piperidine-2,6-dione |
| 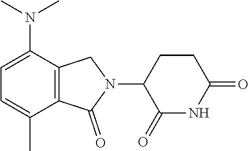 [SMILES: Cc1ccc(N(C)C)c2c1C(=O)N(C1CCC(=O)NC1=O)C2] | 3-(4-(dimethylamino)-7- methyl-1-oxoisoindolin-2- yl)piperidine-2,6-dione |
| 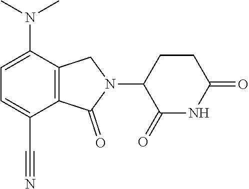 [SMILES: CN(C)c1ccc(C#N)c2c1CN(C1CCC(=O)NC1=O)C2=O] | 7-(dimethylamino)-2-(2,6- dioxopiperidin-3-yl)-3- oxoisoindoline-4-carbonitrile |
| 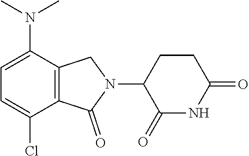 [SMILES: CN(C)c1ccc(Cl)c2c1CN(C1CCC(=O)NC1=O)C2=O] | 3-(7-chloro-4- (dimethyalmino)-1- oxoisoindolin-2-yl)piperidine- 2,6-dione |
| 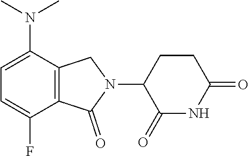 [SMILES: CN(C)c1ccc(F)c2c1CN(C1CCC(=O)NC1=O)C2=O] | 3-(4-(dimethyalmino)-7- fluoro-1-oxoisoindolin-2- yl)piperidine-2,6-dione |
| 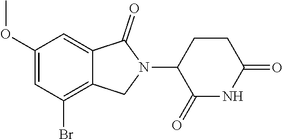 [SMILES: COc1cc(Br)c2c(c1)C(=O)N(C1CCC(=O)NC1=O)C2] | 3-(4-bromo-6-methoxy-1- oxoisoindolin-2-yl)piperidine- 2,6-dione |
| 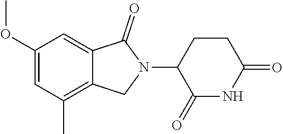 [SMILES: COc1cc(C)c2c(c1)C(=O)N(C1CCC(=O)NC1=O)C2] | 3-(6-methxoy-4-methyl-1- oxoisoindolin-2-yl)piperidine- 2,6-dione |
| 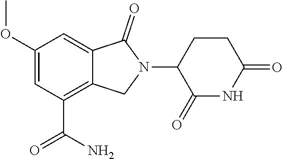 [SMILES: COc1cc(C(N)=O)c2c(c1)C(=O)N(C1CCC(=O)NC1=O)C2] | 2-(2,6-dioxopiperidin-3-yl)-6- methoxy-1-oxoisoindoline-4- carboxamide |
| 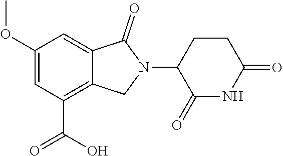 [SMILES: COc1cc(C(=O)O)c2c(c1)C(=O)N(C1CCC(=O)NC1=O)C2] | 2-(2,6-dioxopiperidin-3-yl)-6- methoxy-1-oxoisoindoline-4- carboxylic acid |
| 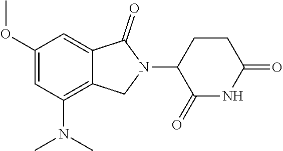 [SMILES: COc1cc2c(c(N(C)C)c1)CN(C1CCC(=O)NC1=O)C2=O] | 3-(4-(dimethylamino)-6- methoxy-1-oxoisoindolin-2- yl)piperidine-2,6-dione |
| 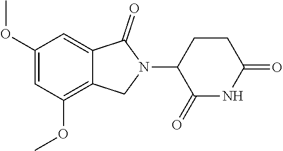 [SMILES: COc1cc(OC)c2c(c1)C(=O)N(C1CCC(=O)NC1=O)C2] | 3-(4,6-dimethoxy-1- oxoisoindolin-2-yl)piperidine- 2,6-dione |
| 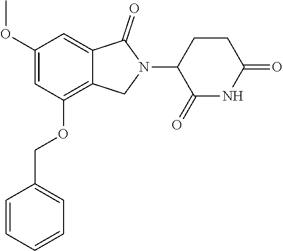 [SMILES: COc1cc(OCc2ccccc2)c2c(c1)C(=O)N(C1CCC(=O)NC1=O)C2] | 3-(4-(benzyloxy)-6-methoxy- 1-oxoisoindolin-2- yl)piperidine-2,6-dione |
| 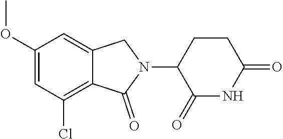 [SMILES: COc1cc(Cl)c2c(c1)CN(C1CCC(=O)NC1=O)C2=O] | 3-(7-chloro-5-methoxy-1- oxoisoindolin-2-yl)piperidine- 2,6-dione |
| 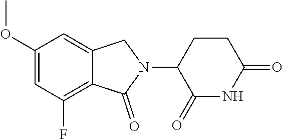 [SMILES: COc1cc(F)c2c(c1)CN(C1CCC(=O)NC1=O)C2=O] | 3-(7-fluoro-5-methoxy-1- oxoisoindolin-2-yl)piperidine- 2,6-dione |
| 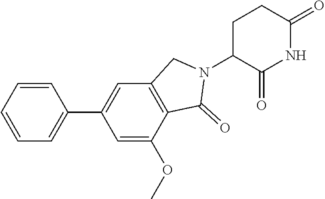 [SMILES: COc1cc(-c2ccccc2)cc2c1C(=O)N(C1CCC(=O)NC1=O)C2] | 3-(7-methoxy-1-oxo-5- phenylisoindolin-2- yl)piperidine-2,6-dione |
| 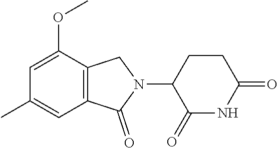 [SMILES: COc1cc(C)cc2c1CN(C1CCC(=O)NC1=O)C2=O] | 3-(4-methoxy-6-methyl-1- oxoisoindolin-2-yl)piperidine- 2,6-dione |
| 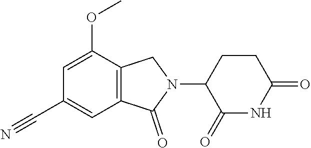 [SMILES: COc1cc(C#N)cc2c1CN(C1CCC(=O)NC1=O)C2=O] | 2-(2,6-dioxopiperidin-3-yl)-7- methoxy-3-oxoisoindoline-5- carbonitrile |
| 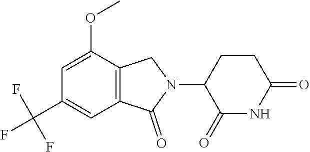 [SMILES: COc1cc(C(F)(F)F)cc2c1CN(C1CCC(=O)NC1=O)C2=O] | 3-(4-methoxy-1-oxo-6- (trifluoromethyl)isoindolin-2- yl)piperidine-2,6-dione |
| 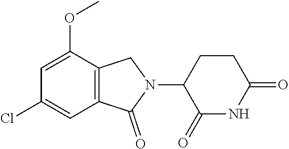 [SMILES: COc1cc(Cl)cc2c1CN(C1CCC(=O)NC1=O)C2=O] | 3-(6-chloro-4-methoxy-1- oxoisoindolin-2-yl)piperidine- 2,6-dione |
| 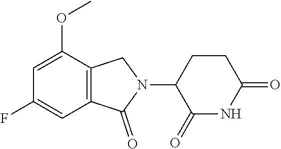 [SMILES: COc1cc(F)cc2c1CN(C1CCC(=O)NC1=O)C2=O] | 3-(6-fluoro-4-methoxy-1- oxoisoindolin-2-yl)piperidine- 2,6-dione |
| 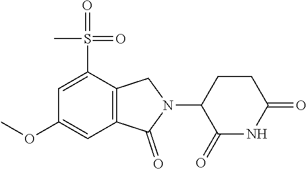 [SMILES: COc1cc2c(c(S(C)(=O)=O)c1)CN(C1CCC(=O)NC1=O)C2=O] | 3-(6-methoxy-4- (methylsulfonyl)-1- oxoisoindolin-2-yl)piperidine- 2,6-dione |
| 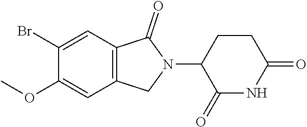 [SMILES: COc1cc2c(cc1Br)C(=O)N(C1CCC(=O)NC1=O)C2] | 3-(6-bromo-5-methoxy-1- oxoisoindolin-2-yl)piperidine- 2,6-dione |
| 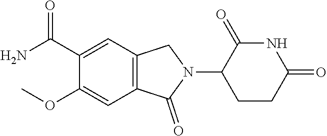 [SMILES: COc1cc2c(cc1C(N)=O)CN(C1CCC(=O)NC1=O)C2=O] | 2-(2,6-dioxopiperidin-3-yl)-6- methoxy-1-oxoisoindoline-5- carboxamide |
| 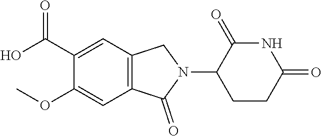 [SMILES: COc1cc2c(cc1C(=O)O)CN(C1CCC(=O)NC1=O)C2=O] | 2-(2,6-dioxopiperidin-3-yl)-6- methoxy-1-oxoisoindoline-5- carboxylic acid |
| 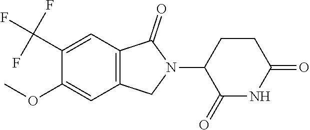 [SMILES: COc1cc2c(cc1C(F)(F)F)C(=O)N(C1CCC(=O)NC1=O)C2] | 3-(5-methoxy-1-oxo-6- (trifluromethyl)isoindolin-2- yl)piperidine-2,6-dione |
| 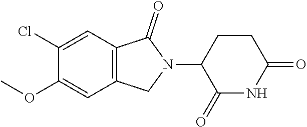 [SMILES: COc1cc2c(cc1Cl)C(=O)N(C1CCC(=O)NC1=O)C2] | 3-(6-chloro-5-methoxy-1- oxoisoindolin-2-yl)piperidine- 2,6-dione |
| 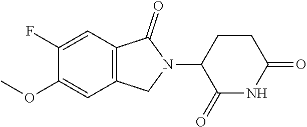 [SMILES: COc1cc2c(cc1F)C(=O)N(C1CCC(=O)NC1=O)C2] | 3-(6-fluoro-5-methoxy-1- oxoisoindolin-2-yl)piperidine- 2,6-dione |
| 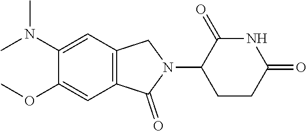 [SMILES: COc1cc2c(cc1N(C)C)CN(C1CCC(=O)NC1=O)C2=O] | 3-(5-(dimethylamino)-6- methoxy-1-oxoisoindolin-2- yl)piperidine-2,6-dione |
| 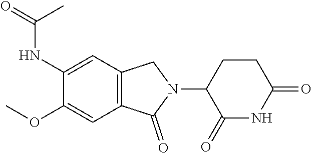 [SMILES: COc1cc2c(cc1NC(C)=O)CN(C1CCC(=O)NC1=O)C2=O] | N-(2-(2,6-dioxopiperidin-3- yl)-6-methoxy-1- oxoisoindolin-5-yl)acetamide |
| 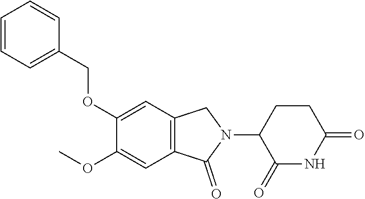 [SMILES: COc1cc2c(cc1OCc1ccccc1)CN(C1CCC(=O)NC1=O)C2=O] | 3-(5-(benzyloxy)-6-methoxy- 1-oxoisoindolin-2- yl)piperidine-2,6-dione |
| 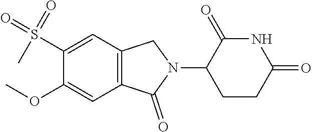 [SMILES: COc1cc2c(cc1S(C)(=O)=O)CN(C1CCC(=O)NC1=O)C2=O] | 3-(6-methoxy-5- (methylsulfonyl)-1- oxoisoindolin-2-yl)piperidine- 2,6-dione |
| 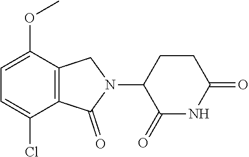 [SMILES: COc1ccc(Cl)c2c1CN(C1CCC(=O)NC1=O)C2=O] | 3-(7-chloro-4-methxoy-1- oxoisoindolin-2-yl)piperidine-2- 2,6-dione |
| 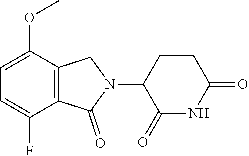 [SMILES: COc1ccc(F)c2c1CN(C1CCC(=O)NC1=O)C2=O] | 3-(7-fluoro-4-methoxy-1- oxoindoline-2-yl)piperi- [symbol] |
| 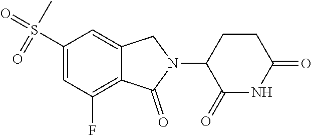 [SMILES: CS(=O)(=O)c1cc(F)c2c(c1)CN(C1CCC(=O)NC1=O)C2=O] | 3-(7-fluoro-5- (methylsulfonyl)-1- oxoisoindolin-2-yl)piperidine- 2,6-dione |
| 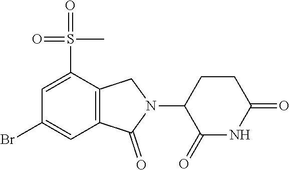 [SMILES: CS(=O)(=O)c1cc(Br)cc2c1CN(C1CCC(=O)NC1=O)C2=O] | 3-(6-bromo-4- (methylsulfonyl)-1- oxoisoindolin-2-yl)piperidine- 2,6-dione |
| 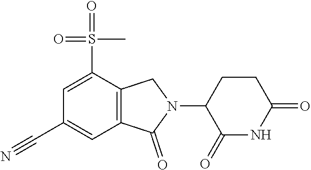 [SMILES: CS(=O)(=O)c1cc(C#N)cc2c1CN(C1CCC(=O)NC1=O)C2=O] | 2-(2,6-dioxopiperidin-3-yl)-7- (methylsulfonyl)-3- oxoisoindoline-5-carbonitrile |
| 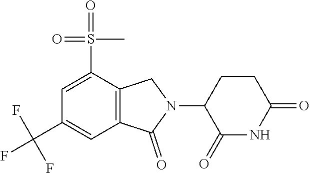 [SMILES: CS(=O)(=O)c1cc(C(F)(F)F)cc2c1CN(C1CCC(=O)NC1=O)C2=O] | 3-(4-(methylsulfonyl)-1-oxo- 6-(trifluoromethyl)isoindolin- 2-yl)piperidine-2,6-dione |
| 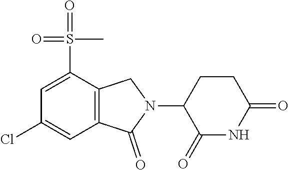 [SMILES: CS(=O)(=O)c1cc(Cl)cc2c1CN(C1CCC(=O)NC1=O)C2=O] | 3-(6-chloro-4- (methylsulfonyl)-1- oxoisoindolin-2-yl)piperidine- 2,6-dione |
| 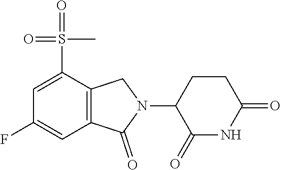 [SMILES: CS(=O)(=O)c1cc(F)cc2c1CN(C1CCC(=O)NC1=O)C2=O] | 3-(6-fluoro-4- (methylsulfonyl)-1- oxoisoindolin-2-yl)piperidine- 2,6-dione |
| 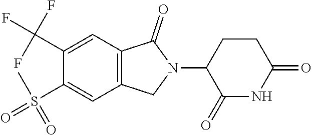 [SMILES: CS(=O)(=O)c1cc2c(cc1C(F)(F)F)C(=O)N(C1CCC(=O)NC1=O)C2] | 3-(5-(methyylsulfonyl)-1-oxo- 6-(trifluoromethyl)isoindolin- 2-yl)piperidine-2,6-dione |
| 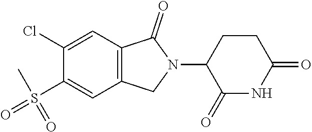 [SMILES: CS(=O)(=O)c1cc2c(cc1Cl)C(=O)N(C1CCC(=O)NC1=O)C2] | 3-(6-chloro-5- (methylsulfonyl)-1- oxoisoindolin-2-yl)piperidine- 2,6-dione |
| 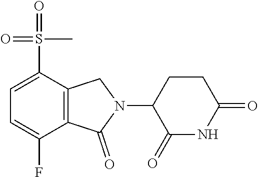 [SMILES: CS(=O)(=O)c1ccc(F)c2c1CN(C1CCC(=O)NC1=O)C2=O] | 3-(7-fluoro-4- (methylsulfonyl)-1- oxoisoindolin-2-yl)piperidine- 2,6-dione |
| 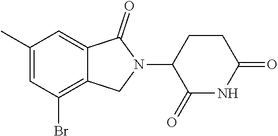 [SMILES: Cc1cc(Br)c2c(c1)C(=O)N(C1CCC(=O)NC1=O)C2] | 3-(4-bromo-6-methyl-1- oxoisoindolin-2-yl)piperidine- 2,6-dione |
| 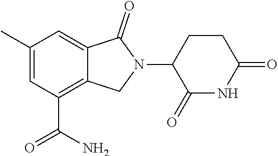 [SMILES: Cc1cc(C(N)=O)c2c(c1)C(=O)N(C1CCC(=O)NC1=O)C2] | 2-(2,6-dioxopiperidin-3-yl)-6- methyl-1-oxoisoindoline-4- carboxamide |
| 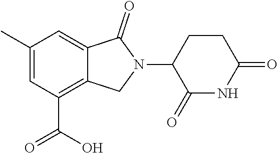 [SMILES: Cc1cc(C(=O)O)c2c(c1)C(=O)N(C1CCC(=O)NC1=O)C2] | 2-(2,6-dioxopiperidin-3-yl)-6- methyl-1-oxoisoindoline-4- carboxylic acid |
| 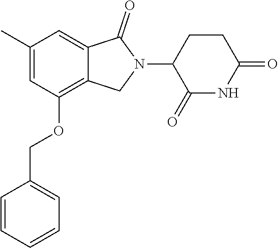 [SMILES: Cc1cc(OCc2ccccc2)c2c(c1)C(=O)N(C1CCC(=O)NC1=O)C2] | 3-(4-(benzyloxy)-6-methyl-1- oxoisoindolin-2-yl)piperidine- 2,6-dione |
| 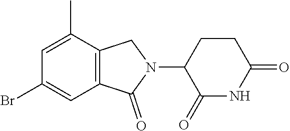 [SMILES: Cc1cc(Br)cc2c1CN(C1CCC(=O)NC1=O)C2=O] | 3-(6-bromo-4-methyl-1- oxoisoindolin-2-yl)piperidine- 2,6-dione |
| 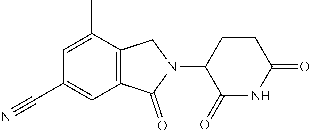 [SMILES: Cc1cc(C#N)cc2c1CN(C1CCC(=O)NC1=O)C2=O] | 2-(2,6-dioxopiperidin-3-yl)-7- methyl-3-oxoisoindoline-5- carbonitrile |
| 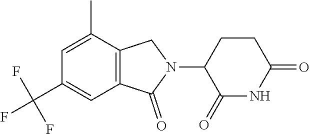 [SMILES: Cc1cc(C(F)(F)F)cc2c1CN(C1CCC(=O)NC1=O)C2=O] | 3-(4-methyl-1-oxo-6- (trifluoromethyl)isoindolin-2- yl)piperidine-2,6-dione |
| 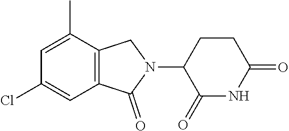 [SMILES: Cc1cc(Cl)cc2c1CN(C1CCC(=O)NC1=O)C2=O] | 3-(6-chloro-4-methyl-1- oxoisoindolin-2-yl)piperidine- 2,6-dione |
| 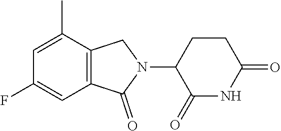 [SMILES: Cc1cc(F)cc2c1CN(C1CCC(=O)NC1=O)C2=O] | 3-(6-fluoro-4-methyl-1- oxoisoindolin-2-yl)piperidine- 2,6-dione |
| 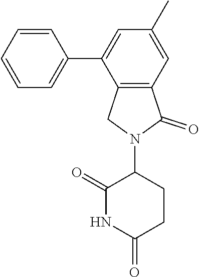 [SMILES: Cc1cc2c(c(-c3ccccc3)c1)CN(C1CCC(=O)NC1=O)C2=O] | 3-(6-methyl-1-oxo-4- phenylisoindolin-2- yl)piperidine-2,6-dione |
| 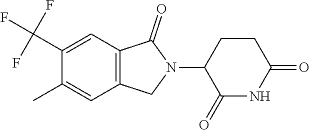 [SMILES: Cc1cc2c(cc1C(F)(F)F)C(=O)N(C1CCC(=O)NC1=O)C2] | 3-(5-methyl-1-oxo-6- (trifluroomethyl)isoindolin-2- yl)piperidine-2,6-dione |
| 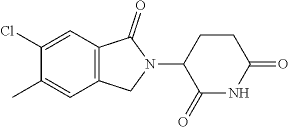 [SMILES: Cc1cc2c(cc1Cl)C(=O)N(C1CCC(=O)NC1=O)C2] | 3-(6-chloro-5-methyl-1- oxoisoindolin-2-yl)piperidine- 2,6-dione |
| 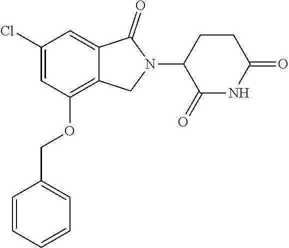 [SMILES: O=C1CCC(N2Cc3c(OCc4ccccc4)cc(Cl)cc3C2=O)C(=O)N1] | 3-(4-(benzyloxy)-6-chloro-1- oxoisoindolin-2-yl)piperidine- 2,6-dione |
| 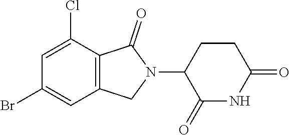 [SMILES: O=C1CCC(N2Cc3cc(Br)cc(Cl)c3C2=O)C(=O)N1] | 3-(5-bromo-7-chloro-1- oxoisoindolin-2-yl)piperidine- 2,6-dione |
| 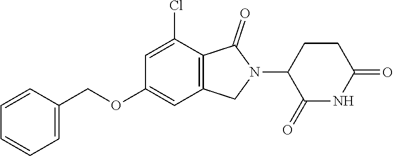 [SMILES: O=C1CCC(N2Cc3cc(OCc4ccccc4)cc(Cl)c3C2=O)C(=O)N1] | 3-(5-(benzyloxy)-7-chloro-1- oxoisoindolin-2-yl)piperidine- 2,6-dione |
| 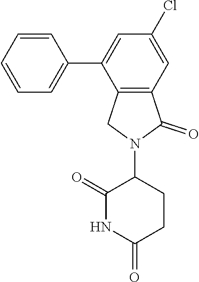 [SMILES: O=C1CCC(N2Cc3c(cc(Cl)cc3-c3ccccc3)C2=O)C(=O)N1] | 3-(6-chloro-1-oxo-4- phenylisoindolin-2- yl)piperidine-2,6-dione |
| 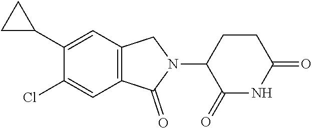 [SMILES: O=C1CCC(N2Cc3cc(C4CC4)c(Cl)cc3C2=O)C(=O)N1] | 3-(6-chloro-5-cyclopropyl-1- oxoisoindolin-2-yl)piperidine- 2,6-dione |
| 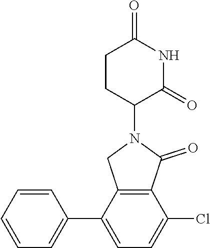 [SMILES: O=C1CCC(N2Cc3c(-c4ccccc4)ccc(Cl)c3C2=O)C(=O)N1] | 3-(7-chloro-1-oxo-4- phenylisoindolin-2- yl)piperidine-2,6-dione |
| 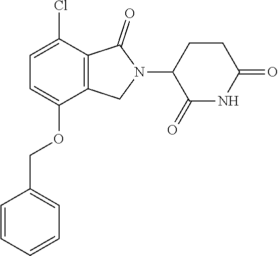 [SMILES: O=C1CCC(N2Cc3c(OCc4ccccc4)ccc(Cl)c3C2=O)C(=O)N1] | 3-(4-(benzyloxy)-7-chloro-1- oxoisoindolin-2-yl)piperidine- 2,6-dione |
| 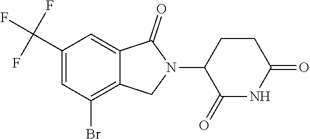 [SMILES: O=C1CCC(N2Cc3c(Br)cc(C(F)(F)F)cc3C2=O)C(=O)N1] | 3-(4-bromo-1-oxo-6- (trifluoromethyl)isoindolin-2- yl)piperidine-2,6-dione |
| 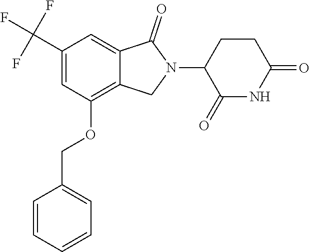 [SMILES: O=C1CCC(N2Cc3c(OCc4ccccc4)cc(C(F)(F)F)cc3C2=O)C(=O)N1] | 3-(4-(benzyloxy)-1-oxo-6- (trifluoromethyl)isoindolin-2- yl)piperidine-2,6-dione |
| 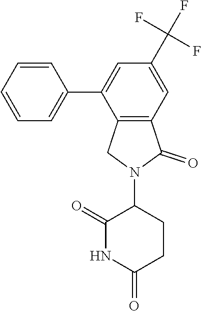 [SMILES: O=C1CCC(N2Cc3c(cc(C(F)(F)F)cc3-c3ccccc3)C2=O)C(=O)N1] | 3-(1-oxo-4-phenyl-6- (trifluoromethyl)isoindolin-2- yl)piperidine-2,6-dione |
| 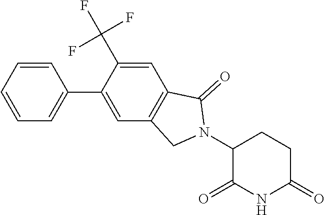 [SMILES: O=C1CCC(N2Cc3cc(-c4ccccc4)c(C(F)(F)F)cc3C2=O)C(=O)N1] | 3-(1-oxo-5-phenyl-6- (trifluoromethyl)isoindolin-2- yl)piperidine-2,6-dione |
| 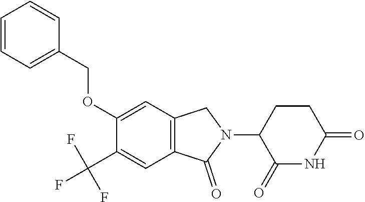 [SMILES: O=C1CCC(N2Cc3cc(OCc4ccccc4)c(C(F)(F)F)cc3C2=O)C(=O)N1] | 3-(5-(benzyloxy)-1-oxo-6- (trifluroomethyl)isoindolin-2- yl)piperidine-2,6-dione |
| 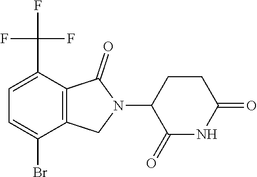 [SMILES: O=C1CCC(N2Cc3c(Br)ccc(C(F)(F)F)c3C2=O)C(=O)N1] | 3-(4-bromo-1-oxo-7- (trifluroomethyl)isoindolin-2- yl)piperidine-2,6-dione |
| 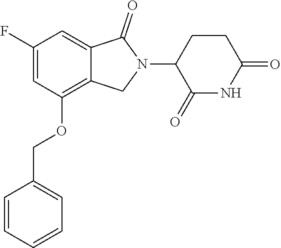 [SMILES: O=C1CCC(N2Cc3c(OCc4ccccc4)cc(F)cc3C2=O)C(=O)N1] | 3-(4-(benzyloxy)-6-fluoro-1- oxoisoindolin-2-yl)piperidine- 2,6-dione |
| 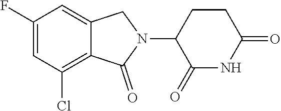 [SMILES: O=C1CCC(N2Cc3cc(F)cc(Cl)c3C2=O)C(=O)N1] | 3-(7-chloro-5-fluoro-1- oxoisoindolin-2-yl)piperidine- 2,6-dione |
| 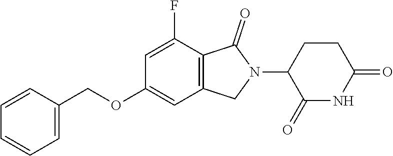 [SMILES: O=C1CCC(N2Cc3cc(OCc4ccccc4)cc(F)c3C2=O)C(=O)N1] | 3-(5-(benzyloxy)-7-fluoro-1- oxoisoindolin-2-yl)piperidine- 2,6-dione |
| 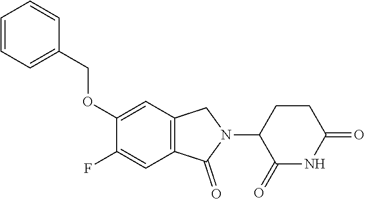 [SMILES: O=C1CCC(N2Cc3cc(OCc4ccccc4)c(F)cc3C2=O)C(=O)N1] | 3-(5-(benzyloxy)-6-fluoro-1- oxoisoindolin-2-yl)piperidine- 2,6-dione |
| 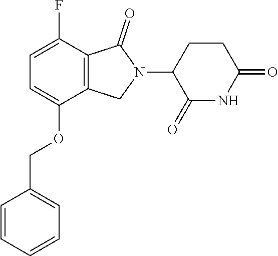 [SMILES: O=C1CCC(N2Cc3c(OCc4ccccc4)ccc(F)c3C2=O)C(=O)N1] | 3-(4-(benzyloxy)-7-fluoro-1- oxoisoindolin-2-yl)piperidine- 2,6-dione |
| 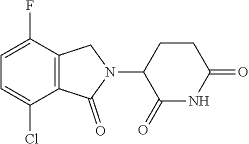 [SMILES: O=C1CCC(N2Cc3c(F)ccc(Cl)c3C2=O)C(=O)N1] | 3-(7-chloro-4-fluoro-1- oxoisoindolin-2-yl)piperidine- 2,6-dione |
| 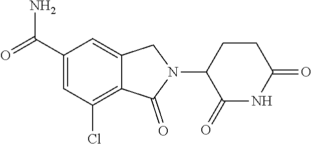 [SMILES: NC(=O)c1cc(Cl)c2c(c1)CN(C1CCC(=O)NC1=O)C2=O] | 7-chloro-2-(2,6- dioxopiperidin-3-yl)-1- oxoisoindoline-5-carboxamide |
| 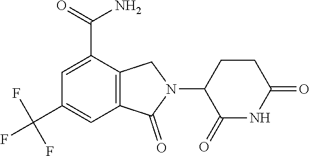 [SMILES: NC(=O)c1cc(C(F)(F)F)cc2c1CN(C1CCC(=O)NC1=O)C2=O] | 2-(2,6-dioxopiperidin-3-yl)-1- oxo-6- (trifluoromethyl)isoindoline-4- carboxamide |
| 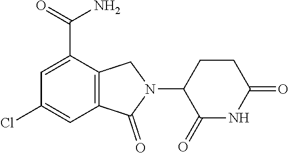 [SMILES: NC(=O)c1cc(Cl)cc2c1CN(C1CCC(=O)NC1=O)C2=O] | 6-chloro-2-(2,6- dioxopiperidin-3-yl)-1- oxoisoindoline-4-carboxamide |
| 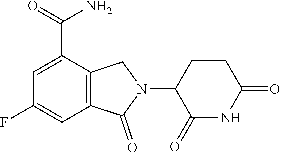 [SMILES: NC(=O)c1cc(F)cc2c1CN(C1CCC(=O)NC1=O)C2=O] | 2-(2,6-dioxopiperidin-3-yl)-6- fluoro-1-oxoisoindoline-4- carboxamide |
| 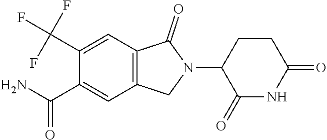 [SMILES: NC(=O)c1cc2c(cc1C(F)(F)F)C(=O)N(C1CCC(=O)NC1=O)C2] | 2-(2,6-dioxopiperidin-3-yl)-1- oxo-6- (trifluoromethyl)isoindoline-5- carboxamide |
| 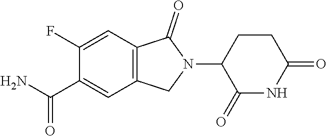 [SMILES: NC(=O)c1cc2c(cc1F)C(=O)N(C1CCC(=O)NC1=O)C2] | 2-(2,6-dioxopiperidin-3-yl)-6- fluoro-1-oxoisoindoline-5- carboxamide |
| 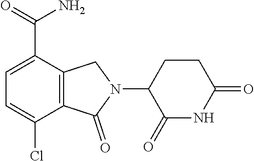 [SMILES: NC(=O)c1ccc(Cl)c2c1CN(C1CCC(=O)NC1=O)C2=O] | 7-chloro-2-(2,6- dioxopiperidin-3-yl)-1- oxoisoindoline-4-carboxamide |
| 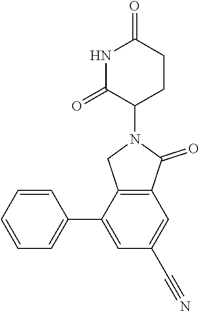 [SMILES: N#Cc1cc2c(c(-c3ccccc3)c1)CN(C1CCC(=O)NC1=O)C2=O] | 2-(2,6-dioxopiperidin-3-yl)-3- oxo-7-phenylisoindoline-5- carbonitrile |
| 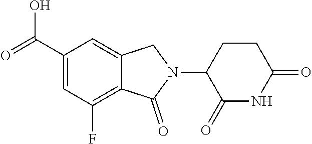 [SMILES: O=C1CCC(N2Cc3cc(C(=O)O)cc(F)c3C2=O)C(=O)N1] | 2-(2,6-dioxopiperidin-3-yl)-7- fluoro-1-oxoisoindoline-5- carboxylic acid |
| 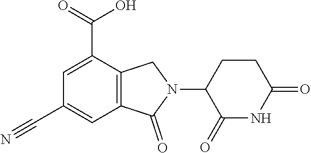 [SMILES: N#Cc1cc(C(=O)O)c2c(c1)C(=O)N(C1CCC(=O)NC1=O)C2] | 6-cyano-2-(2,6- dioxopiperidin-3-yl)-1- oxoisoindoline-4-carboxylic acid |
| 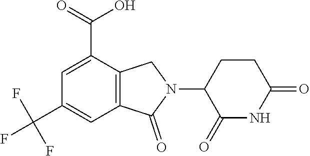 [SMILES: O=C1CCC(N2Cc3c(C(=O)O)cc(C(F)(F)F)cc3C2=O)C(=O)N1] | 2-(2,6-dioxopiperidin-3-yl)-1- oxo-6- (trifluoromethyl)isoindoline-4- carboxylic acid |
| 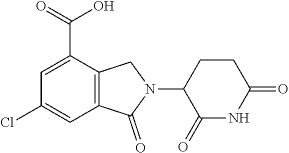 [SMILES: O=C1CCC(N2Cc3c(C(=O)O)cc(Cl)cc3C2=O)C(=O)N1] | 6-chloro-2-(2,6- dioxopiperidin-3-yl)-1- oxoisoindoline-4-carboxylic acid |
| 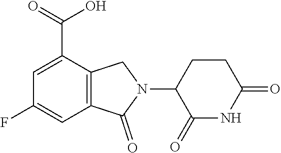 [SMILES: O=C1CCC(N2Cc3c(C(=O)O)cc(F)cc3C2=O)C(=O)N1] | 2-(2,6-dioxopiperidin-3-yl)-6- fluoro-1-oxoisoindoline-4- carboxylic acid |
| 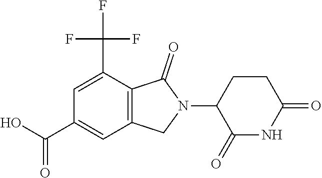 [SMILES: O=C1CCC(N2Cc3cc(C(=O)O)cc(C(F)(F)F)c3C2=O)C(=O)N1] | 2-(2,6-dioxopiperidin-3-yl)-1- oxo-7- (trifluoromethyl)isoindoline-5- carboxylic acid |
| 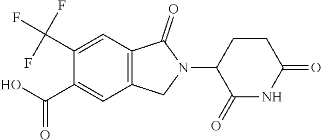 [SMILES: O=C1CCC(N2Cc3cc(C(=O)O)c(C(F)(F)F)cc3C2=O)C(=O)N1] | 2-(2,6-dioxopiperidin-3-yl)-1- oxo-6- (trifluoromethyl)isoindoline-5- carboxylic acid |
| 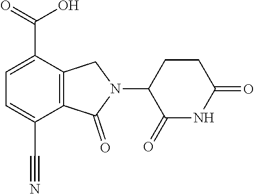 [SMILES: N#Cc1ccc(C(=O)O)c2c1C(=O)N(C1CCC(=O)NC1=O)C2] | 7-cyano-2-(2,6- dioxopiperidin-3-yl)-1- oxoisoindoline-4-carboxylic acid |
| 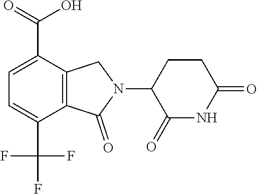 [SMILES: O=C1CCC(N2Cc3c(C(=O)O)ccc(C(F)(F)F)c3C2=O)C(=O)N1] | 2-(2,6-dioxopiperidin-3-yl)-1- oxo-7- (trifluoromethyl)isoindoline-4- carboxylic acid |
| 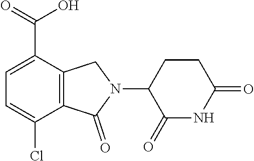 [SMILES: O=C1CCC(N2Cc3c(C(=O)O)ccc(Cl)c3C2=O)C(=O)N1] | 7-chloro-2-(2,6- dioxopiperidin-3-yl)-1- oxoisoindoline-4-carboxylic aicd |
| 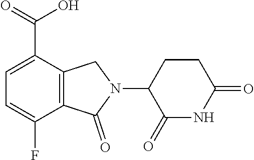 [SMILES: O=C1CCC(N2Cc3c(C(=O)O)ccc(F)c3C2=O)C(=O)N1] | 2-(2,6-dioxopiperidin-3-yl)-7- fluoro-1-oxoisoindoline-4- carboxylic acid |

[symbol]

indicates data missing or illegible when filed

TABLE 2

| Chemical Structure | Name |
|---|---|
| 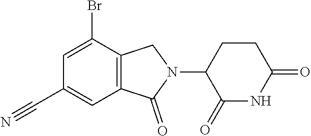 [SMILES: N#Cc1cc(Br)c2c(c1)C(=O)N(C1CCC(=O)NC1=O)C2] | 7-bromo-2-(2,6-dioxopiperidin-3-yl)-3- oxoisoindoline-5-carbonitrile |
| 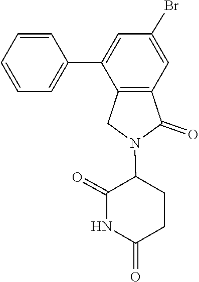 [SMILES: O=C1CCC(N2Cc3c(cc(Br)cc3-c3ccccc3)C2=O)C(=O)N1] | 3-(6-bromo-1-oxo-4-phenylisoindolin-2- yl)piperidine-2,6-dione |
| 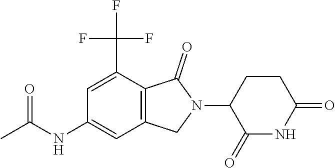 [SMILES: CC(=O)Nc1cc2c(c(C(F)(F)F)c1)C(=O)N(C1CCC(=O)NC1=O)C2] | N-(2-(2,6-dioxopiperidin-3-yl)-1-oxo-7- (trifluoromethyl)isoindolin-5-yl)acetamide |
| 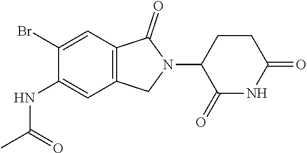 [SMILES: CC(=O)Nc1cc2c(cc1Br)C(=O)N(C1CCC(=O)NC1=O)C2] | N-(6-bromo-2-(2,6-dioxopiperidin-3-yl)-1- oxoisoindolin-5-yl)acetamide |
| 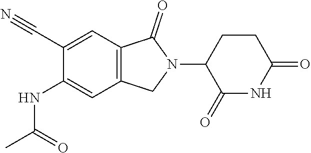 [SMILES: CC(=O)Nc1cc2c(cc1C#N)C(=O)N(C1CCC(=O)NC1=O)C2] | N-(6-cyano-2-(2,6-dioxopiperidin-3-yl)-1- oxoisoindolin-5-yl)acetamide |
| 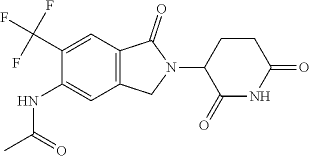 [SMILES: CC(=O)Nc1cc2c(cc1C(F)(F)F)C(=O)N(C1CCC(=O)NC1=O)C2] | N-(2-(2,6-dioxopiperidin-3-yl)-1-oxo-6- (trifluoromethyl)isoindolin-5-yl)acetamide |
| 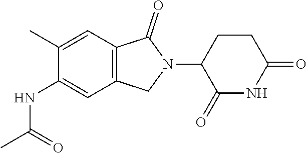 [SMILES: CC(=O)Nc1cc2c(cc1C)C(=O)N(C1CCC(=O)NC1=O)C2] | N-(2-(2,6-dioxopiperidin-3-yl)-6-methyl-1- oxoisoindolin-5-yl)acetamide |
| 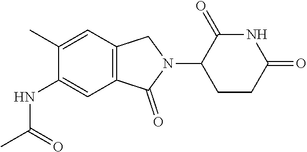 [SMILES: CC(=O)Nc1cc2c(cc1C)CN(C1CCC(=O)NC1=O)C2=O] | N-(2-(2,6-dioxopiperidin-3-yl)-6-methyl-3- oxoisoindolin-5-yl)acetamide |
| 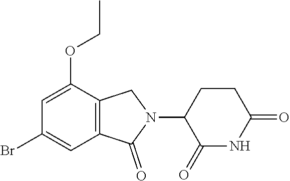 [SMILES: CCOc1cc(Br)cc2c1CN(C1CCC(=O)NC1=O)C2=O] | 3-(6-bromo-4-ethoxy-1-oxoisoindolin-2- yl)piperidine-2,6-dione |
| 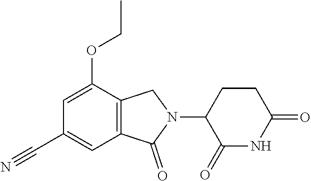 [SMILES: CCOc1cc(C#N)cc2c1CN(C1CCC(=O)NC1=O)C2=O] | 2-(2,6-dioxopiperidin-3-yl)-7-ethoxy-3- oxoisoindoline-5-carbonitrile |
| 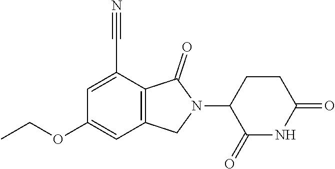 [SMILES: CCOc1cc(C#N)c2c(c1)CN(C1CCC(=O)NC1=O)C2=O] | 2-(2,6-dioxopiperidin-3-yl)-6-ethoxy-3- oxoisoindoline-4-carbonitrile |
| 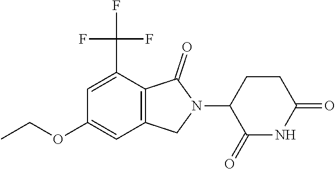 [SMILES: CCOc1cc2c(c(C(F)(F)F)c1)C(=O)N(C1CCC(=O)NC1=O)C2] | 3-(5-ethoxy-1-oxo-7- (trifluoromethyl)isoindolin-2-yl)piperidine- 2,6-dione |
| 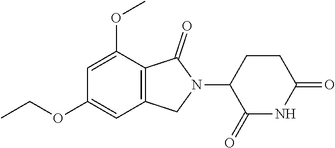 [SMILES: CCOc1cc2c(c(OC)c1)C(=O)N(C1CCC(=O)NC1=O)C2] | 3-(5-ethoxy-7-methoxy-1-oxoisoindolin-2- yl)piperidine-2,6-dione |
| 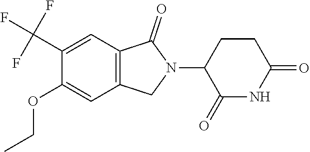 [SMILES: CCOc1cc2c(cc1C(F)(F)F)C(=O)N(C1CCC(=O)NC1=O)C2] | 3-(5-ethoxy-1-oxo-6- (trifluoromethyl)isoindolin-2-yl)piperidine- 2,6-dione |
| 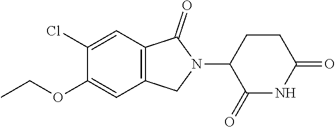 [SMILES: CCOc1cc2c(cc1Cl)C(=O)N(C1CCC(=O)NC1=O)C2] | 3-(6-chloro-5-ethoxy-1-oxoisoindolin-2- yl)piperidine-2,6-dione |
| 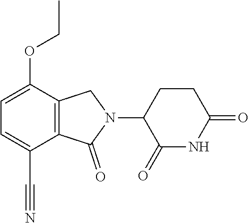 [SMILES: CCOc1ccc(C#N)c2c1CN(C1CCC(=O)NC1=O)C2=O] | 2-(2,6-dioxopiperidin-3-yl)-7-ethoxy-3- oxoisoindoline-4-carbonitrile |
| 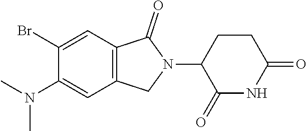 [SMILES: CN(C)c1cc2c(cc1Br)C(=O)N(C1CCC(=O)NC1=O)C2] | 3-(6-bromo-5-(dimethylamino)-1- oxoisoindolin-2-yl)piperidine-2,6-dione |
| 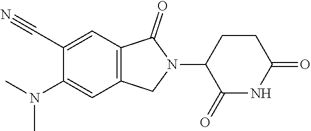 [SMILES: CN(C)c1cc2c(cc1C#N)C(=O)N(C1CCC(=O)NC1=O)C2] | 6-(dimethylamino)-2-(2,6-dioxopiperidin- 3-yl)-3-oxoisoindoline-5-carbonitrile |
| 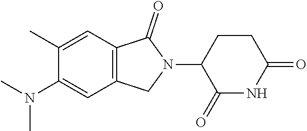 [SMILES: Cc1cc2c(cc1N(C)C)CN(C1CCC(=O)NC1=O)C2=O] | 3-(5-(dimethylamino)-6-methyl-1- oxoisoindolin-2-yl)piperidine-2,6-dione |
| 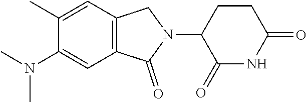 [SMILES: Cc1cc2c(cc1N(C)C)C(=O)N(C1CCC(=O)NC1=O)C2] | 3-(6-(dimethylamino)-5-methyl-1- oxoisoindolin-2-yl)piperidine-2,6-dione |
| 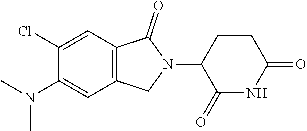 [SMILES: CN(C)c1cc2c(cc1Cl)C(=O)N(C1CCC(=O)NC1=O)C2] | 3-(6-chloro-5-(dimethylamino)-1- oxoisoindolin-2-yl)piperidine-2,6-dione |
| 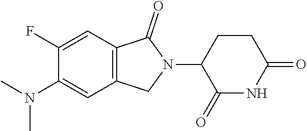 [SMILES: CN(C)c1cc2c(cc1F)C(=O)N(C1CCC(=O)NC1=O)C2] | 3-(5-(dimethylamino)-6-fluoro-1- oxoisoindolin-2-yl)piperidine-2,6-dione |
| 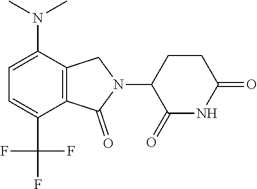 [SMILES: CN(C)c1ccc(C(F)(F)F)c2c1CN(C1CCC(=O)NC1=O)C2=O] | 3-(4-(dimethylamino)-1-oxo-7- (trifluoromethyl)isoindolin-2-yl)piperidine- 2,6-dione |
| 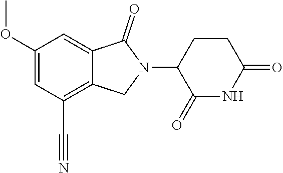 [SMILES: COc1cc(C#N)c2c(c1)C(=O)N(C1CCC(=O)NC1=O)C2] | 2-(2,6-dioxopiperidin-3-yl)-6-methoxy-1- oxoisoindoline-4-carbonitrile |
| 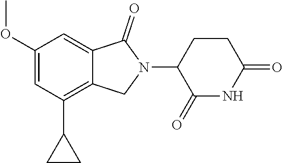 [SMILES: COc1cc2c(c(C3CC3)c1)CN(C1CCC(=O)NC1=O)C2=O] | 3-(4-cyclopropyl-6-methoxy-1- oxoisoindolin-2-yl)piperidine-2,6-dione |
| 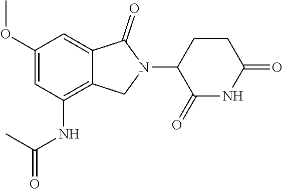 [SMILES: COc1cc(NC(C)=O)c2c(c1)C(=O)N(C1CCC(=O)NC1=O)C2] | N-(2-(2,6-dioxopiperidin-3-yl)-6-methoxy- 1-oxoisoindolin-4-yl)acetamide |
| 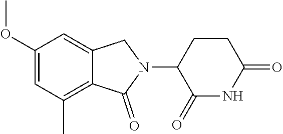 [SMILES: COc1cc(C)c2c(c1)CN(C1CCC(=O)NC1=O)C2=O] | 3-(5-methoxy-7-methyl-1-oxoisoindolin-2- yl)piperidine-2,6-dione |
| 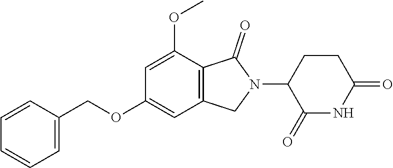 [SMILES: COc1cc(OCc2ccccc2)cc2c1C(=O)N(C1CCC(=O)NC1=O)C2] | 3-(5-(benzyloxy)-7-methoxy-1- oxoisoindolin-2-yl)piperidine-2,6-dione |
| 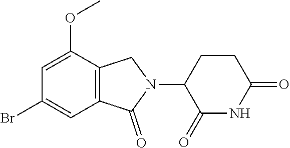 [SMILES: COc1cc(Br)cc2c1CN(C1CCC(=O)NC1=O)C2=O] | 3-(6-bromo-4-methoxy-1-oxoisoindolin-2- yl)piperidine-2,6-dione |
| 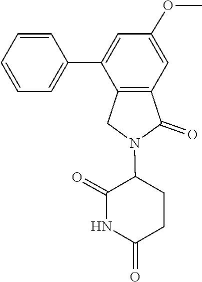 [SMILES: COc1cc2c(c(-c3ccccc3)c1)CN(C1CCC(=O)NC1=O)C2=O] | 3-(6-methoxy-1-oxo-4-phenylisoindolin-2- yl)piperidine-2,6-dione |
| 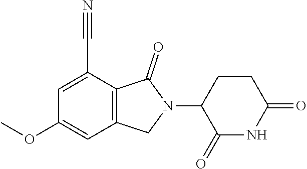 [SMILES: COc1cc(C#N)c2c(c1)CN(C1CCC(=O)NC1=O)C2=O] | 2-(2,6-dioxopiperidin-3-yl)-6-methoxy-3- oxoisoindoline-4-carbonitrile |
| 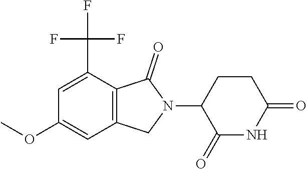 [SMILES: COc1cc2c(c(C(F)(F)F)c1)C(=O)N(C1CCC(=O)NC1=O)C2] | 3-(5-methoxy-1-oxo-7- (trifluoromethyl)isoindolin-2-yl)piperidine- 2,6-dione |
| 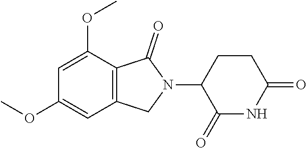 [SMILES: COc1cc2c(c(OC)c1)C(=O)N(C1CCC(=O)NC1=O)C2] | 3-(5,7-dimethoxy-1-oxoisoindolin-2- yl)piperidine-2,6-dione |
| 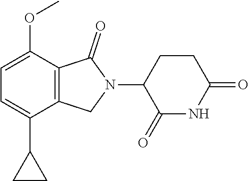 [SMILES: COc1ccc(C2CC2)c2c1C(=O)N(C1CCC(=O)NC1=O)C2] | 3-(4-cyclopropyl-7-methoxy-1- oxoisoindolin-2-yl)piperidine-2,6-dione |
| 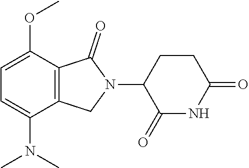 [SMILES: COc1ccc(N(C)C)c2c1C(=O)N(C1CCC(=O)NC1=O)C2] | 3-(4-(dimethylamino)-7-methoxy-1- oxoisoindolin-2-yl)piperidine-2,6-dione |
| 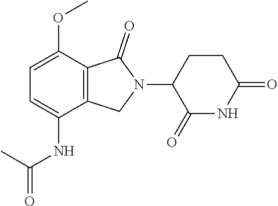 [SMILES: COc1ccc(NC(C)=O)c2c1C(=O)N(C1CCC(=O)NC1=O)C2] | N-(2-(2,6-dioxopiperidin-3-yl)-7-methoxy- 1-oxoisoindolin-4-yl)acetamide |
| 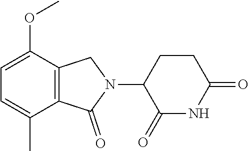 [SMILES: COc1ccc(C)c2c1CN(C1CCC(=O)NC1=O)C2=O] | 3-(4-methoxy-7-methyl-1-oxoisoindolin-2- yl)piperidine-2,6-dione |
| 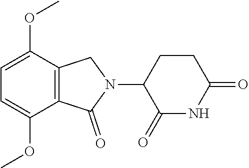 [SMILES: COc1ccc(OC)c2c1CN(C1CCC(=O)NC1=O)C2=O] | 3-(4,7-dimethoxy-1-oxoisoindolin-2- yl)piperidine-2,6-dione |
| 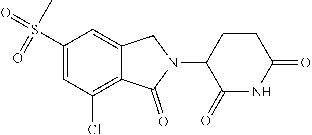 [SMILES: CS(=O)(=O)c1cc(Cl)c2c(c1)CN(C1CCC(=O)NC1=O)C2=O] | 3-(7-chloro-5-(methylsulfonyl)-1- oxoisoindolin-2-yl)piperidine-2,6-dione |
| 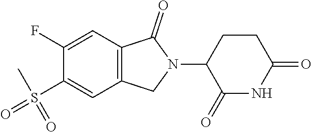 [SMILES: CS(=O)(=O)c1cc2c(cc1F)C(=O)N(C1CCC(=O)NC1=O)C2] | 3-(6-fluoro-5-(methylsulfonyl)-1- oxoisoindolin-2-yl)piperidine-2,6-dione |
| 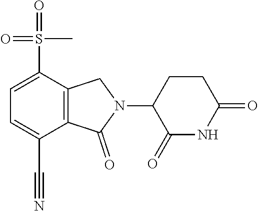 [SMILES: CS(=O)(=O)c1ccc(C#N)c2c1CN(C1CCC(=O)NC1=O)C2=O] | 2-(2,6-dioxopiperidin-3-yl)-7- (methylsulfonyl)-3-oxoisoindoline-4- carbonitrile |
| 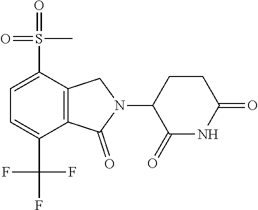 [SMILES: CS(=O)(=O)c1ccc(C(F)(F)F)c2c1CN(C1CCC(=O)NC1=O)C2=O] | 3-(4-(methylsulfonyl)-1-oxo-7- (trifluoromethyl)isoindolin-2-yl)piperidine- 2,6-dione |
| 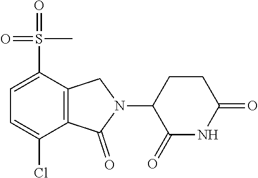 [SMILES: CS(=O)(=O)c1ccc(Cl)c2c1CN(C1CCC(=O)NC1=O)C2=O] | 3-(7-chloro-4-(methylsulfonyl)-1- oxoisoindolin-2-yl)piperidine-2,6-dione |
| 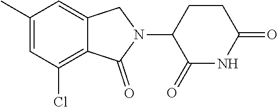 [SMILES: Cc1cc(Cl)c2c(c1)CN(C1CCC(=O)NC1=O)C2=O] | 3-(7-chloro-5-methyl-1-oxoisoindolin-2- yl)piperidine-2,6-dione |
| 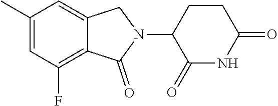 [SMILES: Cc1cc(F)c2c(c1)CN(C1CCC(=O)NC1=O)C2=O] | 3-(7-fluoro-5-methyl-1-oxoisoindolin-2- yl)piperidine-2,6-dione |
| 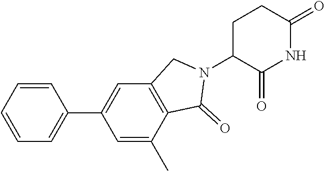 [SMILES: Cc1cc(-c2ccccc2)cc2c1C(=O)N(C1CCC(=O)NC1=O)C2] | 3-(7-methyl-1-oxo-5-phenylisoindolin-2- yl)piperidine-2,6-dione |
| 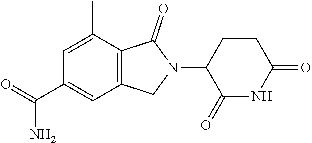 [SMILES: Cc1cc(C(N)=O)cc2c1C(=O)N(C1CCC(=O)NC1=O)C2] | 2-(2,6-dioxopiperidin-3-yl)-7-methyl-1- oxoisoindoline-5-carboxamide |
| 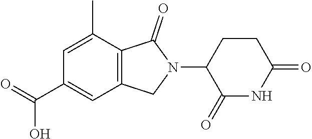 [SMILES: Cc1cc(C(=O)O)cc2c1C(=O)N(C1CCC(=O)NC1=O)C2] | 2-(2,6-dioxopiperidin-3-yl)-7-methyl-1- oxoisoindoline-5-carboxylic acid |
| 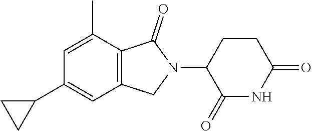 [SMILES: Cc1cc(C2CC2)cc2c1C(=O)N(C1CCC(=O)NC1=O)C2] | 3-(5-cyclopropyl-7-methyl-1- oxoisoindolin-2-yl)piperidine-2,6-dione |
| 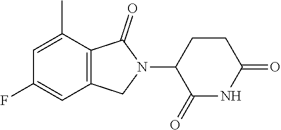 [SMILES: Cc1cc(F)cc2c1C(=O)N(C1CCC(=O)NC1=O)C2] | 3-(5-fluoro-7-methyl-1-oxoisoindolin-2- yl)piperidine-2,6-dione |
| 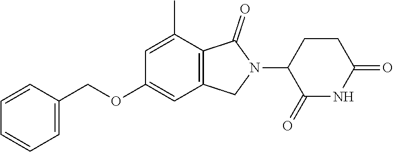 [SMILES: Cc1cc(OCc2ccccc2)cc2c1C(=O)N(C1CCC(=O)NC1=O)C2] | 3-(5-(benzyloxy)-7-methyl-1- oxoisoindolin-2-yl)piperidine-2,6-dione |
| 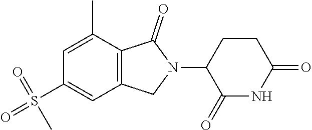 [SMILES: Cc1cc(S(C)(=O)=O)cc2c1C(=O)N(C1CCC(=O)NC1=O)C2] | 3-(7-methyl-5-(methylsulfonyl)-1- oxoisoindolin-2-yl)piperidine-2,6-dione |
| 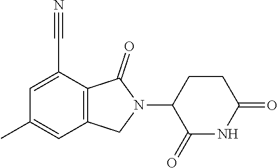 [SMILES: Cc1cc(C#N)c2c(c1)CN(C1CCC(=O)NC1=O)C2=O] | 2-(2,6-dioxopiperidin-3-yl)-6-methyl-3- oxoisoindoline-4-carbonitrile |
| 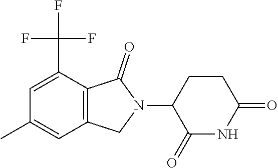 [SMILES: Cc1cc2c(c(C(F)(F)F)c1)C(=O)N(C1CCC(=O)NC1=O)C2] | 3-(5-methyl-1-oxo-7- (trifluoromethyl)isoindolin-2-yl)piperidine- 2,6-dione |
| 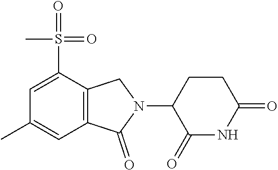 [SMILES: Cc1cc2c(c(S(C)(=O)=O)c1)CN(C1CCC(=O)NC1=O)C2=O] | 3-(6-methyl-4-(methylsulfonyl)-1- oxoisoindolin-2-yl)piperidine-2,6-dione |
| 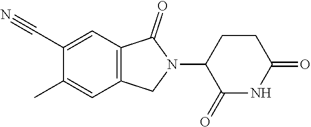 [SMILES: Cc1cc2c(cc1C#N)C(=O)N(C1CCC(=O)NC1=O)C2] | 2-(2,6-dioxopiperidin-3-yl)-6-methyl-3- oxoisoindoline-5-carbonitrile |
| 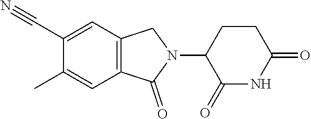 [SMILES: Cc1cc2c(cc1C#N)CN(C1CCC(=O)NC1=O)C2=O] | 2-(2,6-dioxopiperidin-3-yl)-6-methyl-1- oxoisoindoline-5-carbonitrile |
| 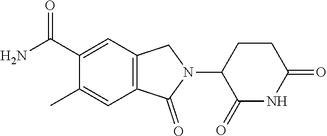 [SMILES: Cc1cc2c(cc1C(N)=O)CN(C1CCC(=O)NC1=O)C2=O] | 2-(2,6-dioxopiperidin-3-yl)-6-methyl-1- oxoisoindoline-5-carboxamide |
| 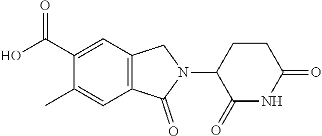 [SMILES: Cc1cc2c(cc1C(=O)O)CN(C1CCC(=O)NC1=O)C2=O] | 2-(2,6-dioxopiperidin-3-yl)-6-methyl-1- oxoisoindoline-5-carboxylic acid |
| 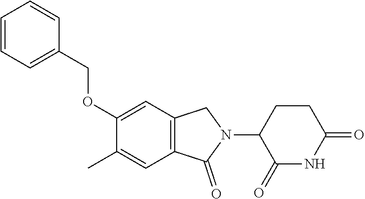 [SMILES: Cc1cc2c(cc1OCc1ccccc1)CN(C1CCC(=O)NC1=O)C2=O] | 3-(5-(benzyloxy)-6-methyl-1- oxoisoindolin-2-yl)piperidine-2,6-dione |
| 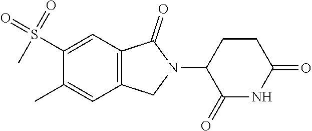 [SMILES: Cc1cc2c(cc1S(C)(=O)=O)C(=O)N(C1CCC(=O)NC1=O)C2] | 3-(5-methyl-6-(methylsulfonyl)-1- oxoisoindolin-2-yl)piperidine-2,6-dione |
| 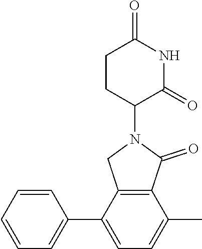 [SMILES: Cc1ccc(-c2ccccc2)c2c1C(=O)N(C1CCC(=O)NC1=O)C2] | 3-(7-methyl-1-oxo-4-phenylisoindolin-2- yl)piperidine-2,6-dione |
| 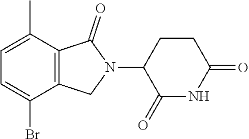 [SMILES: Cc1ccc(Br)c2c1C(=O)N(C1CCC(=O)NC1=O)C2] | 3-(4-bromo-7-methyl-1-oxoisoindolin-2- yl)piperidine-2,6-dione |
| 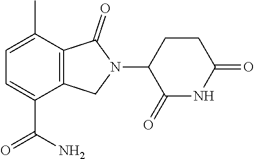 [SMILES: Cc1ccc(C(N)=O)c2c1C(=O)N(C1CCC(=O)NC1=O)C2] | 2-(2,6-dioxopiperidin-3-yl)-7-methyl-1- oxoisoindoline-4-carboxamide |
| 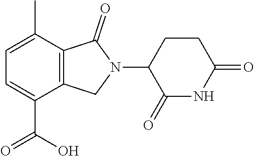 [SMILES: Cc1ccc(C(=O)O)c2c1C(=O)N(C1CCC(=O)NC1=O)C2] | 2-(2,6-dioxopiperidin-3-yl)-7-methyl-1- oxoisoindoline-4-carboxylic acid |
| 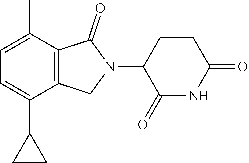 [SMILES: Cc1ccc(C2CC2)c2c1C(=O)N(C1CCC(=O)NC1=O)C2] | 3-(4-cyclopropyl-7-methyl-1- oxoisoindolin-2-yl)piperidine-2,6-dione |
| 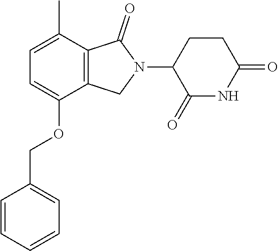 [SMILES: Cc1ccc(OCc2ccccc2)c2c1C(=O)N(C1CCC(=O)NC1=O)C2] | 3-(4-(benzyloxy)-7-methyl-1- oxoisoindolin-2-yl)piperidine-2,6-dione |
| 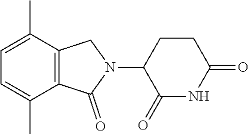 [SMILES: Cc1ccc(C)c2c1CN(C1CCC(=O)NC1=O)C2=O] | 3-(4,7-dimethyl-1-oxoisoindolin-2- yl)piperidine-2,6-dione |
| 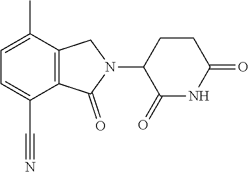 [SMILES: Cc1ccc(C#N)c2c1CN(C1CCC(=O)NC1=O)C2=O] | 2-(2,6-dioxopiperidin-3-yl)-7-methyl-3- oxoisoindoline-4-carbonitrile |
| 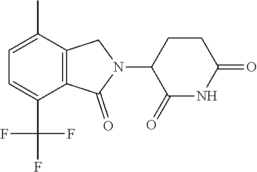 [SMILES: Cc1ccc(C(F)(F)F)c2c1CN(C1CCC(=O)NC1=O)C2=O] | 3-(4-methyl-1-oxo-7- (trifluoromethyl)isoindolin-2-yl)piperidine- 2,6-dione |
| 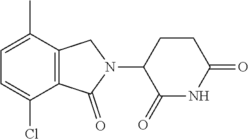 [SMILES: Cc1ccc(Cl)c2c1CN(C1CCC(=O)NC1=O)C2=O] | 3-(7-chloro-4-methyl-1-oxoisoindolin-2- yl)piperidine-2,6-dione |
| 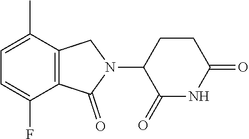 [SMILES: Cc1ccc(F)c2c1CN(C1CCC(=O)NC1=O)C2=O] | 3-(7-fluoro-4-methyl-1-oxoisoindolin-2- yl)piperidine-2,6-dione |
| 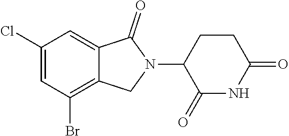 [SMILES: O=C1CCC(N2Cc3c(Br)cc(Cl)cc3C2=O)C(=O)N1] | 3-(4-bromo-6-chloro-1-oxoisoindolin-2- yl)piperidine-2,6-dione |
| 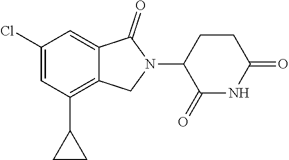 [SMILES: O=C1CCC(N2Cc3c(cc(Cl)cc3C3CC3)C2=O)C(=O)N1] | 3-(6-chloro-4-cyclopropyl-1-oxoisoindolin- 2-yl)piperidine-2,6-dione |
| 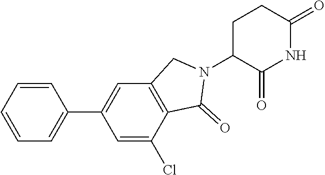 [SMILES: O=C1CCC(N2Cc3cc(-c4ccccc4)cc(Cl)c3C2=O)C(=O)N1] | 3-(7-chloro-1-oxo-5-phenylisoindolin-2- yl)piperidine-2,6-dione |
| 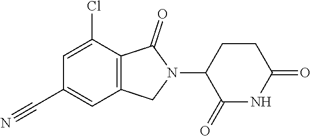 [SMILES: N#Cc1cc(Cl)c2c(c1)CN(C1CCC(=O)NC1=O)C2=O] | 7-chloro-2-(2,6-dioxopiperidin-3-yl)-1- oxoisoindoline-5-carbonitrile |
| 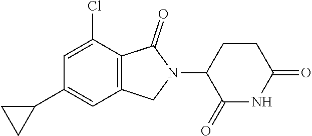 [SMILES: O=C1CCC(N2Cc3cc(C4CC4)cc(Cl)c3C2=O)C(=O)N1] | 3-(7-chloro-5-cyclopropyl-1-oxoisoindolin- 2-yl)piperidine-2,6-dione |
| 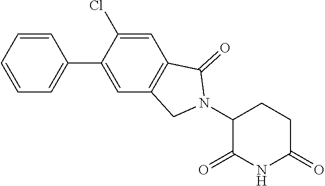 [SMILES: O=C1CCC(N2Cc3cc(-c4ccccc4)c(Cl)cc3C2=O)C(=O)N1] | 3-(6-chloro-1-oxo-5-phenylisoindolin-2- yl)piperidine-2,6-dione |
| 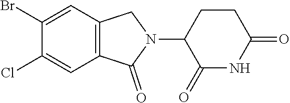 [SMILES: O=C1CCC(N2Cc3cc(Br)c(Cl)cc3C2=O)C(=O)N1] | 3-(5-bromo-6-chloro-1-oxoisoindolin-2- yl)piperidine-2,6-dione |
| 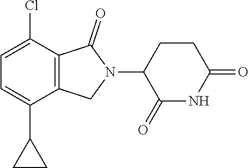 [SMILES: O=C1CCC(N2Cc3c(C4CC4)ccc(Cl)c3C2=O)C(=O)N1] | 3-(7-chloro-4-cyclopropyl-1-oxoisoindolin- 2-yl)piperidine-2,6-dione |
| 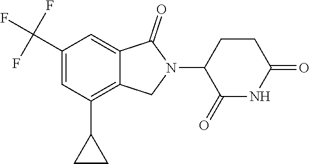 [SMILES: O=C1CCC(N2Cc3c(cc(C(F)(F)F)cc3C3CC3)C2=O)C(=O)N1] | 3-(4-cyclopropyl-1-oxo-6- (trifluoromethyl)isoindolin-2-yl)piperidine- 2,6-dione |
| 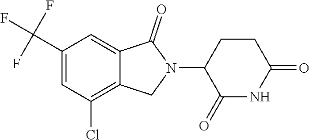 [SMILES: O=C1CCC(N2Cc3c(Cl)cc(C(F)(F)F)cc3C2=O)C(=O)N1] | 3-(4-chloro-1-oxo-6- (trifluoromethyl)isoindolin-2-yl)piperidine- 2,6-dione |
| 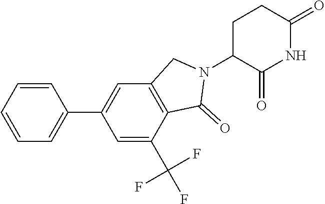 [SMILES: O=C1CCC(N2Cc3cc(-c4ccccc4)cc(C(F)(F)F)c3C2=O)C(=O)N1] | 3-(1-oxo-5-phenyl-7- (trifluoromethyl)isoindolin-2-yl)piperidine- 2,6-dione |
| 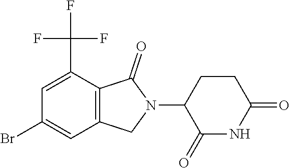 [SMILES: O=C1CCC(N2Cc3cc(Br)cc(C(F)(F)F)c3C2=O)C(=O)N1] | 3-(5-bromo-1-oxo-7- (trifluoromethyl)isoindolin-2-yl)piperidine- 2,6-dione |
| 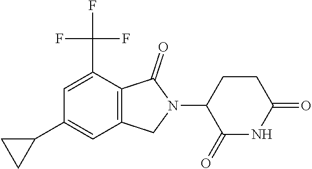 [SMILES: O=C1CCC(N2Cc3cc(C4CC4)cc(C(F)(F)F)c3C2=O)C(=O)N1] | 3-(5-cyclopropyl-1-oxo-7- (trifluoromethyl)isoindolin-2-yl)piperidine- 2,6-dione |
| 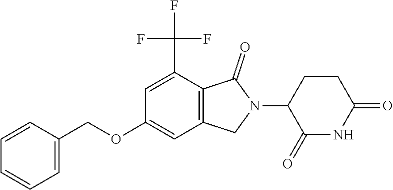 [SMILES: O=C1CCC(N2Cc3cc(OCc4ccccc4)cc(C(F)(F)F)c3C2=O)C(=O)N1] | 3-(5-(benzyloxy)-1-oxo-7- (trifluoromethyl)isoindolin-2-yl)piperidine- 2,6-dione |
| 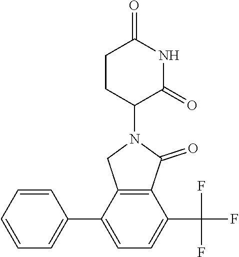 [SMILES: O=C1CCC(N2Cc3c(-c4ccccc4)ccc(C(F)(F)F)c3C2=O)C(=O)N1] | 3-(1-oxo-4-phenyl-7- (trifluoromethyl)isoindolin-2-yl)piperidine- 2,6-dione |
| 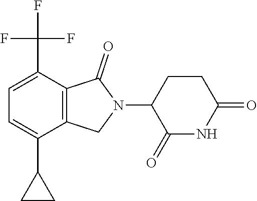 [SMILES: O=C1CCC(N2Cc3c(C4CC4)ccc(C(F)(F)F)c3C2=O)C(=O)N1] | 3-(4-cyclopropyl-1-oxo-7- (trifluoromethyl)isoindolin-2-yl)piperidine- 2,6-dione |
| 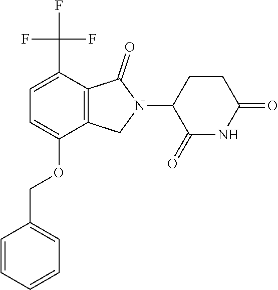 [SMILES: O=C1CCC(N2Cc3c(OCc4ccccc4)ccc(C(F)(F)F)c3C2=O)C(=O)N1] | 3-(4-(benzyloxy)-1-oxo-7- (trifluoromethyl)isoindolin-2-yl)piperidine- 2,6-dione |
| 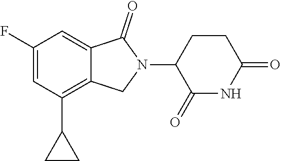 [SMILES: O=C1CCC(N2Cc3c(cc(F)cc3C3CC3)C2=O)C(=O)N1] | 3-(4-cyclopropyl-6-fluoro-1-oxoisoindolin- 2-yl)piperidine-2,6-dione |
| 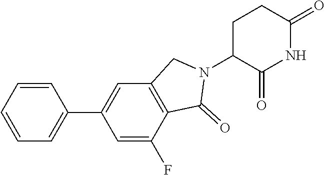 [SMILES: O=C1CCC(N2Cc3cc(-c4ccccc4)cc(F)c3C2=O)C(=O)N1] | 3-(7-fluoro-1-oxo-5-phenylisoindolin-2- yl)piperidine-2,6-dione |
| 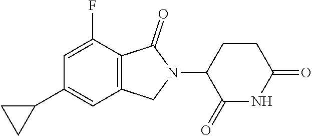 [SMILES: O=C1CCC(N2Cc3cc(C4CC4)cc(F)c3C2=O)C(=O)N1] | 3-(5-cyclopropyl-7-fluoro-1-oxoisoindolin- 2-yl)piperidine-2,6-dione |
| 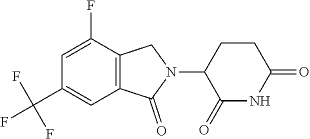 [SMILES: O=C1CCC(N2Cc3c(F)cc(C(F)(F)F)cc3C2=O)C(=O)N1] | 3-(4-fluoro-1-oxo-6- (trifluoromethyl)isoindolin-2-yl)piperidine- 2,6-dione |
| 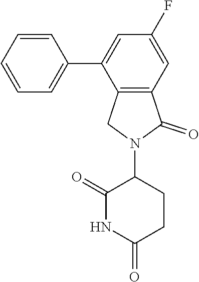 [SMILES: O=C1CCC(N2Cc3c(cc(F)cc3-c3ccccc3)C2=O)C(=O)N1] | 3-(6-fluoro-1-oxo-4-phenylisoindolin-2- yl)piperidine-2,6-dione |
| 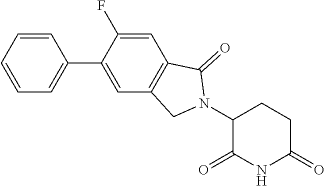 [SMILES: O=C1CCC(N2Cc3cc(-c4ccccc4)c(F)cc3C2=O)C(=O)N1] | 3-(6-fluoro-1-oxo-5-phenylisoindolin-2- yl)piperidine-2,6-dione |
| 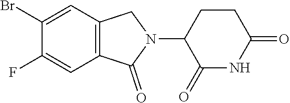 [SMILES: O=C1CCC(N2Cc3cc(Br)c(F)cc3C2=O)C(=O)N1] | 3-(5-bromo-6-fluoro-1-oxoisoindolin-2- yl)piperidine-2,6-dione |
| 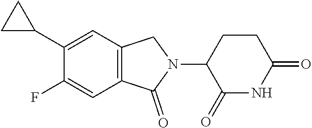 [SMILES: O=C1CCC(N2Cc3cc(C4CC4)c(F)cc3C2=O)C(=O)N1] | 3-(5-cyclopropyl-6-fluoro-1-oxoisoindolin- 2-yl)piperidine-2,6-dione |
| 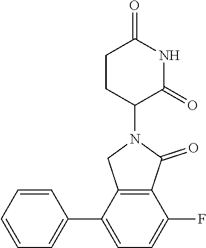 [SMILES: O=C1CCC(N2Cc3c(-c4ccccc4)ccc(F)c3C2=O)C(=O)N1] | 3-(7-fluoro-1-oxo-4-phenylisoindolin-2- yl)piperidine-2,6-dione |
| 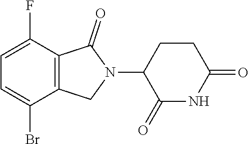 [SMILES: O=C1CCC(N2Cc3c(Br)ccc(F)c3C2=O)C(=O)N1] | 3-(4-bromo-7-fluoro-1-oxoisoindolin-2- yl)piperidine-2,6-dione |
| 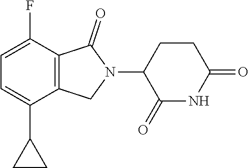 [SMILES: O=C1CCC(N2Cc3c(C4CC4)ccc(F)c3C2=O)C(=O)N1] | 3-(4-cyclopropyl-7-fluoro-1-oxoisoindolin- yl)piperidine-2,6-dione |
| 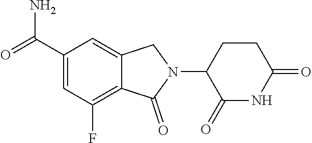 [SMILES: NC(=O)c1cc(F)c2c(c1)CN(C1CCC(=O)NC1=O)C2=O] | 2-(2,6-dioxopiperidin-3-yl)-7-fluoro-1- oxoisoindoline-5-carboxamide |
| 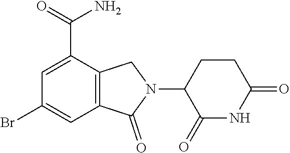 [SMILES: NC(=O)c1cc(Br)cc2c1CN(C1CCC(=O)NC1=O)C2=O] | 6-bromo-2-(2,6-dioxopiperidin-3-yl)-1- oxoisoindoline-4-carboxamide |
| 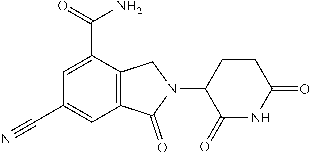 [SMILES: N#Cc1cc(C(N)=O)c2c(c1)C(=O)N(C1CCC(=O)NC1=O)C2] | 6-cyano-2-(2,6-dioxopiperidin-3-yl)-1- oxoisoindoline-4-carboxamide |
| 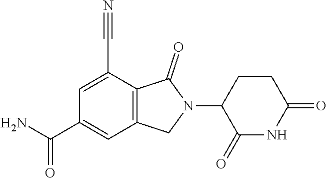 [SMILES: N#Cc1cc(C(N)=O)cc2c1C(=O)N(C1CCC(=O)NC1=O)C2] | 7-cyano-2-(2,6-dioxopiperidin-3-yl)-1- oxoisoindoline-5-carboxamide |
| 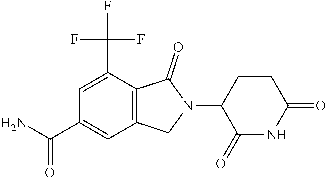 [SMILES: NC(=O)c1cc2c(c(C(F)(F)F)c1)C(=O)N(C1CCC(=O)NC1=O)C2] | 2-(2,6-dioxopiperidin-3-yl)-1-oxo-7- (trifluoromethyl)isoindoline-5-carboxamide |
| 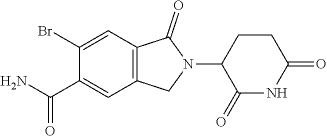 [SMILES: NC(=O)c1cc2c(cc1Br)C(=O)N(C1CCC(=O)NC1=O)C2] | 6-bromo-2-(2,6-dioxopiperidin-3-yl)-1- oxoisoindoline-5-carboxamide |
| 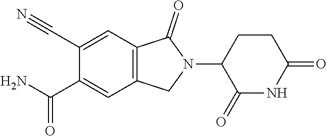 [SMILES: N#Cc1cc2c(cc1C(N)=O)CN(C1CCC(=O)NC1=O)C2=O] | 6-cyano-2-(2,6-dioxopiperidin-3-yl)-1- oxoisoindoline-5-carboxamide |
| 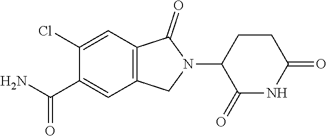 [SMILES: NC(=O)c1cc2c(cc1Cl)C(=O)N(C1CCC(=O)NC1=O)C2] | 6-chloro-2-(2,6-dioxopiperidin-3-yl)-1- oxoisoindoline-5-carboxamide |
| 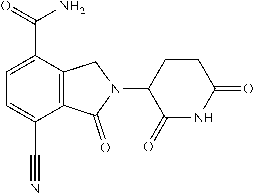 [SMILES: N#Cc1ccc(C(N)=O)c2c1C(=O)N(C1CCC(=O)NC1=O)C2] | 7-cyano-2-(2,6-dioxopiperidin-3-yl)-1- oxoisoindoline-4-carboxamide |
| 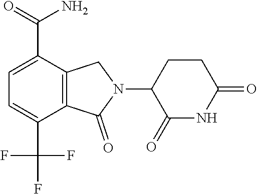 [SMILES: NC(=O)c1ccc(C(F)(F)F)c2c1CN(C1CCC(=O)NC1=O)C2=O] | 2-(2,6-dioxopiperidin-3-yl)-1-oxo-7- (trifluoromethyl)isoindoline-4-carboxamide |
| 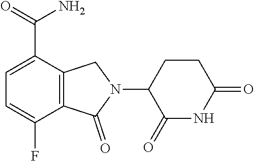 [SMILES: NC(=O)c1ccc(F)c2c1CN(C1CCC(=O)NC1=O)C2=O] | 2-(2,6-dioxopiperidin-3-yl)-7-fluoro-1- oxoisoindoline-4-carboxamide |
| 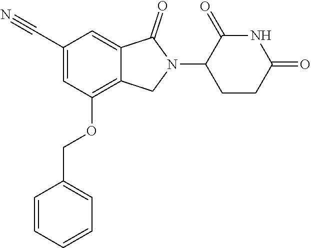 [SMILES: N#Cc1cc(OCc2ccccc2)c2c(c1)C(=O)N(C1CCC(=O)NC1=O)C2] | 7-(benzyloxy)-2-(2,6-dioxopiperidin-3-yl)- 3-oxoisoindoline-5-carbonitrile |
| 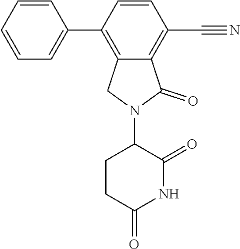 [SMILES: N#Cc1ccc(-c2ccccc2)c2c1C(=O)N(C1CCC(=O)NC1=O)C2] | 2-(2,6-dioxopiperidin-3-yl)-3-oxo-7- phenylisoindoline-4-carbonitrile |
| 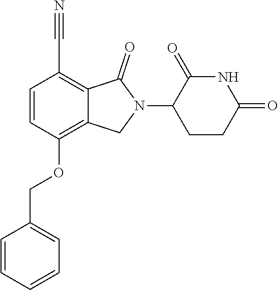 [SMILES: N#Cc1ccc(OCc2ccccc2)c2c1C(=O)N(C1CCC(=O)NC1=O)C2] | 7-(benzyloxy)-2-(2,6-dioxopiperidin-3-yl)- 3-oxoisoindoline-4-carbonitrile |
| 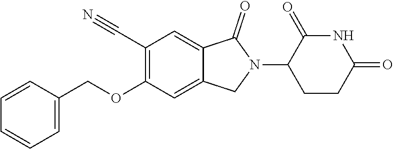 [SMILES: N#Cc1cc2c(cc1OCc1ccccc1)CN(C1CCC(=O)NC1=O)C2=O] | 6-(benzyloxy)-2-(2,6-dioxopiperidin-3-yl)- 3-oxoisoindoline-5-carbonitrile |
| 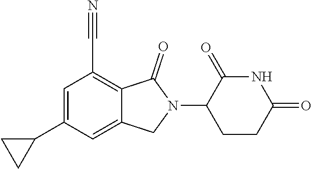 [SMILES: N#Cc1cc(C2CC2)cc2c1C(=O)N(C1CCC(=O)NC1=O)C2] | 6-cyclopropyl-2-(2,6-dioxopiperidin-3-yl)- 3-oxoisoindoline-4-carbonitrile |
| 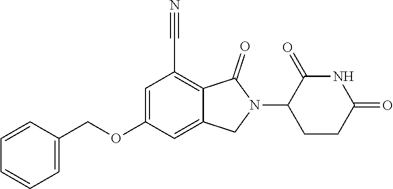 [SMILES: N#Cc1cc(OCc2ccccc2)cc2c1C(=O)N(C1CCC(=O)NC1=O)C2] | 6-(benzyloxy)-2-(2,6-dioxopiperidin-3-yl)- 3-oxoisoindoline-4-carbonitrile |
| 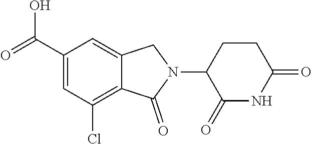 [SMILES: O=C1CCC(N2Cc3cc(C(=O)O)cc(Cl)c3C2=O)C(=O)N1] | 7-chloro-2-(2,6-dioxopiperidin-3-yl)-1- oxoisoindoline-5-carboxylic acid |
| 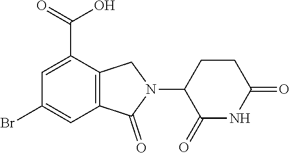 [SMILES: O=C1CCC(N2Cc3c(C(=O)O)cc(Br)cc3C2=O)C(=O)N1] | 6-bromo-2-(2,6-dioxopiperidin-3-yl)-1- oxoisoindoline-4-carboxylic acid |
| 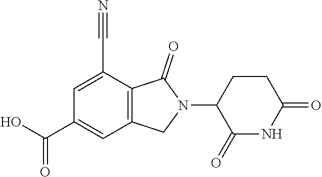 [SMILES: N#Cc1cc(C(=O)O)cc2c1C(=O)N(C1CCC(=O)NC1=O)C2] | 7-cyano-2-(2,6-dioxopiperidin-3-yl)-1- oxoisoindoline-5-carboxylic acid |
| 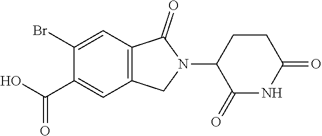 [SMILES: O=C1CCC(N2Cc3cc(C(=O)O)c(Br)cc3C2=O)C(=O)N1] | 6-bromo-2-(2,6-dioxopiperidin-3-yl)-1- oxoisoindoline-5-carboxylic acid |
| 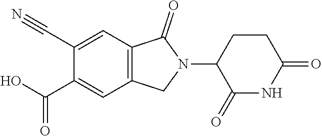 [SMILES: N#Cc1cc2c(cc1C(=O)O)CN(C1CCC(=O)NC1=O)C2=O] | 6-cyano-2-(2,6-dioxopiperidin-3-yl)-1- oxoisoindoline-5-carboxylic acid |
| 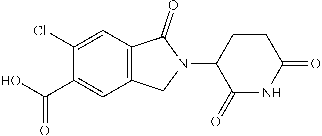 [SMILES: O=C1CCC(N2Cc3cc(C(=O)O)c(Cl)cc3C2=O)C(=O)N1] | 6-chloro-2-(2,6-dioxopiperidin-3-yl)-1- oxoisoindoline-5-carboxylic acid |
| 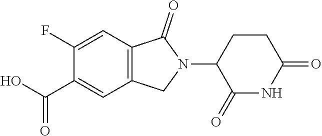 [SMILES: O=C1CCC(N2Cc3cc(C(=O)O)c(F)cc3C2=O)C(=O)N1] | 2-(2,6-dioxopiperidin-3-yl)-6-fluoro-1- oxoisoindoline-5-carboxylic acid |

TABLE 3

| Chemical Structure | Name |
|---|---|
| 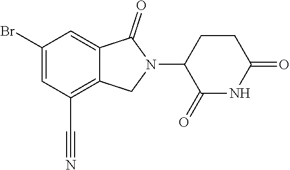 [SMILES: N#Cc1cc(Br)cc2c1CN(C1CCC(=O)NC1=O)C2=O] | 6-bromo-2-(2,6-dioxopiperidin-3-yl)-1- oxoisoindoline-4-carbonitrile |
| 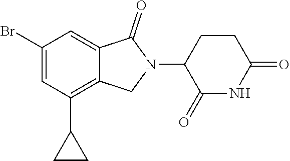 [SMILES: O=C1CCC(N2Cc3c(cc(Br)cc3C3CC3)C2=O)C(=O)N1] | 3-(6-bromo-4-cyclopropyl-1-oxoisoindolin- 2-yl)piperidine-2,6-dione |
| 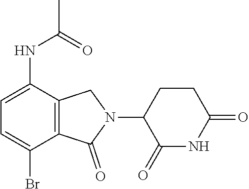 [SMILES: CC(=O)Nc1ccc(Br)c2c1CN(C1CCC(=O)NC1=O)C2=O] | N-(7-brommo-2-(2,6-dioxopiperidin-3-yl)-1- oxoisoindolin-4-yl)acetamide |
| 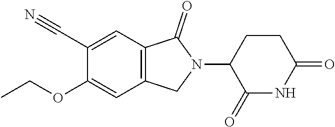 [SMILES: CCOc1cc2c(cc1C#N)C(=O)N(C1CCC(=O)NC1=O)C2] | 2-(2,6-dioxopiperidine-3-yl)-6-ethoxy-3- oxoisoindoline-5-carbonitrile |
| 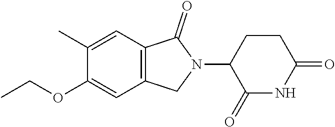 [SMILES: CCOc1cc2c(cc1C)C(=O)N(C1CCC(=O)NC1=O)C2] | 3-(5-ethoxy-6-methyl-1-oxoisoindolin-2- yl)piperidine-2,6-dione |
| 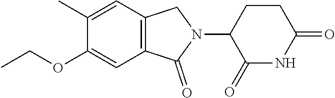 [SMILES: CCOc1cc2c(cc1C)CN(C1CCC(=O)NC1=O)C2=O] | 3-(6-ethoxy-5-methyl-1-oxoisoindolin-2- yl)piperidine-2,6-dione |
| 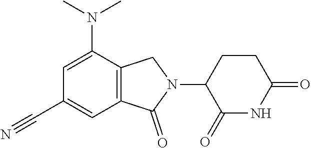 [SMILES: CN(C)c1cc(C#N)cc2c1CN(C1CCC(=O)NC1=O)C2=O] | 7-(dimethylamino)-2-(2,6-dioxopiperidin-3- yl)-3-oxoisoindoline-5-carbonitrile |
| 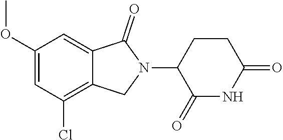 [SMILES: COc1cc(Cl)c2c(c1)C(=O)N(C1CCC(=O)NC1=O)C2] | 3-(4-chloro-6-methoxy-1-oxoisoindolin-2- yl)piperidine-2,6-dione |
| 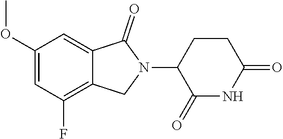 [SMILES: COc1cc(F)c2c(c1)C(=O)N(C1CCC(=O)NC1=O)C2] | 3-(4-fluoro-6-methoxy-1-oxoisoindolin-2- yl)piperidine-2,6-dione |
| 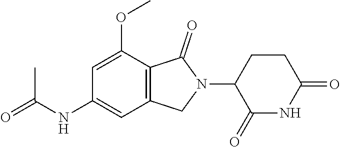 [SMILES: COc1cc(NC(C)=O)cc2c1C(=O)N(C1CCC(=O)NC1=O)C2] | N-(2-(2,6-dioxopiperidin-3-yl)-7-methoxy-1- oxoisoindolin-5-yl)acetamide |
| 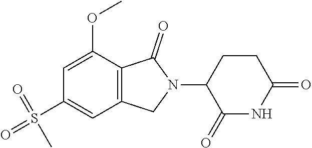 [SMILES: COc1cc(S(C)(=O)=O)cc2c1C(=O)N(C1CCC(=O)NC1=O)C2] | 3-(7-methoxy-5-(methylsulfonyl)-1- oxoisoindolin-2-yl)piperidine-2,6-dione |
| 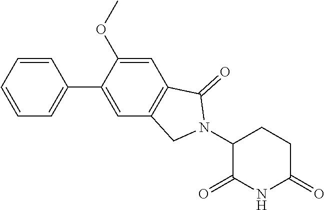 [SMILES: COc1cc2c(cc1-c1ccccc1)CN(C1CCC(=O)NC1=O)C2=O] | 3-(6-methoxy-1-oxo-5-phenylisoindolin-2- yl)piperidine-2,6-dione |
| 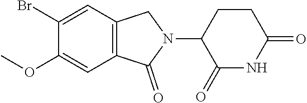 [SMILES: COc1cc2c(cc1Br)CN(C1CCC(=O)NC1=O)C2=O] | 3-(5-bromo-6-methoxy-1-oxoisoindolin-2- yl)piperidine-2,6-dione |
| 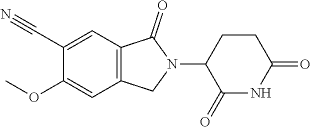 [SMILES: COc1cc2c(cc1C#N)C(=O)N(C1CCC(=O)NC1=O)C2] | 2-(2,6-dioxopiperidin-3-yl)-6-methoxy-3- oxoisoindoline-5-carbonitrile |
| 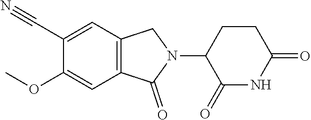 [SMILES: COc1cc2c(cc1C#N)CN(C1CCC(=O)NC1=O)C2=O] | 2-(2,6-dioxopiperidin-3-yl)-6-methoxy-1- oxoisoindoline-5-carbonitrile |
| 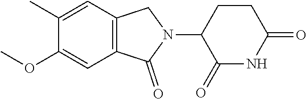 [SMILES: COc1cc2c(cc1C)CN(C1CCC(=O)NC1=O)C2=O] | 3-(6-methoxy-5-methyl-1-oxoisoindolin-2- yl)piperidine-2,6-dione |
| 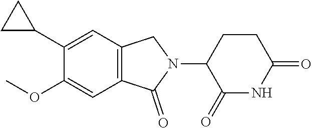 [SMILES: COc1cc2c(cc1C1CC1)CN(C1CCC(=O)NC1=O)C2=O] | 3-(5-cyclopropyl-6-methoxy-1- oxoisoindoline-2-yl)piperidine-2,6-dione |
| 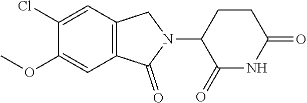 [SMILES: COc1cc2c(cc1Cl)CN(C1CCC(=O)NC1=O)C2=O] | 3-(5-chloro-6-methoxy-1-oxoisoindolin-2- yl)piperidine-2,6-dione |
| 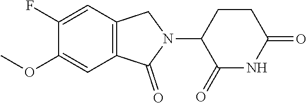 [SMILES: COc1cc2c(cc1F)CN(C1CCC(=O)NC1=O)C2=O] | 3-(5-fluoro-6-methoxy-1-oxoisoindolin-2- yl)piperidine-2,6-dione |
| 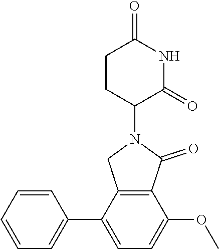 [SMILES: COc1ccc(-c2ccccc2)c2c1C(=O)N(C1CCC(=O)NC1=O)C2] | 3-(7-methoxy-1-oxo-4-phenylisoindolin-2- yl)piperidine-2,6-dione |
| 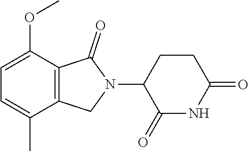 [SMILES: COc1ccc(C)c2c1C(=O)N(C1CCC(=O)NC1=O)C2] | 3-(7-methoxy-4-methyl-1-oxoisoindolin-2- yl)piperidine-2,6-dione |
| 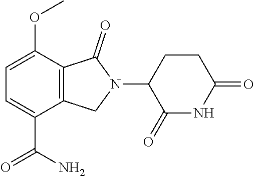 [SMILES: COc1ccc(C(N)=O)c2c1C(=O)N(C1CCC(=O)NC1=O)C2] | 2-(2,6-dioxopiperidin-3-yl)-7-methoxy-1- oxoisoindoline-4-carboxamide |
| 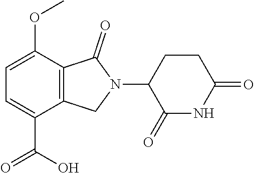 [SMILES: COc1ccc(C(=O)O)c2c1C(=O)N(C1CCC(=O)NC1=O)C2] | 2-(2,6-dioxopiperidin-3-yl)-7-methoxy-1- oxoisoindoline-4-carboxylic acid |
| 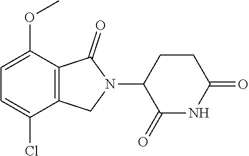 [SMILES: COc1ccc(Cl)c2c1C(=O)N(C1CCC(=O)NC1=O)C2] | 3-(4-chloro-7-methoxy-1-oxoisoindolin-2- yl)piperidine-2,6-dione |
| 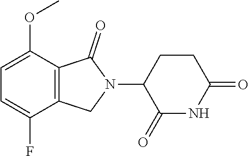 [SMILES: COc1ccc(F)c2c1C(=O)N(C1CCC(=O)NC1=O)C2] | 3-(4-fluoro-7-methoxy-1-oxoisoindolin-2- yl)piperidine-2,6-dione |
| 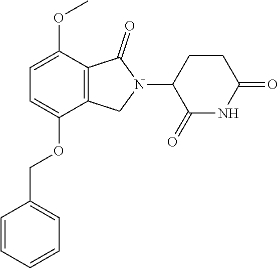 [SMILES: COc1ccc(OCc2ccccc2)c2c1C(=O)N(C1CCC(=O)NC1=O)C2] | 3-(4-(benzyloxy)-7-methoxy-1- oxoisoindolin-2-yl)piperidine-2,6-dione |
| 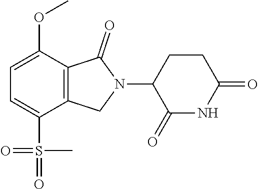 [SMILES: COc1ccc(S(C)(=O)=O)c2c1C(=O)N(C1CCC(=O)NC1=O)C2] | 3-(7-methoxy-4-(methylsulfonyl)-1- oxoisoindolin-2-yl)piperidine-2,6-dione |
| 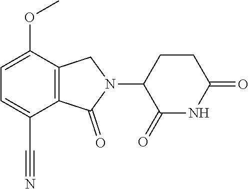 [SMILES: COc1ccc(C#N)c2c1CN(C1CCC(=O)NC1=O)C2=O] | 2-(2,6-dioxopiperidin-3-yl)-7-methoxy-3- oxoisoindoline-4-carbonitrile |
| 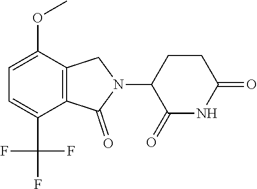 [SMILES: COc1ccc(C(F)(F)F)c2c1CN(C1CCC(=O)NC1=O)C2=O] | 3-(4-methoxy-1-oxo-7- (trifluoromethyl)isoindolin-2-yl)piperidine- 2,6-dione |
| 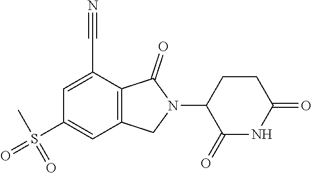 [SMILES: CS(=O)(=O)c1cc(C#N)c2c(c1)CN(C1CCC(=O)NC1=O)C2=O] | 2-(2,6-dioxopiperidin-3-yl)-6- (methylsulfonyl)-3-oxoisoindoline-4- carbonitrile |
| 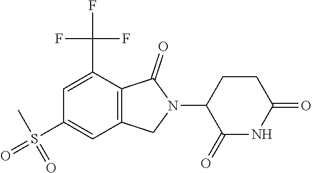 [SMILES: CS(=O)(=O)c1cc2c(c(C(F)(F)F)c1)C(=O)N(C1CCC(=O)NC1=O)C2] | 3-(5-(methylsulfonyl)-1-oxo-7- (trifluoromethyl)isoindolin-2-yl)piperidine- 2,6-dione |
| 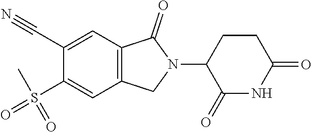 [SMILES: CS(=O)(=O)c1cc2c(cc1C#N)C(=O)N(C1CCC(=O)NC1=O)C2] | 2-(2,6-dioxopiperidin-3-yl)-6- (methylsulfonyl)-3-oxoisoindoline-5- carbonitrile |
| 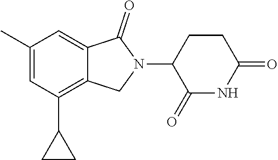 [SMILES: Cc1cc2c(c(C3CC3)c1)CN(C1CCC(=O)NC1=O)C2=O] | 3-(4-cyclopropyl-6-methyl-1-oxoisoindolin- 2-yl)piperidine-2,6-dione |
| 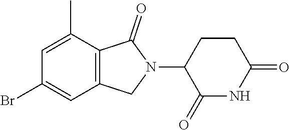 [SMILES: Cc1cc(Br)cc2c1C(=O)N(C1CCC(=O)NC1=O)C2] | 3-(5-bromo-7-methyl-1-oxoisoindolin-2- yl)piperidine-2,6-dione |
| 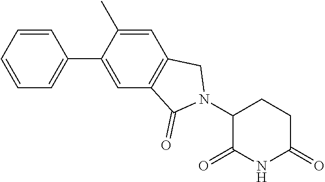 [SMILES: Cc1cc2c(cc1-c1ccccc1)C(=O)N(C1CCC(=O)NC1=O)C2] | 3-(5-methyl-1-oxo-6-phenylisoindolin-2- yl)piperidine-2,6-dione |
| 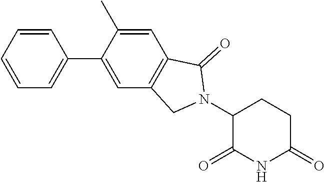 [SMILES: Cc1cc2c(cc1-c1ccccc1)CN(C1CCC(=O)NC1=O)C2=O] | 3-(6-methyl-1-oxo-5-phenylisoindolin-2- yl)piperidine-2,6-dione |
| 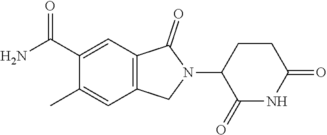 [SMILES: Cc1cc2c(cc1C(N)=O)C(=O)N(C1CCC(=O)NC1=O)C2] | 2-(2,6-dioxopiperidin-3-yl)-6-methyl-3- oxoisoindoline-5-carboxamide |
| 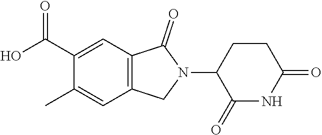 [SMILES: Cc1cc2c(cc1C(=O)O)C(=O)N(C1CCC(=O)NC1=O)C2] | 2-(2,6-dioxopiperidin-3-yl)-6-methyl-3- oxoisoindoline-5-carboxylic acid |
| 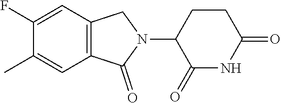 [SMILES: Cc1cc2c(cc1F)CN(C1CCC(=O)NC1=O)C2=O] | 3-(5-fluoro-6-methyl-1-oxoisoindolin-2- yl)piperidine-2,6-dione |
| 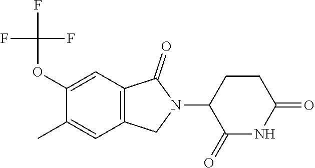 [SMILES: Cc1cc2c(cc1OC(F)(F)F)C(=O)N(C1CCC(=O)NC1=O)C2] | 3-(5-methyl-1-oxo-6- (trifluoromethoxy)isoindolin-2-yl)piperidine- 2,6-dione |
| 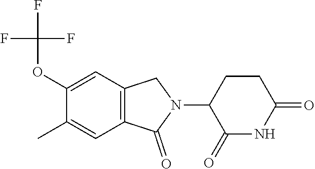 [SMILES: Cc1cc2c(cc1OC(F)(F)F)CN(C1CCC(=O)NC1=O)C2=O] | 3-(6-methyl-1-oxo-5- (trifluoromethoxy)isoindolin-2-yl)piperidine- 2,6-dione |
| 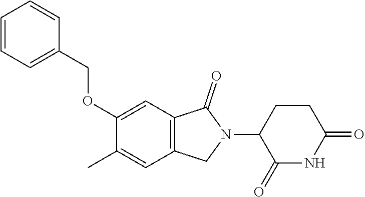 [SMILES: Cc1cc2c(cc1OCc1ccccc1)C(=O)N(C1CCC(=O)NC1=O)C2] | 3-(6-(benzyloxy)-5-methyl-1-oxoisoindolin- 2-yl)piperidine-2,6-dione |
| 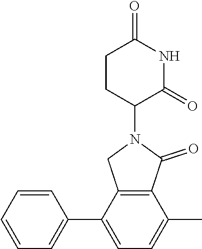 [SMILES: Cc1ccc(-c2ccccc2)c2c1C(=O)N(C1CCC(=O)NC1=O)C2] | 3-(7-methyl-1-oxo-4-phenylisoindolin-2- yl)piperidine-2,6-dione |
| 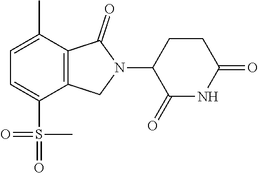 [SMILES: Cc1ccc(S(C)(=O)=O)c2c1C(=O)N(C1CCC(=O)NC1=O)C2] | 3-(7-methyl-4-(methylsulfonyl)-1- oxoisoindolin-2-yl)piperidine-2,6-dione |
| 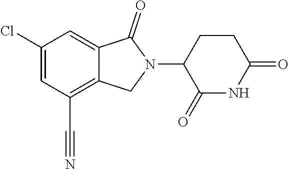 [SMILES: N#Cc1cc(Cl)cc2c1CN(C1CCC(=O)NC1=O)C2=O] | 6-chloro-2-(2,6-dioxopiperidin-3-yl)-1- oxoisoindoline-4-carbonitrile |
| 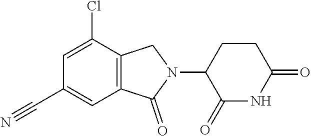 [SMILES: N#Cc1cc(Cl)c2c(c1)C(=O)N(C1CCC(=O)NC1=O)C2] | 7-chloro-2-(2,6-dioxopiperidin-3-yl)-3- oxoisoindoline-5-carbonitrile |
| 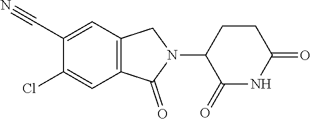 [SMILES: N#Cc1cc2c(cc1Cl)C(=O)N(C1CCC(=O)NC1=O)C2] | 6-chloro-2-(2,6-dioxopiperidin-3-yl)-1- oxoisoindoline-5-carbonitrile |
| 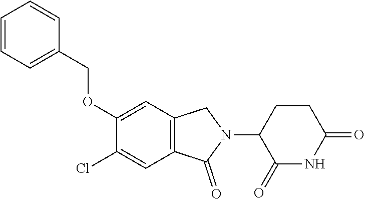 [SMILES: O=C1CCC(N2Cc3cc(OCc4ccccc4)c(Cl)cc3C2=O)C(=O)N1] | 3-(5-(benzyloxy)-6-chloro-1-oxoisoindolin- 2-yl)piperidine-2,6-dione |
| 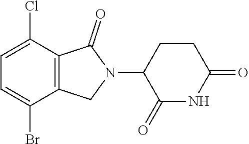 [SMILES: O=C1CCC(N2Cc3c(Br)ccc(Cl)c3C2=O)C(=O)N1] | 3-(4-bromo-7-chloro-1-oxoisoindolin-2- yl)piperidine-2,6-dione |
| 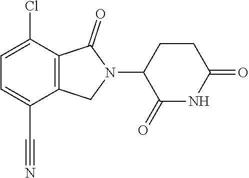 [SMILES: N#Cc1ccc(Cl)c2c1CN(C1CCC(=O)NC1=O)C2=O] | 7-chloro-2-(2,6-dioxopiperidin-3-yl)-1- oxoisoindoline-4-carbonitrile |
| 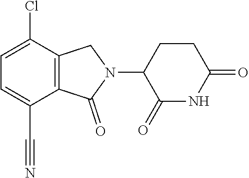 [SMILES: N#Cc1ccc(Cl)c2c1C(=O)N(C1CCC(=O)NC1=O)C2] | 7-chloro-2-(2,6-dioxopiperidin-3-yl)-3- oxoisoindoline-4-carbonitrile |
| 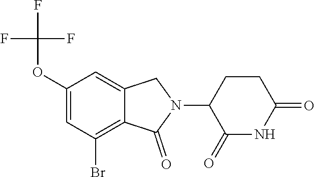 [SMILES: O=C1CCC(N2Cc3cc(OC(F)(F)F)cc(Br)c3C2=O)C(=O)N1] | 3-(7-bromo-1-oxo-5- (trifluoromethoxy)isoindolin-2-yl)piperidine- 2,6-dione |
| 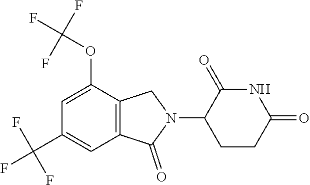 [SMILES: O=C1CCC(N2Cc3c(OC(F)(F)F)cc(C(F)(F)F)cc3C2=O)C(=O)N1] | 3-(1-oxo-4-(trifluoromethoxy)-6- (trifluoromethyl)isoindolin-2-yl)piperidine-2,6-dione |
| 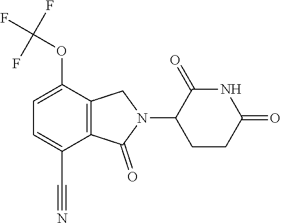 [SMILES: N#Cc1ccc(OC(F)(F)F)c2c1C(=O)N(C1CCC(=O)NC1=O)C2] | 2-(2,6-dioxopiperidin-3-yl)-3-oxo-7- (trifluoromethoxy)isoindoline-4-carbonitrile |
| 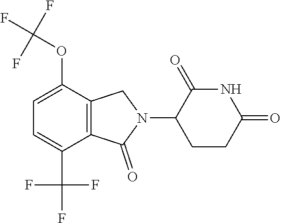 [SMILES: O=C1CCC(N2Cc3c(OC(F)(F)F)ccc(C(F)(F)F)c3C2=O)C(=O)N1] | 3-(1-oxo-4-(trifluoromethoxy)-7- (trifluoromethyl)isoindolin-2-yl)piperidine- 2,6-dione |
| 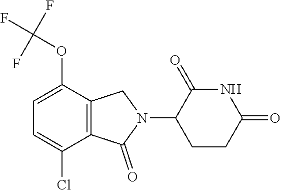 [SMILES: O=C1CCC(N2Cc3c(OC(F)(F)F)ccc(Cl)c3C2=O)C(=O)N1] | 3-(7-chloro-1-oxo-4- (trifluoromethoxy)isoindolin-2-yl)piperidine- 2,6-dione |
| 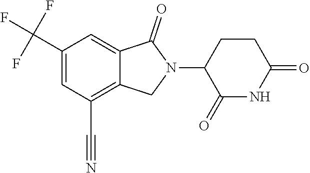 [SMILES: N#Cc1cc(C(F)(F)F)cc2c1CN(C1CCC(=O)NC1=O)C2=O] | 2-(2,6-dioxopiperidin-3-yl)-1-oxo-6- (trifluoromethyl)isoindoline-4-carbonitrile |
| 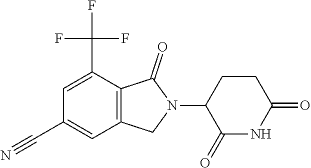 [SMILES: N#Cc1cc2c(c(C(F)(F)F)c1)C(=O)N(C1CCC(=O)NC1=O)C2] | 2-(2,6-dioxopiperidin-3-yl)-1-oxo-7- (trifluoromethyl)isoindoline-5-carbonitrile |
| 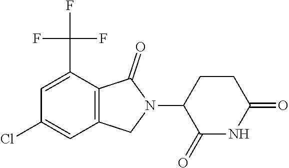 [SMILES: O=C1CCC(N2Cc3cc(Cl)cc(C(F)(F)F)c3C2=O)C(=O)N1] | 3-(5-chloro-1-oxo-7- (trifluoromethyl)isoindolin-2-yl)piperidine- 2,6-dione |
| 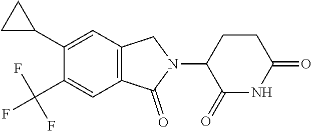 [SMILES: O=C1CCC(N2Cc3cc(C4CC4)c(C(F)(F)F)cc3C2=O)C(=O)N1] | 3-(5-cyclopropyl-1-oxo-6- (trifluoromethyl)isoindolin-2-yl)piperidine- 2,6-dione |
| 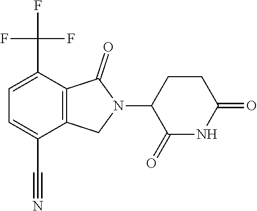 [SMILES: N#Cc1ccc(C(F)(F)F)c2c1CN(C1CCC(=O)NC1=O)C2=O] | 2-(2,6-dioxopiperidin-3-yl)-1-oxo-7- (trifluoromethyl)isoindoline-4-carbonitrile |
| 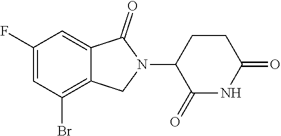 [SMILES: O=C1CCC(N2Cc3c(Br)cc(F)cc3C2=O)C(=O)N1] | 3-(4-bromo-6-fluoro-1-oxoisoindolin-2- yl)piperidine-2,6-dione |
| 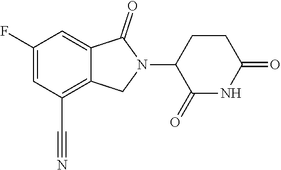 [SMILES: N#Cc1cc(F)cc2c1CN(C1CCC(=O)NC1=O)C2=O] | 2-(2,6-dioxopiperidin-3-yl)-6-fluoro-1- oxoisoindoline-4-carbonitrile |
| 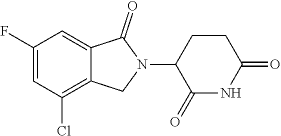 [SMILES: O=C1CCC(N2Cc3c(Cl)cc(F)cc3C2=O)C(=O)N1] | 3-(4-chloro-6-fluoro-1-oxoisoindolin-2- yl)piperidine-2,6-dione |
| 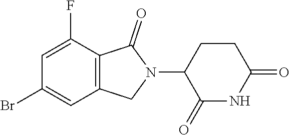 [SMILES: O=C1CCC(N2Cc3cc(Br)cc(F)c3C2=O)C(=O)N1] | 3-(5-bromo-7-fluoro-1-oxoisoindolin-2- yl)piperidine-2,6-dione |
| 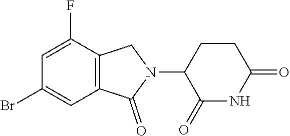 [SMILES: O=C1CCC(N2Cc3c(F)cc(Br)cc3C2=O)C(=O)N1] | 3-(6-bromo-4-fluoro-1-oxoisoindolin-2- yl)piperidine-2,6-dione |
| 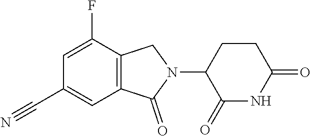 [SMILES: N#Cc1cc(F)c2c(c1)C(=O)N(C1CCC(=O)NC1=O)C2] | 2-(2,6-dioxopiperidin-3-yl)-7-fluoro-3- oxoisoindoline-5-carbonitrile |
| 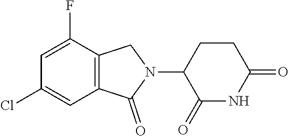 [SMILES: O=C1CCC(N2Cc3c(F)cc(Cl)cc3C2=O)C(=O)N1] | 3-(6-chloro-4-fluoro-1-oxoisoindolin-2- yl)piperidine-2,6-dione |
| 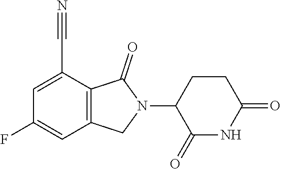 [SMILES: N#Cc1cc(F)cc2c1C(=O)N(C1CCC(=O)NC1=O)C2] | 2-(2,6-dioxopiperidin-3-yl)-6-fluoro-3- oxoisoindoline-4-carbonitrile |
| 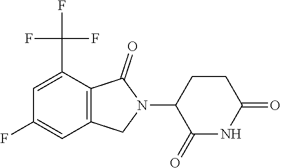 [SMILES: O=C1CCC(N2Cc3cc(F)cc(C(F)(F)F)c3C2=O)C(=O)N1] | 3-(5-fluoro-1-oxo-7- (trifluoromethyl)isoindolin-2-yl)piperidine- 2,6-dione |
| 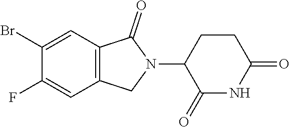 [SMILES: O=C1CCC(N2Cc3cc(F)c(Br)cc3C2=O)C(=O)N1] | 3-(6-bromo-5-fluoro-1-oxoisoindolin-2- yl)piperidine-2,6-dione |
| 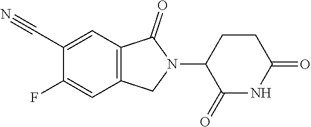 [SMILES: N#Cc1cc2c(cc1F)CN(C1CCC(=O)NC1=O)C2=O] | 2-(2,6-dioxopiperidin-3-yl)-6-fluoro-3- oxoisoindoline-5-carbonitrile |
| 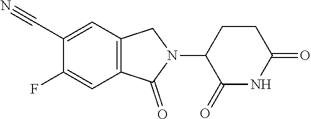 [SMILES: N#Cc1cc2c(cc1F)C(=O)N(C1CCC(=O)NC1=O)C2] | 2-(2,6-dioxopiperidin-3-yl)-6-fluoro-1- oxoisoindoline-5-carbonitrile |
| 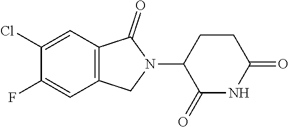 [SMILES: O=C1CCC(N2Cc3cc(F)c(Cl)cc3C2=O)C(=O)N1] | 3-(6-chloro-5-fluoro-1-oxoisoindolin-2-yl) piperidine-2,6-dione |
| 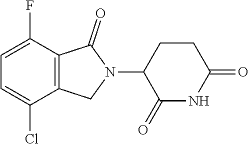 [SMILES: O=C1CCC(N2Cc3c(Cl)ccc(F)c3C2=O)C(=O)N1] | 3-(4-chloro-7-fluoro-1-oxoisoindolin-2- yl)piperidine-2,6-dione |
| 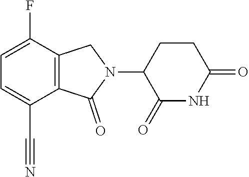 [SMILES: N#Cc1ccc(F)c2c1C(=O)N(C1CCC(=O)NC1=O)C2] | 2-(2,6-dioxopiperidin-3-yl)-7-fluoro-3- oxoisoindoline-4-carbonitrile |
| 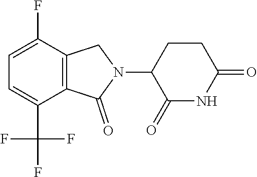 [SMILES: O=C1CCC(N2Cc3c(F)ccc(C(F)(F)F)c3C2=O)C(=O)N1] | 3-(4-fluoro-1-oxo-7- (trifluoromethyl)isoindolin-2-yl)piperidine- 2,6-dione |
| 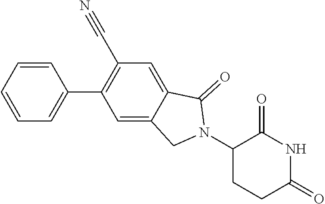 [SMILES: N#Cc1cc2c(cc1-c1ccccc1)CN(C1CCC(=O)NC1=O)C2=O] | 2-(2,6-dioxopiperidin-3-yl)-3-oxo-6- phenylisoindoline-5-carbonitrile |
| 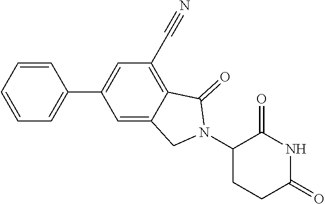 [SMILES: N#Cc1cc(-c2ccccc2)cc2c1C(=O)N(C1CCC(=O)NC1=O)C2] | 2-(2,6-dioxopiperidin-3-yl)-3-oxo-6- phenylisoindoline-4-carbonitrile |

TABLE 4

| Chemical Structure | Name |
|---|---|
| 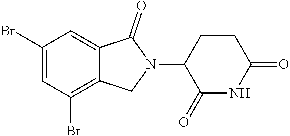 [SMILES: O=C1CCC(N2Cc3c(Br)cc(Br)cc3C2=O)C(=O)N1] | 3-(4,6-dibromo-1- oxoisoindolin-2-yl)piperidine- 2,6-dione |
| 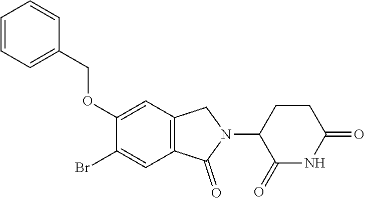 [SMILES: O=C1CCC(N2Cc3cc(OCc4ccccc4)c(Br)cc3C2=O)C(=O)N1] | 3-(5-(benzyloxy)-6-bromo-1- oxoisoindolin-2-yl)piperidine- 2,6-dione |
| 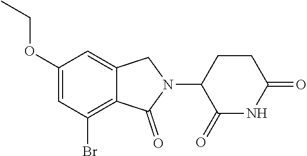 [SMILES: CCOc1cc(Br)c2c(c1)CN(C1CCC(=O)NC1=O)C2=O] | 3-(7-bromo-5-ethoxy-1- oxoisoindolin-2-y)piperidine- 2,6-dione |
| 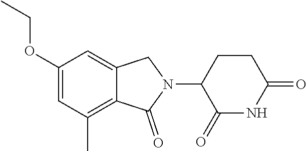 [SMILES: CCOc1cc(C)c2c(c1)CN(C1CCC(=O)NC1=O)C2=O] | 3-(5-ethoxy-7-methyl-1- oxoisoindolin-2-yl)piperidine- 2,6-dione |
| 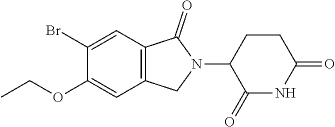 [SMILES: CCOc1cc2c(cc1Br)C(=O)N(C1CCC(=O)NC1=O)C2] | 3-(6-bromo-5-ethoxy-1- oxoisoindolin-2-yl)piperidine- 2,6-dione |
| 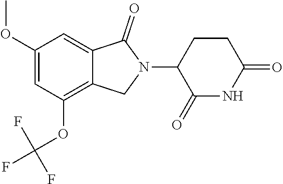 [SMILES: COc1cc(OC(F)(F)F)c2c(c1)C(=O)N(C1CCC(=O)NC1=O)C2] | 3-(6-methoxy-1-oxo-4- (trifluoromethoxy)isoindolin- 2-yl)piperidine-2,6-dione |
| 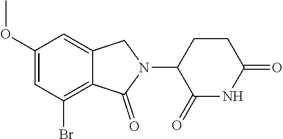 [SMILES: COc1cc(Br)c2c(c1)CN(C1CCC(=O)NC1=O)C2=O] | 3-(7-bromo-5-methoxy-1- oxoisoindolin-2-yl)piperidine- 2,6-dione |
| 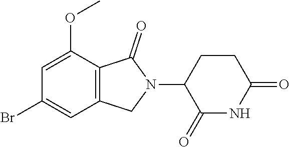 [SMILES: COc1cc(Br)cc2c1C(=O)N(C1CCC(=O)NC1=O)C2] | 3-(5-bromo-7-methoxy-1- oxoisoindolin-2-yl)piperidine- 2,6-dione |
| 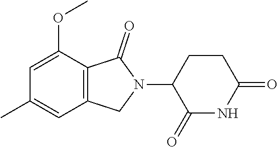 [SMILES: COc1cc(C)cc2c1C(=O)N(C1CCC(=O)NC1=O)C2] | 3-(7-methoxy-5-methyl-1- oxoisoindolin-2-yl)piperidine- 2,6-dione |
| 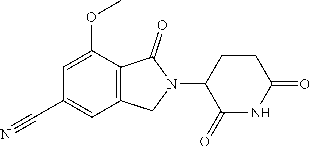 [SMILES: COc1cc(C#N)cc2c1C(=O)N(C1CCC(=O)NC1=O)C2] | 2-(2,6-dioxopiperidin-3-yl)-7- methoxy-1-oxoisoindoline-5- carbonitrile |
| 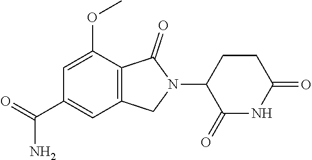 [SMILES: COc1cc(C(N)=O)cc2c1C(=O)N(C1CCC(=O)NC1=O)C2] | 2-(2,6-dioxopiperidin-3-yl)-7- methoxy-1-oxoisoindoline-5- carboxamide |
| 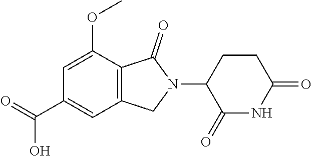 [SMILES: COc1cc(C(=O)O)cc2c1C(=O)N(C1CCC(=O)NC1=O)C2] | 2-(2,6-dioxopiperidin-3-yl)-7- methoxy-1-oxoisoindoline-5- carboxylic acid |
| 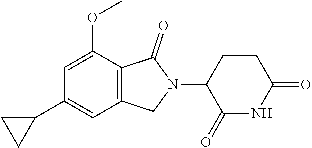 [SMILES: COc1cc(C2CC2)cc2c1C(=O)N(C1CCC(=O)NC1=O)C2] | 3-(5-cyclopropyl-7-methoxy-1- oxoisoindolin-2-yl)piperidine- 2,6-dione |
| 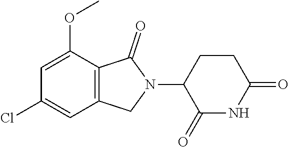 [SMILES: COc1cc(Cl)cc2c1C(=O)N(C1CCC(=O)NC1=O)C2] | 3-(5-chloro-7-methoxy-1- oxoisoindolin-2-yl)piperidine- 2,6-dione |
| 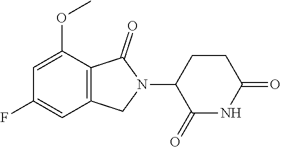 [SMILES: COc1cc(F)cc2c1C(=O)N(C1CCC(=O)NC1=O)C2] | 3-(5-fluoro-7-methoxy-1- oxoisoindolin-3-yl)piperidine- 2,6-dione |
| 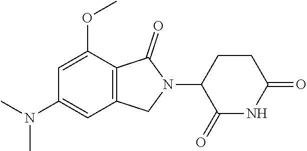 [SMILES: COc1cc(N(C)C)cc2c1C(=O)N(C1CCC(=O)NC1=O)C2] | 3-(5-(dimethylamino)-7- methoxy-1-oxoisoindolin-2- yl)piperidine-2,6-dione |
| 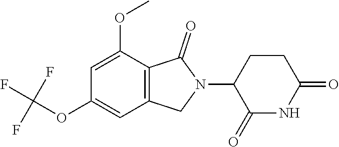 [SMILES: COc1cc(OC(F)(F)F)cc2c1C(=O)N(C1CCC(=O)NC1=O)C2] | 3-(7-methoxy-1-oxo-5- (trifluoromethoxy)isoindolin- 2-yl)piperidine-2,6-dione |
| 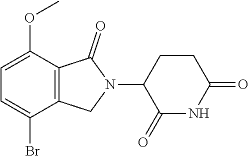 [SMILES: COc1ccc(Br)c2c1C(=O)N(C1CCC(=O)NC1=O)C2] | 3-(4-bromo-7-methoxy-1- oxoisoindolin-2-yl)piperidine- 2,6-dione |
| 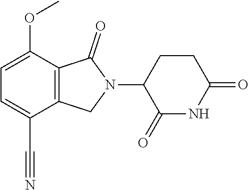 [SMILES: COc1ccc(C#N)c2c1C(=O)N(C1CCC(=O)NC1=O)C2] | 2-(2,6-dioxopiperidin-3-yl)-7- methoxy-1-oxoisoindoline-4- carbonitrile |
| 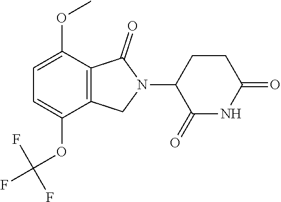 [SMILES: COc1ccc(OC(F)(F)F)c2c1C(=O)N(C1CCC(=O)NC1=O)C2] | 3-(7-methoxy-1-oxo-4- (trifluoromethoxy)isoindolin- 2-yl)piperidine-2,6-dione |
| 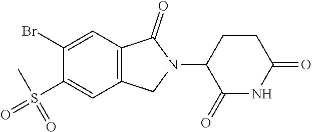 [SMILES: CS(=O)(=O)c1cc2c(cc1Br)C(=O)N(C1CCC(=O)NC1=O)C2] | 3-(6-bromo-5- (methylsulfonyl)-1- oxoisoindolin-2-yl)piperidine- 2,6-dione |
| 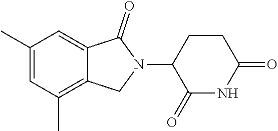 [SMILES: Cc1cc(C)c2c(c1)C(=O)N(C1CCC(=O)NC1=O)C2] | 3-(4,6-dimethyl-1- oxoisoindolin-2-yl)piperidine- 2,6-dione |
| 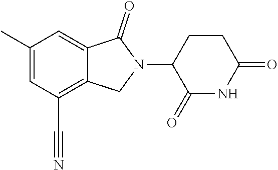 [SMILES: Cc1cc(C#N)c2c(c1)C(=O)N(C1CCC(=O)NC1=O)C2] | 2-(2,6-dioxopiperidin-3-yl)-6- methyl-1-oxoisoindoline-4- carbonitrile |
| 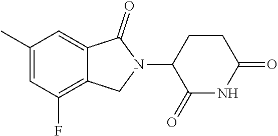 [SMILES: Cc1cc(F)c2c(c1)C(=O)N(C1CCC(=O)NC1=O)C2] | 3-(4-fluoro-6-methyl-1- oxoisoindolin-2-yl)piperidine- 2,6-dione |
| 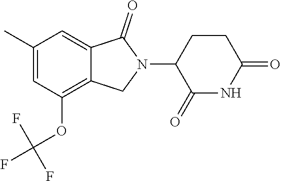 [SMILES: Cc1cc(OC(F)(F)F)c2c(c1)C(=O)N(C1CCC(=O)NC1=O)C2] | 3-(6-methyl-1-oxo-4- (trifluoromethoxy)isoindolin- 2-yl)piperidine-2,6-dione |
| 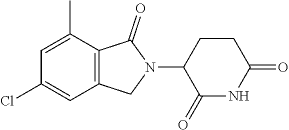 [SMILES: Cc1cc(Cl)cc2c1C(=O)N(C1CCC(=O)NC1=O)C2] | 3-(5-chloro-7-methyl-1- oxoisoindolin-2-yl)piperidine- 2,6-dione |
| 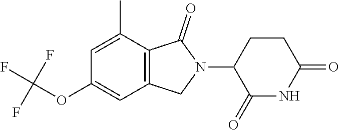 [SMILES: Cc1cc(OC(F)(F)F)cc2c1C(=O)N(C1CCC(=O)NC1=O)C2] | 3-(7-methyl-1-oxo-5- (trifluoromethoxy)isoindolin- 2-yl)piperidine-2,6-dione |
| 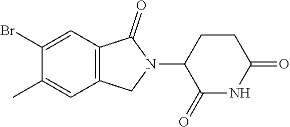 [SMILES: Cc1cc2c(cc1Br)C(=O)N(C1CCC(=O)NC1=O)C2] | 3-(6-bromo-5-methyl-1- oxoisoindolin-2-yl)piperidine- 2,6-dione |
| 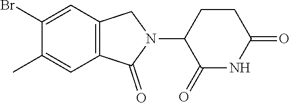 [SMILES: Cc1cc2c(cc1Br)CN(C1CCC(=O)NC1=O)C2=O] | 3-(5-bromo-6-methyl-1- oxoisoindolin-2-yl)piperidine- 2,6-dione |
| 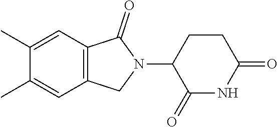 [SMILES: Cc1cc2c(cc1C)C(=O)N(C1CCC(=O)NC1=O)C2] | 3-(5,6-dimethyl-1- oxoisoindolin-2-yl)piperidine- 2,6-dione |
| 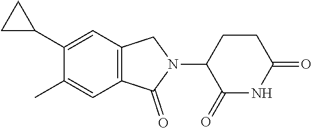 [SMILES: Cc1cc2c(cc1C1CC1)CN(C1CCC(=O)NC1=O)C2=O] | 3-(5-cyclopropyl-6-methyl-1- oxoisoindolin-2-yl)piperidine- 2,6-dione |
| 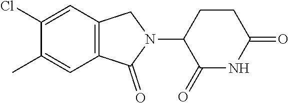 [SMILES: Cc1cc2c(cc1Cl)CN(C1CCC(=O)NC1=O)C2=O] | 3-(5-chloro-6-methyl-1- oxoisoindolin-2-yl)piperidine- 2,6-dione |
| 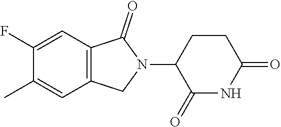 [SMILES: Cc1cc2c(cc1F)C(=O)N(C1CCC(=O)NC1=O)C2] | 3-(6-fluoro-5-methyl-1- oxoisoindolin-2-yl)piperidine- 2,6-dione |
| 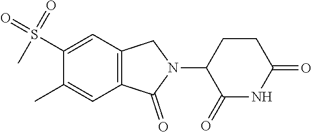 [SMILES: Cc1cc2c(cc1S(C)(=O)=O)CN(C1CCC(=O)NC1=O)C2=O] | 3-(6-methyl-5- (methylsulfonyl)-1- oxoisoindolin-2-yl)piperidine- 2,6-dione |
| 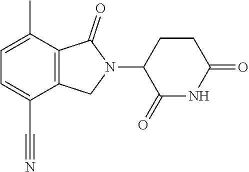 [SMILES: Cc1ccc(C#N)c2c1C(=O)N(C1CCC(=O)NC1=O)C2] | 2-(2,6-dioxopiperidin-3-yl)-7- methyl-1-oxoisoindoline-4- carbonitrile |
| 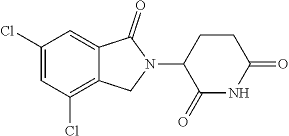 [SMILES: O=C1CCC(N2Cc3c(Cl)cc(Cl)cc3C2=O)C(=O)N1] | 3-(4,6-dichloro-1- oxoisoindolin-2-yl)piperidine- 2,6-dione |
| 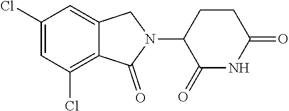 [SMILES: O=C1CCC(N2Cc3cc(Cl)cc(Cl)c3C2=O)C(=O)N1] | 3-(5,7-dichloro-1- oxoisoindolin-2-yl)piperidine- 2,6-dione |
| 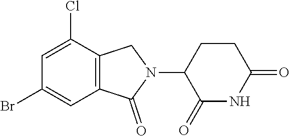 [SMILES: O=C1CCC(N2Cc3c(Cl)cc(Br)cc3C2=O)C(=O)N1] | 3-(6-bromo-4-chloro-1- oxoisoindolin-2-yl)piperidine- 2,6-dione |
| 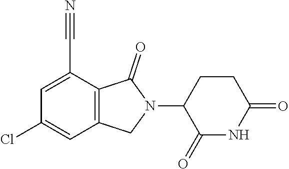 [SMILES: N#Cc1cc(Cl)cc2c1C(=O)N(C1CCC(=O)NC1=O)C2] | 6-chloro-2-(2,6- dioxopiperidin-3-yl)-3- oxoisoindoline-4-carbonitrile |
| 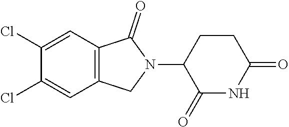 [SMILES: O=C1CCC(N2Cc3cc(Cl)c(Cl)cc3C2=O)C(=O)N1] | 3-(5,6-dichloro-1- oxoisoindolin-2-yl)piperidine- 2,6-dione |
| 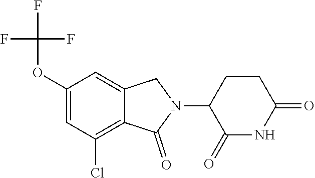 [SMILES: O=C1CCC(N2Cc3cc(OC(F)(F)F)cc(Cl)c3C2=O)C(=O)N1] | 3-(7-chloro-1-oxo-5- (trifluoromethoxy)isoindolin- 2-yl)piperidine-2,6-dione |
| 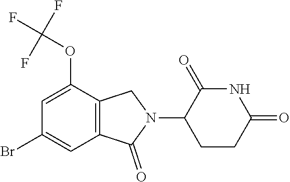 [SMILES: O=C1CCC(N2Cc3c(OC(F)(F)F)cc(Br)cc3C2=O)C(=O)N1] | 3-(6-bromo-1-oxo-4- (trifluoromethoxy)isoindolin- 2-yl)piperidine-2,6-dione |
| 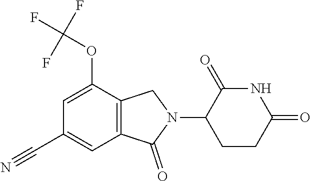 [SMILES: N#Cc1cc(OC(F)(F)F)c2c(c1)C(=O)N(C1CCC(=O)NC1=O)C2] | 2-(2,6-dioxopiperidin-3-yl)-3- oxo-7- (trifluoromethoxy)isoindoline- 5-carbonitrile |
| 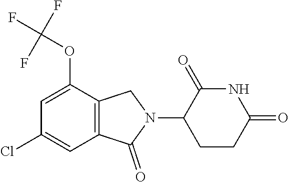 [SMILES: O=C1CCC(N2Cc3c(OC(F)(F)F)cc(Cl)cc3C2=O)C(=O)N1] | 3-(6-chloro-1-oxo-4- (trifluoromethoxy)isoindolin- 2-yl)piperidine-2,6-dione |
| 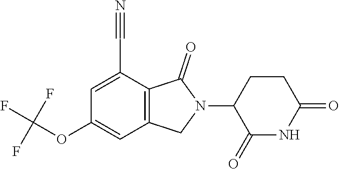 [SMILES: N#Cc1cc(OC(F)(F)F)cc2c1C(=O)N(C1CCC(=O)NC1=O)C2] | 2-(2,6-dioxopiperidin-3-yl)-3- oxo-6- (trifluoromethoxy)isoindoline- 4-carbonitrile |
| 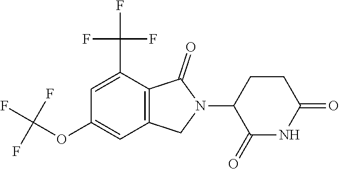 [SMILES: O=C1CCC(N2Cc3cc(OC(F)(F)F)cc(C(F)(F)F)c3C2=O)C(=O)N1] | 3-(1-oxo-5-(trifluoromethoxy)- 7-(trifluoromethyl)isoindolin- 2-yl)piperidine-2,6-dione |
| 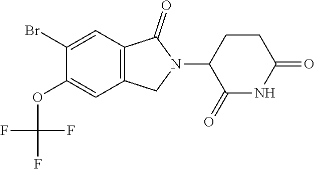 [SMILES: O=C1CCC(N2Cc3cc(OC(F)(F)F)c(Br)cc3C2=O)C(=O)N1] | 3-(6-bromo-1-oxo-5- (trifluoromethoxy)isoindolin- 2-yl)piperidine-2,6-dione |
| 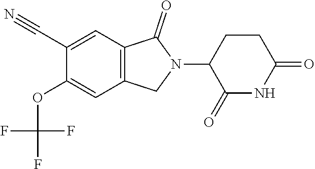 [SMILES: N#Cc1cc2c(cc1OC(F)(F)F)CN(C1CCC(=O)NC1=O)C2=O] | 2-(2,6-dioxopiperidin-3-yl)-3- oxo-6- (trifluoromethoxy)isoindoline- 5-carbonitrile |
| 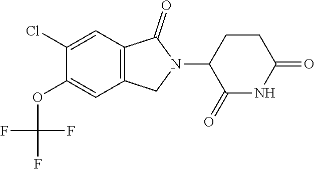 [SMILES: O=C1CCC(N2Cc3cc(OC(F)(F)F)c(Cl)cc3C2=O)C(=O)N1] | 3-(6-chloro-1-oxo-5- (trifluoromethoxy)isoindolin- 2-yl)piperidine-2,6-dione |
| 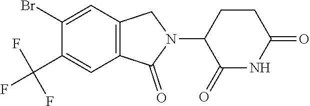 [SMILES: O=C1CCC(N2Cc3cc(Br)c(C(F)(F)F)cc3C2=O)C(=O)N1] | 3-(5-bromo-1-oxo-6- (trifluoromethyl)isoindolin-2- yl)piperidine-2,6-dione |
| 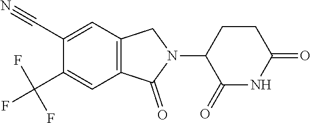 [SMILES: N#Cc1cc2c(cc1C(F)(F)F)C(=O)N(C1CCC(=O)NC1=O)C2] | 2-(2,6-dioxopiperidin-3-yl)-1- oxo-6- (trifluoromethyl)isoindoline-5- carbonitrile |
| 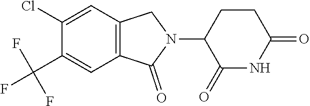 [SMILES: O=C1CCC(N2Cc3cc(Cl)c(C(F)(F)F)cc3C2=O)C(=O)N1] | 3-(5-chloro-1-oxo-6- (trifluoromethyl)isoindolin-2- yl)piperidine-2,6-dione |
| 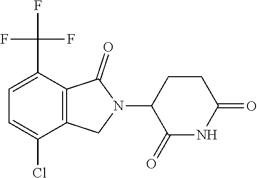 [SMILES: O=C1CCC(N2Cc3c(Cl)ccc(C(F)(F)F)c3C2=O)C(=O)N1] | 3-(4-chloro-1-oxo-7- (trifluoromethyl)isoindolin-2- yl)piperidine-2,6-dione |
| 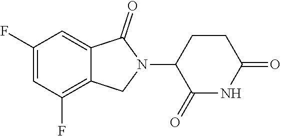 [SMILES: O=C1CCC(N2Cc3c(F)cc(F)cc3C2=O)C(=O)N1] | 3-(4,6-difluoro-1- oxoisoindolin-2-yl)piperidine- 2,6-dione |
| 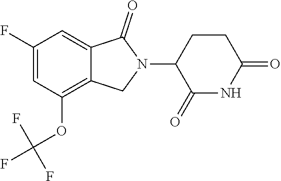 [SMILES: O=C1CCC(N2Cc3c(OC(F)(F)F)cc(F)cc3C2=O)C(=O)N1] | 3-(6-fluoro-1-oxo-4- (trifluoromethoxy)isoindolin- 2-yl)piperidine-2,6-dione |
| 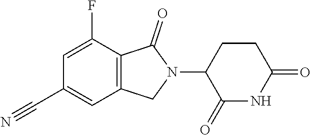 [SMILES: N#Cc1cc(F)c2c(c1)CN(C1CCC(=O)NC1=O)C2=O] | 2-(2,6-dioxopiperidin-3-yl)-7- fluoro-1-oxoisoindoline-5- carbonitrile |
| 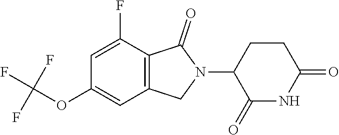 [SMILES: O=C1CCC(N2Cc3cc(OC(F)(F)F)cc(F)c3C2=O)C(=O)N1] | 3-(7-fluoro-1-oxo-5- (trifluoromethoxy)isoindolin- 2-yl)piperidine-2,6-dione |
| 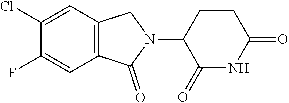 [SMILES: O=C1CCC(N2Cc3cc(Cl)c(F)cc3C2=O)C(=O)N1] | 3-(5-chloro-6-fluoro-1- oxoisoindolin-2-yl)piperidine- 2,6-dione |
| 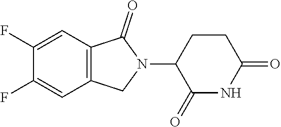 [SMILES: O=C1CCC(N2Cc3cc(F)c(F)cc3C2=O)C(=O)N1] | 3-(5,6-difluoro-1- oxoisoindolin-2-yl)piperidine- 2,6-dione |
| 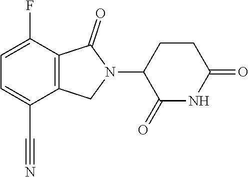 [SMILES: N#Cc1ccc(F)c2c1CN(C1CCC(=O)NC1=O)C2=O] | 2-(2,6-dioxopiperidin-3-yl)-7- fluoro-1-oxoisoindoline-4- carbonitrile |
| 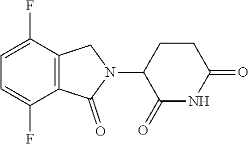 [SMILES: O=C1CCC(N2Cc3c(F)ccc(F)c3C2=O)C(=O)N1] | 3-(4,7-difluoro-1- oxoisoindolin-2-yl)piperidine- 2,6-dione |
| 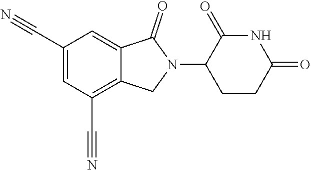 [SMILES: N#Cc1cc(C#N)c2c(c1)C(=O)N(C1CCC(=O)NC1=O)C2] | 2-(2,6-dioxopiperidin-3-yl)-1- oxoisoindoline-4,6- dicarbonitrile |
| 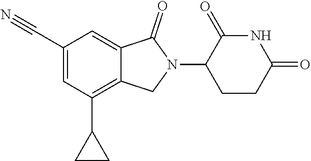 [SMILES: N#Cc1cc2c(c(C3CC3)c1)CN(C1CCC(=O)NC1=O)C2=O] | 7-cyclopropyl-2-(2,6- dioxopiperidin-3-yl)-3- oxoisoindoline-5-carbonitrile |

TABLE 5

| Chemical Structure | Name |
|---|---|
| 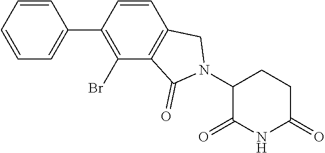 [SMILES: O=C1CCC(N2Cc3ccc(-c4ccccc4)c(Br)c3C2=O)C(=O)N1] | 3-(7-bromo-1-oxo-6- phenylisoindolin-2-yl)piperidine- 2,6-dione |
| 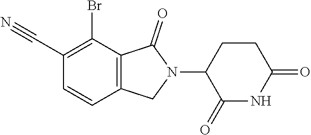 [SMILES: N#Cc1ccc2c(c1Br)C(=O)N(C1CCC(=O)NC1=O)C2] | 4-bromo-2-(2,6-dioxopiperidin-3- yl)-3-oxoisoindoline-5-carbonitrile |
| 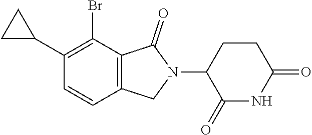 [SMILES: O=C1CCC(N2Cc3ccc(C4CC4)c(Br)c3C2=O)C(=O)N1] | 3-(7-bromo-6-cyclopropyl-1- oxoisoindolin-2-yl)piperidine-2,6- dione |
| 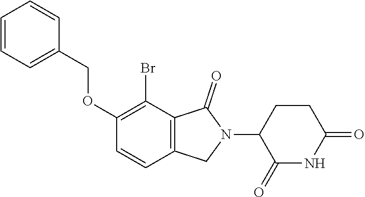 [SMILES: O=C1CCC(N2Cc3ccc(OCc4ccccc4)c(Br)c3C2=O)C(=O)N1] | 3-(6-(benzyloxy)-7-bromo-1- oxoisoindolin-2-yl)piperidine-2,6- dione |
| 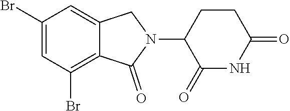 [SMILES: O=C1CCC(N2Cc3cc(Br)cc(Br)c3C2=O)C(=O)N1] | 3-(5,7-dibromo-1-oxoisoindolin-2- yl)piperidine-2,6-dione |
| 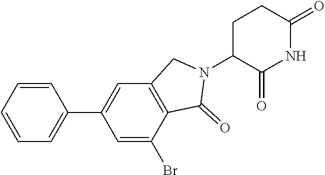 [SMILES: O=C1CCC(N2Cc3cc(-c4ccccc4)cc(Br)c3C2=O)C(=O)N1] | 3-(7-bromo-1-oxo-5- phenylisoindolin-2-yl)piperidine- 2,6-dione |
| 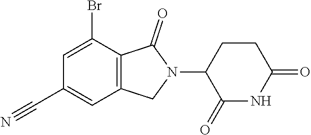 [SMILES: N#Cc1cc(Br)c2c(c1)CN(C1CCC(=O)NC1=O)C2=O] | 7-bromo-2-(2,6-dioxopiperidin-3- yl)-1-oxoisoindoline |
| 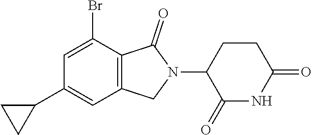 [SMILES: O=C1CCC(N2Cc3cc(C4CC4)cc(Br)c3C2=O)C(=O)N1] | 3-(7-bromo-5-cyclopropyl-1- oxoisoindolin-2-yl)piperidine-2,6- dione |
| 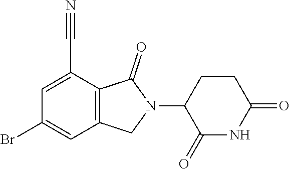 [SMILES: N#Cc1cc(Br)cc2c1C(=O)N(C1CCC(=O)NC1=O)C2] | 6-bromo-2-(2,6-dioxopiperidin-3- yl)-3-oxoisoindoline-4-carbonitrile |
| 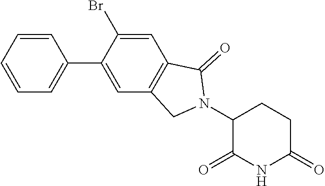 [SMILES: O=C1CCC(N2Cc3cc(-c4ccccc4)c(Br)cc3C2=O)C(=O)N1] | 3-(6-bromo-1-oxo-5- phenylisoindolin-2-yl)piperidine- 2,6-dione |
| 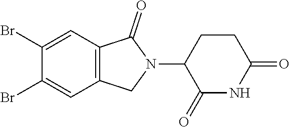 [SMILES: O=C1CCC(N2Cc3cc(Br)c(Br)cc3C2=O)C(=O)N1] | 3-(5,6-dibromo-1-oxoisoindolin-2- yl)piperidine-2,6-dione |
| 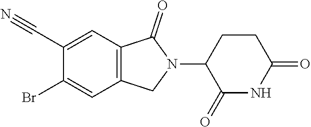 [SMILES: N#Cc1cc2c(cc1Br)CN(C1CCC(=O)NC1=O)C2=O] | 6-bromo-2-(2,6-dioxopiperidin-3- yl)-3-oxoisoindoline-5-carbonitrile |
| 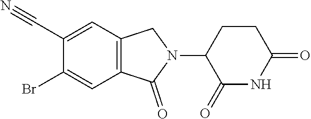 [SMILES: N#Cc1cc2c(cc1Br)C(=O)N(C1CCC(=O)NC1=O)C2] | 6-bromo-2-(2,6-dioxopiperidin-3- yl)-1-oxoisoindoline-5-carbonitrile |
| 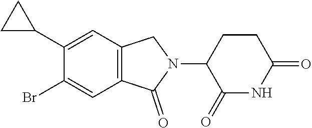 [SMILES: O=C1CCC(N2Cc3cc(C4CC4)c(Br)cc3C2=O)C(=O)N1] | 3-(6-bromo-5-cyclopropyl-1- oxoisoindolin-2-yl)piperidine-2,6- dione |
| 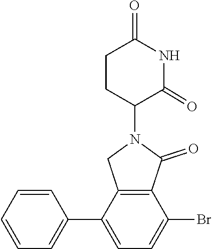 [SMILES: O=C1CCC(N2Cc3c(-c4ccccc4)ccc(Br)c3C2=O)C(=O)N1] | 3-(7-bromo-1-oxo-4- phenylisoindolin-2-yl)piperidine- 2,6-dione |
| 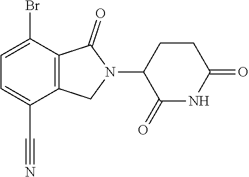 [SMILES: N#Cc1ccc(Br)c2c1CN(C1CCC(=O)NC1=O)C2=O] | 7-bromo-2-(2,6-dioxopiperidin-3- yl)-1-oxoisoindoline-4-carbonitrile |
| 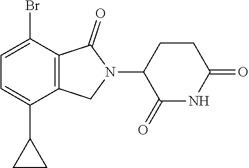 [SMILES: O=C1CCC(N2Cc3c(C4CC4)ccc(Br)c3C2=O)C(=O)N1] | 3-(7-bromo-4-cyclopropyl-1- oxoisoindolin-2-yl)piperidine-2,6- dione |
| 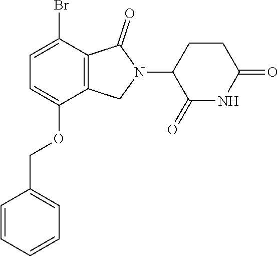 [SMILES: O=C1CCC(N2Cc3c(OCc4ccccc4)ccc(Br)c3C2=O)C(=O)N1] | 3-(4-benzyloxy)-7-bromo-1- oxoisoindolin-2-yl)piperidine-2,6-dione |
| 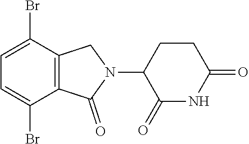 [SMILES: O=C1CCC(N2Cc3c(Br)ccc(Br)c3C2=O)C(=O)N1] | 3-(4,7-dibromo-1-oxoisoindolin-2- yl)piperidine-2,6-dione |
| 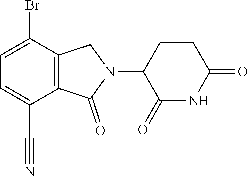 [SMILES: N#Cc1ccc(Br)c2c1C(=O)N(C1CCC(=O)NC1=O)C2] | 7-bromo-2-(2,6-dioxopiperidin-3- yl)-3-oxoisoindoline-4-carbonitrile |
| 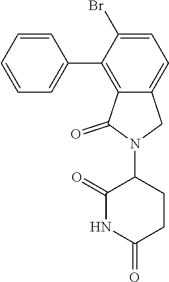 [SMILES: O=C1CCC(N2Cc3ccc(Br)c(-c4ccccc4)c3C2=O)C(=O)N1] | 3-(6-bromo-1-oxo-7- phenylisoindolin-2-yl)piperidine- 2,6-dione |
| 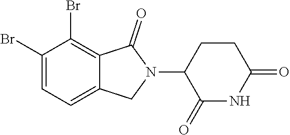 [SMILES: O=C1CCC(N2Cc3ccc(Br)c(Br)c3C2=O)C(=O)N1] | 3-(6,7-dibromo-1-oxoisoindolin-2- yl)piperidine-2,6-dione |
| 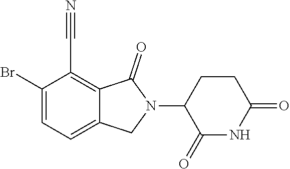 [SMILES: N#Cc1c(Br)ccc2c1C(=O)N(C1CCC(=O)NC1=O)C2] | 5-bromo-2-(2,6-dioxopiperidin-3- yl)-3-oxoisoindoline-4-carbonitrile |
| 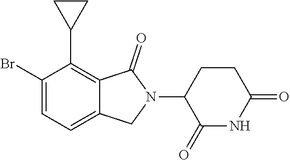 [SMILES: O=C1CCC(N2Cc3ccc(Br)c(C4CC4)c3C2=O)C(=O)N1] | 3-(6-bromo-7-cyclopropyl-1- oxoisoindolin-2-yl)piperidine-2,6- dione |
| 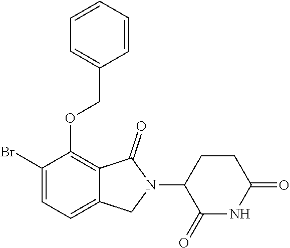 [SMILES: O=C1CCC(N2Cc3ccc(Br)c(OCc4ccccc4)c3C2=O)C(=O)N1] | 3-(7-(benzyloxy)-6-bromo-1- oxoisoindolin-2-yl)piperidine-2,6- dione |
| 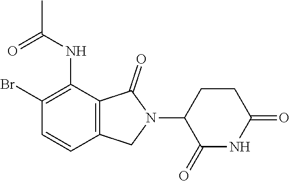 [SMILES: CC(=O)Nc1c(Br)ccc2c1C(=O)N(C1CCC(=O)NC1=O)C2] | N-(5-bromo-2-(2,6-dioxopiperidin- 3-yl)-3-oxoisoindolin-4- yl)acetamide |
| 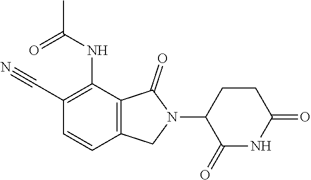 [SMILES: CC(=O)Nc1c(C#N)ccc2c1C(=O)N(C1CCC(=O)NC1=O)C2] | N-(5-cyano-2-(2,6-dioxopiperidin- 3-yl)-3-oxoisoindolin-4- yl)acetamide |
| 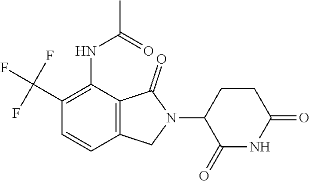 [SMILES: CC(=O)Nc1c(C(F)(F)F)ccc2c1C(=O)N(C1CCC(=O)NC1=O)C2] | N-(2-(2,6-dioxopiperidin-3-yl)-3- oxo-5-(trifluoromethyl)isoindolin- 4-yl)acetamide |
| 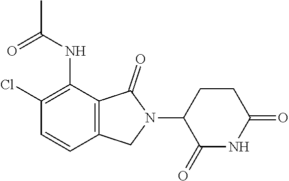 [SMILES: CC(=O)Nc1c(Cl)ccc2c1C(=O)N(C1CCC(=O)NC1=O)C2] | N-(5-chloro-2-(2,6-dioxopiperidin- 3-yl)-3-oxoisoindolin-4- yl)acetamide |
| 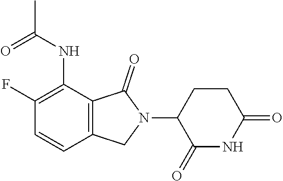 [SMILES: CC(=O)Nc1c(F)ccc2c1C(=O)N(C1CCC(=O)NC1=O)C2] | N-(2-(2,6-dioxopiperidin-3-yl)-5- fluoro-3-oxoisoindolin-4- yl)acetamide |
| 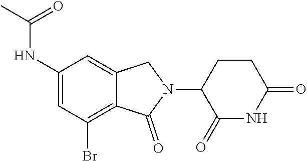 [SMILES: CC(=O)Nc1cc(Br)c2c(c1)CN(C1CCC(=O)NC1=O)C2=O] | N-(7-bromo-2-(2,6-dioxopiperidin- 3-yl)-1-oxoisoindolin-5- yl)acetamide |
| 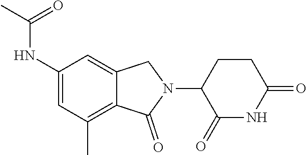 [SMILES: CC(=O)Nc1cc(C)c2c(c1)CN(C1CCC(=O)NC1=O)C2=O] | N-(2-(2,6-dioxopiperidin-3-yl)-7- methyl-1-oxoisoindolin-5- yl)acetamide |
| 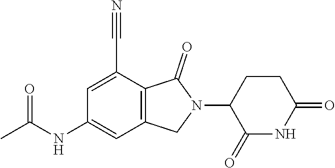 [SMILES: CC(=O)Nc1cc(C#N)c2c(c1)CN(C1CCC(=O)NC1=O)C2=O] | N-(7-cyano-2-(2,6-dioxopiperidin- 3-yl)-1-oxoisoindolin-5- yl)acetamide |
| 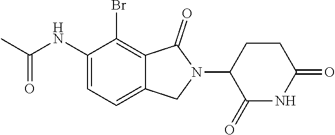 [SMILES: CC(=O)Nc1ccc2c(c1Br)C(=O)N(C1CCC(=O)NC1=O)C2] | N-(4-bromo-2-(2,6-dioxopiperidin- 3-yl)-3-oxoisoindolin-5- yl)acetamide |
| 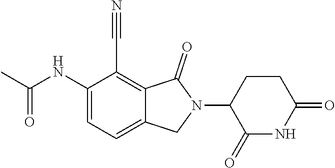 [SMILES: CC(=O)Nc1ccc2c(c1C#N)C(=O)N(C1CCC(=O)NC1=O)C2] | N-(4-cyano-2-(2,6-dioxopiperidin- 3-yl)-3-oxoisoindolin-5- yl)acetamide |
| 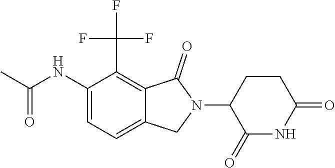 [SMILES: CC(=O)Nc1ccc2c(c1C(F)(F)F)C(=O)N(C1CCC(=O)NC1=O)C2] | N-(2-(2,6-dioxopiperidin-3-yl)-4- methyl-3-oxoisoindolin-5- yl)acetamide |
| 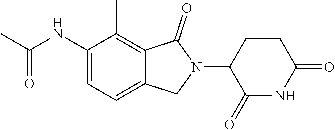 [SMILES: CC(=O)Nc1ccc2c(c1C)C(=O)N(C1CCC(=O)NC1=O)C2] | N-(2-(2,6-dioxopiperidin-3-yl)-4- methyl-3-oxoisoindolin-5- yl)acetamide |
| 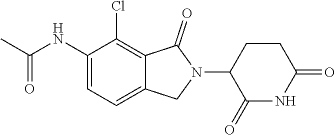 [SMILES: CC(=O)Nc1ccc2c(c1Cl)C(=O)N(C1CCC(=O)NC1=O)C2] | N-(4-chloro-2-(2,6-dioxopiperidin- 3-yl)-3-oxoisoindolin-5- yl)acetamide |
| 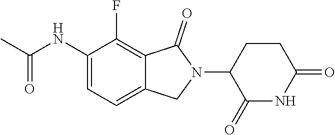 [SMILES: CC(=O)Nc1ccc2c(c1F)C(=O)N(C1CCC(=O)NC1=O)C2] | N-(2-(2,6-dioxopiperidin-3-yl)-4- fluoro-3-oxoisoindolin-5- yl)acetamide |
| 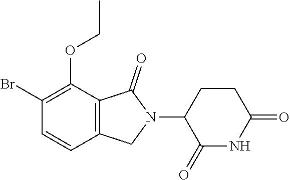 [SMILES: CCOc1c(Br)ccc2c1C(=O)N(C1CCC(=O)NC1=O)C2] | 3-(6-bromo-7-ethoxy-1- oxoisoindolin-2-yl)piperidine-2,6- dione |
| 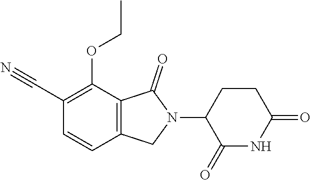 [SMILES: CCOc1c(C#N)ccc2c1C(=O)N(C1CCC(=O)NC1=O)C2] | 2-(2,6-dioxopiperidin-3-yl)-4- ethoxy-3-oxoisoindoline-5- carbonitrile |
| 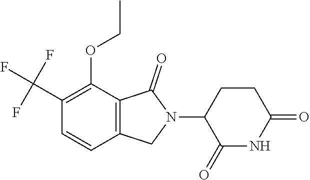 [SMILES: CCOc1c(C(F)(F)F)ccc2c1C(=O)N(C1CCC(=O)NC1=O)C2] | 3-(7-ethoxy-1-oxo-6- (trifluoromethyl)isoindolin-2- yl)piperidine-2,6-dione |
| 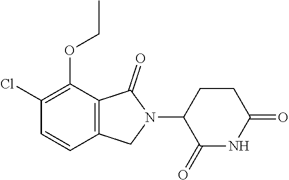 [SMILES: CCOc1c(Cl)ccc2c1C(=O)N(C1CCC(=O)NC1=O)C2] | 3-(6-chloro-7-ethoxy-1- oxoisoindolin-2-yl)piperidine-2,6- dione |
| 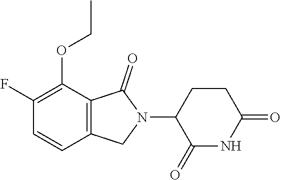 [SMILES: CCOc1c(F)ccc2c1C(=O)N(C1CCC(=O)NC1=O)C2] | 3-(7-ethoxy-6-fluoro-1- oxoisoindolin-2-yl)piperidine-2,6- dione |
| 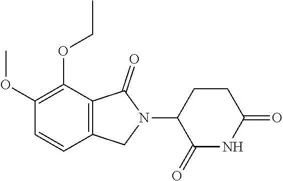 [SMILES: CCOc1c(OC)ccc2c1C(=O)N(C1CCC(=O)NC1=O)C2] | 3-(7-ethoxy-6-methoxy-1- oxoisoindolin-2-yl)piperidine-2,6- dione |
| 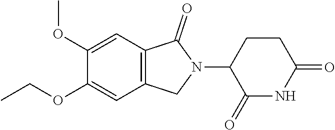 [SMILES: CCOc1cc2c(cc1OC)C(=O)N(C1CCC(=O)NC1=O)C2] | 3-(5-ethoxy-6-methoxy-1- oxoisoindolin-2-yl)piperidine-2,6-dione |
| 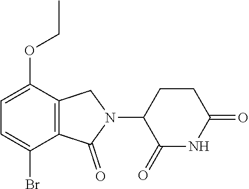 [SMILES: CCOc1ccc(Br)c2c1CN(C1CCC(=O)NC1=O)C2=O] | 3-(7-bromo-4-ethoxy-1- oxoisoindolin-2-yl)piperidine-2,6- dione |
| 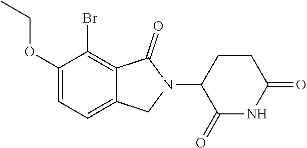 [SMILES: CCOc1ccc2c(c1Br)C(=O)N(C1CCC(=O)NC1=O)C2] | 3-(7-bromo-6-ethoxy-1- oxoisoindolin-2-yl)piperidine-2,6- dione |
| 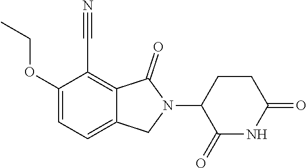 [SMILES: CCOc1ccc2c(c1C#N)C(=O)N(C1CCC(=O)NC1=O)C2] | 2-(2,6-dioxopiperidin-3-yl)-5- ethoxy-3-oxoisoindoline-4- carbonitrile |
| 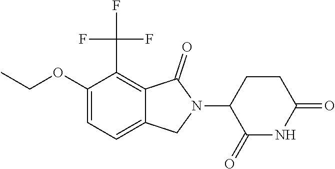 [SMILES: CCOc1ccc2c(c1C(F)(F)F)C(=O)N(C1CCC(=O)NC1=O)C2] | 3-(6-ethoxy-1-oxo-7- (trifluoromethy)isoindolin-2- yl)piperidine-2,6-dione |
| 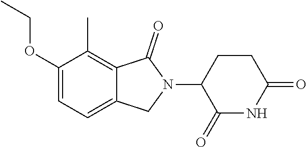 [SMILES: CCOc1ccc2c(c1C)C(=O)N(C1CCC(=O)NC1=O)C2] | 3-(6-ethoxy-7-methyl-1- oxoisoindolin-2-yl)piperidine-2,6- dione |
| 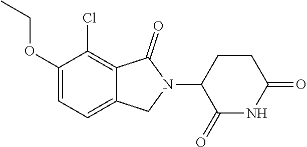 [SMILES: CCOc1ccc2c(c1Cl)C(=O)N(C1CCC(=O)NC1=O)C2] | 3-(7-chloro-6-ethoxy-1- oxoisoindolin-2-yl)piperidine-2,6- dione |
| 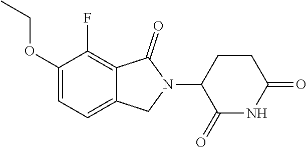 [SMILES: CCOc1ccc2c(c1F)C(=O)N(C1CCC(=O)NC1=O)C2] | 3-(6-ethoxy-7-fluoro-1- oxoisoindolin-2-yl)piperidine-2,6- dione |
| 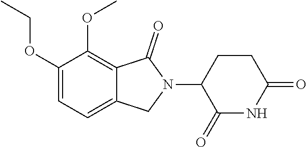 [SMILES: CCOc1ccc2c(c1OC)C(=O)N(C1CCC(=O)NC1=O)C2] | 3-(6-ethoxy-7-methoxy-1- oxoisoindolin-2-yl)piperidine-2,6- dione |
| 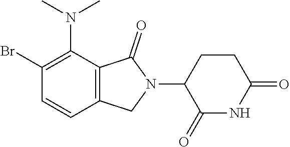 [SMILES: CN(C)c1c(Br)ccc2c1C(=O)N(C1CCC(=O)NC1=O)C2] | 3-(6-bromo-7-(dimethylamino)-1- oxoisoindolin-2-yl)piperidine-2,6- dione |
| 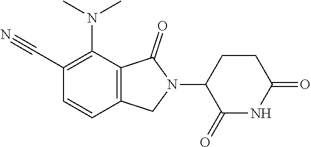 [SMILES: CN(C)c1c(C#N)ccc2c1C(=O)N(C1CCC(=O)NC1=O)C2] | 4-(dimethylamino)-2-(2,6- dioxopiperidin-3-yl)-3- oxoisoindolin-5-carbonitrile |
| 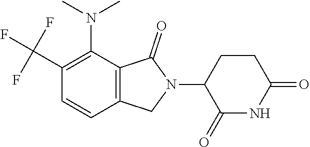 [SMILES: CN(C)c1c(C(F)(F)F)ccc2c1C(=O)N(C1CCC(=O)NC1=O)C2] | 3-(7-(dimethylaino)-1-oxo-6- (trifluoromethyl)isoindolin-2- yl)piperidine-2,6-dione |
| 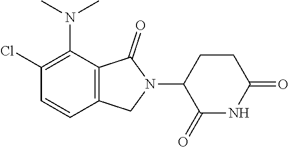 [SMILES: CN(C)c1c(Cl)ccc2c1C(=O)N(C1CCC(=O)NC1=O)C2] | 3-(6-chloro-7-(dimethylamino)-1- oxoisoindolin-2-yl)piperidine-2,6- dione |
| 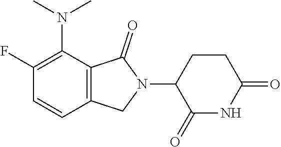 [SMILES: CN(C)c1c(F)ccc2c1C(=O)N(C1CCC(=O)NC1=O)C2] | 3-(7-(dimethylamino)-6-fluoro-1- oxoisoindolin-2-yl)piperidine-2,6- dione |
| 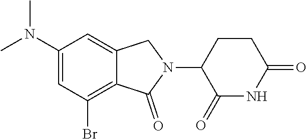 [SMILES: CN(C)c1cc(Br)c2c(c1)CN(C1CCC(=O)NC1=O)C2=O] | 3-(7-bromo-5-(dimethylamino)-1- oxoisoindolin-2-yl)piperidine-2,6- dione |
| 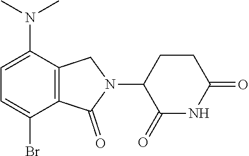 [SMILES: CN(C)c1ccc(Br)c2c1CN(C1CCC(=O)NC1=O)C2=O] | 3-(7-bromo-4-(dimethylamino)-1- oxoisoindolin-2-yl)piperidine-2,6-dione |
| 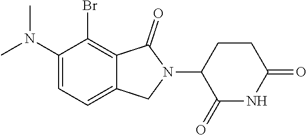 [SMILES: CN(C)c1ccc2c(c1Br)C(=O)N(C1CCC(=O)NC1=O)C2] | 3-(7-bromo-6-(dimethylamino)-1- oxoisoindolin-2-yl)piperidine-2,6- dione |
| 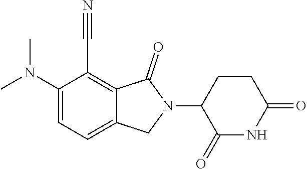 [SMILES: CN(C)c1ccc2c(c1C#N)C(=O)N(C1CCC(=O)NC1=O)C2] | 5-(dimethylamino)-2-(2,6- dioxopiperidin-3-yl)-3- oxoisoindoline-4-carbonitrile |
| 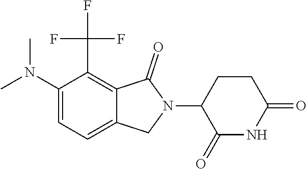 [SMILES: CN(C)c1ccc2c(c1C(F)(F)F)C(=O)N(C1CCC(=O)NC1=O)C2] | 3-(6-(dimethylamino)-1-oxo-7- (trifluoromethyl)isoindolin-2- yl)piperidine-2,6-dione |
| 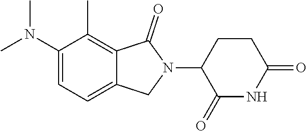 [SMILES: Cc1c(N(C)C)ccc2c1C(=O)N(C1CCC(=O)NC1=O)C2] | 3-(6-(dimethylamino)-7-methyl-1- oxoisoindolin-2-yl)piperidine-2,6- dione |
| 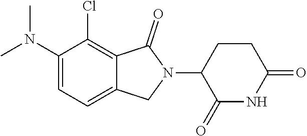 [SMILES: CN(C)c1ccc2c(c1Cl)C(=O)N(C1CCC(=O)NC1=O)C2] | 3-(7-chloro-6-(dimethylamino)-1- oxoisoindolin-2-yl)piperidine-2,6- dione |
| 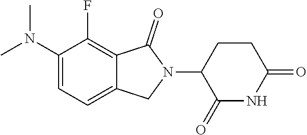 [SMILES: CN(C)c1ccc2c(c1F)C(=O)N(C1CCC(=O)NC1=O)C2] | 3-(6-(dimethylamino)-7-fluoro-1- oxoisoindolin-2-yl)piperidine-2,6- dione |
| 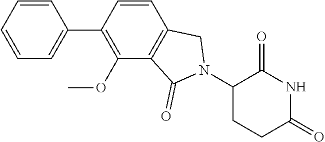 [SMILES: COc1c(-c2ccccc2)ccc2c1C(=O)N(C1CCC(=O)NC1=O)C2] | 3-(7-methoxy-1-oxo-6- phenylisoindolin-2-yl)piperidine- 2,6-dione |
| 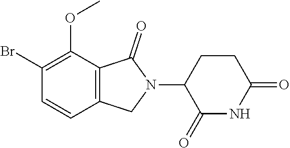 [SMILES: COc1c(Br)ccc2c1C(=O)N(C1CCC(=O)NC1=O)C2] | 3-(6-bromo-7-methoxy-1- oxoisoindolin-2-yl)piperidine-2,6- dione |
| 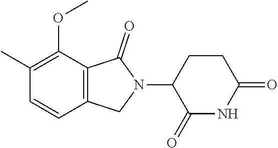 [SMILES: COc1c(C)ccc2c1C(=O)N(C1CCC(=O)NC1=O)C2] | 3-(7-methoxy-6-methyl-1- oxoisoindolin-2-yl)piperidine-2,6- dione |
| 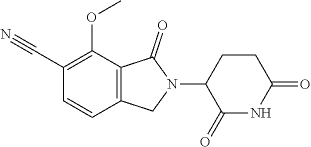 [SMILES: COc1c(C#N)ccc2c1C(=O)N(C1CCC(=O)NC1=O)C2] | 2-(2,6-dioxopiperidin-3-yl)-4- methoxy-3-oxoisoindoline-5- carbonitrile |
| 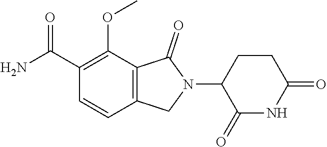 [SMILES: COc1c(C(N)=O)ccc2c1C(=O)N(C1CCC(=O)NC1=O)C2] | 2-(2,6-dioxopiperidin-3-yl)-4- methoxy-3-oxoisoindoline-5- carboxamide |
| 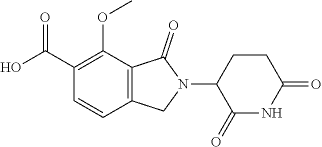 [SMILES: COc1c(C(=O)O)ccc2c1C(=O)N(C1CCC(=O)NC1=O)C2] | 2-(2,6-dioxopiperidin-3-yl)-4- methoxy-3-oxoisoindoline-5- carboxylic acid |
| 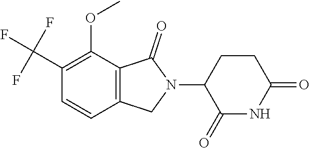 [SMILES: COc1c(C(F)(F)F)ccc2c1C(=O)N(C1CCC(=O)NC1=O)C2] | 3-(7-methoxy-1-oxo-6- (trifluoromethyl)isoindolin-2- yl)piperidine-2,6-dione |
| 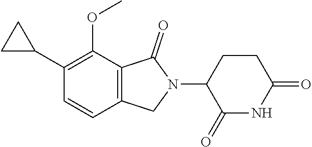 [SMILES: COc1c(C2CC2)ccc2c1C(=O)N(C1CCC(=O)NC1=O)C2] | 3-(6-cyclopropyl-7-methoxy-1- oxoisoindolin-2-yl)piperidine-2,6- dione |
| 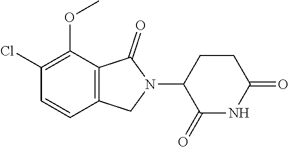 [SMILES: COc1c(Cl)ccc2c1C(=O)N(C1CCC(=O)NC1=O)C2] | 3-(6-chloro-7-methoxy-1- oxoisoindolin-2-yl)piperidine-2,6- dione |
| 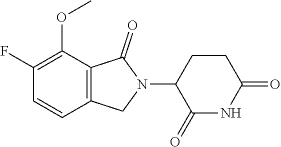 [SMILES: COc1c(F)ccc2c1C(=O)N(C1CCC(=O)NC1=O)C2] | 3-(6-fluoro-7-methoxy-1- oxoisoindolin-2-yl)piperidine-2,6- dione |
| 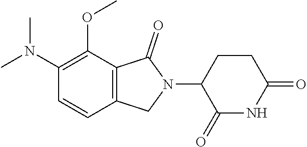 [SMILES: COc1c(N(C)C)ccc2c1C(=O)N(C1CCC(=O)NC1=O)C2] | 3-(6-(dimethylamino)-7-methoxy- 1-oxoisoindolin-2-yl)piperidine- 2,6-dione |
| 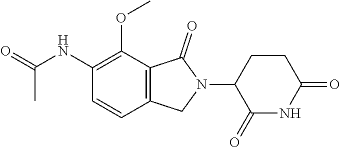 [SMILES: COc1c(NC(C)=O)ccc2c1C(=O)N(C1CCC(=O)NC1=O)C2] | N-(2-(2,6-dioxopiperidin-3-yl)-4- methoxy-3-oxoisoindolin-5- yl)acetamide |
| 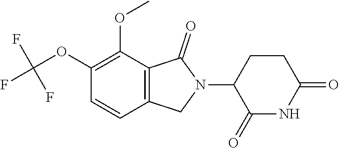 [SMILES: COc1c(OC(F)(F)F)ccc2c1C(=O)N(C1CCC(=O)NC1=O)C2] | 3-(7-methoxy-1-oxo-6- (trifluoromethoxy)isoindolin-2- yl)piperidine-2,6-dione |
| 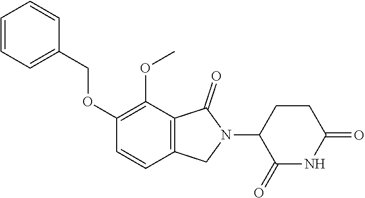 [SMILES: COc1c(OCc2ccccc2)ccc2c1C(=O)N(C1CCC(=O)NC1=O)C2] | 3-(6-(benzyloxy)-7-methoxy-1- oxoisoindolin-2-yl)piperidine-2,6- dione |
| 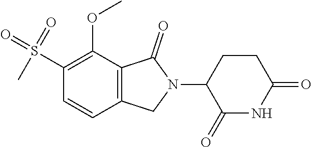 [SMILES: COc1c(S(C)(=O)=O)ccc2c1C(=O)N(C1CCC(=O)NC1=O)C2] | 3-(7-methoxy-6-(methylsulfonyl)- 1-oxoisoindolin-2-yl)piperidine- 2,6-dione |
| 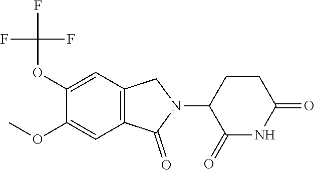 [SMILES: COc1cc2c(cc1OC(F)(F)F)CN(C1CCC(=O)NC1=O)C2=O] | 3-(6-methoxy-1-oxo-5- (trifluoromethoxy)isoindolin-2- yl)piperidine-2,6-dione |
| 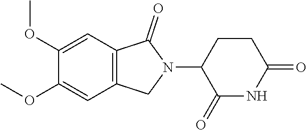 [SMILES: COc1cc2c(cc1OC)C(=O)N(C1CCC(=O)NC1=O)C2] | 3-(5,6-dimethoxy-1-oxoisoindolin- 2-yl)piperidine-2,6-dione |
| 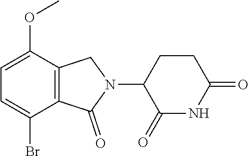 [SMILES: COc1ccc(Br)c2c1CN(C1CCC(=O)NC1=O)C2=O] | 3-(7-bromo-4-methoxy-1- oxoisoindolin-2-yl)piperidine-2,6- dione |
| 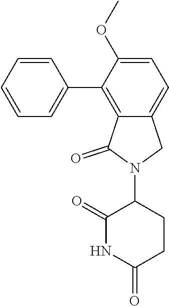 [SMILES: COc1ccc2c(c1-c1ccccc1)C(=O)N(C1CCC(=O)NC1=O)C2] | 3-(6-methooxy-1-oxo-7- phenylisoindolin-2-yl)piperidine- 2,6-dione |
| 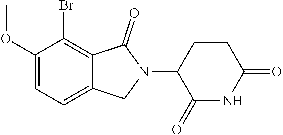 [SMILES: COc1ccc2c(c1Br)C(=O)N(C1CCC(=O)NC1=O)C2] | 3-(7-bromo-6-methoxy-1- oxoisoindolin-2-yl)piperidine-2,6- dione |
| 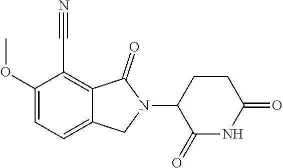 [SMILES: COc1ccc2c(c1C#N)C(=O)N(C1CCC(=O)NC1=O)C2] | 2-(2,6-dioxopiperidin-3-yl)-5- methoxy-3-oxoisoindoline-4- carbonitrile |
| 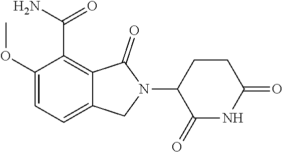 [SMILES: COc1ccc2c(c1C(N)=O)C(=O)N(C1CCC(=O)NC1=O)C2] | 2-(2,6-dioxopiperidin-3-yl)-5- methoxy-3-oxoisoindoline-4- carboxamide |
| 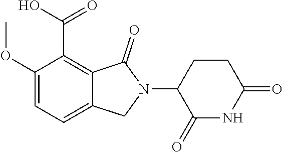 [SMILES: COc1ccc2c(c1C(=O)O)C(=O)N(C1CCC(=O)NC1=O)C2] | 2-(2,6-dioxopiperidin-3-yl)-5- methoxy-3-oxoisoindoline-4- carboxylic acid |
| 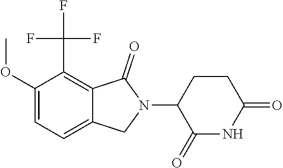 [SMILES: COc1ccc2c(c1C(F)(F)F)C(=O)N(C1CCC(=O)NC1=O)C2] | 3-(6-methoxy-1-oxo-7- (trifluoromethyl)isoindolin-2- yl)piperidine-2,6-dione |
| 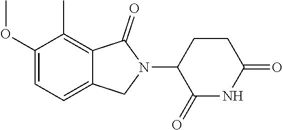 [SMILES: COc1ccc2c(c1C)C(=O)N(C1CCC(=O)NC1=O)C2] | 3-(6-methoxy-7-methyl-1- oxoisoindolin-2-yl)piperidine-2,6- dione |
| 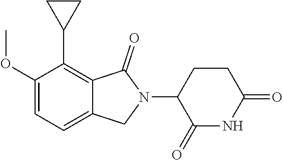 [SMILES: COc1ccc2c(c1C1CC1)C(=O)N(C1CCC(=O)NC1=O)C2] | 3-(7-cyclopropyl-6-methoxy-1- oxoisoindolin-2-yl)piperidine-2,6- dione |
| 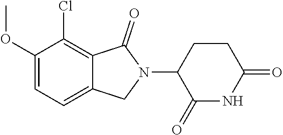 [SMILES: COc1ccc2c(c1Cl)C(=O)N(C1CCC(=O)NC1=O)C2] | 3-(7-chloro-6-methoxy-1- oxoisoindolin-2-yl)piperidine-2,6- dione |
| 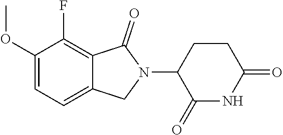 [SMILES: COc1ccc2c(c1F)C(=O)N(C1CCC(=O)NC1=O)C2] | 3-(7-fluoro-6-methoxy-1- oxoisoindolin-2-yl)piperidine-2,6- dione |
| 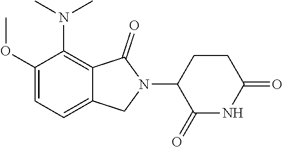 [SMILES: COc1ccc2c(c1N(C)C)C(=O)N(C1CCC(=O)NC1=O)C2] | 3-(7-(dimethylamino)-6-methoxy- 1-oxoisoindolin-2-yl)piperidine- 2,6-dione |
| 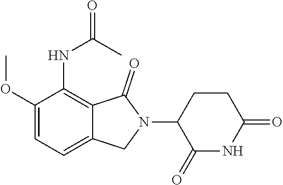 [SMILES: COc1ccc2c(c1NC(C)=O)C(=O)N(C1CCC(=O)NC1=O)C2] | N-(2-(2,6-dioxopiperidin-3-yl)-5- methoxy-3-oxoisoindolin-4- yl)acetamide |
| 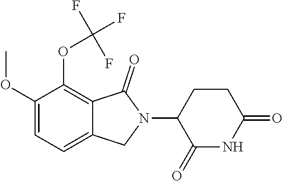 [SMILES: COc1ccc2c(c1OC(F)(F)F)C(=O)N(C1CCC(=O)NC1=O)C2] | 3-(6-methoxy-1-oxo-7- (trifluoromethoxy)isoindolin-2- yl)piperidine-2,6-dione |
| 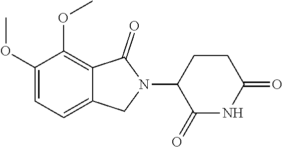 [SMILES: COc1ccc2c(c1OC)C(=O)N(C1CCC(=O)NC1=O)C2] | 3-(6,7-dimethoxy-1-oxoisoindolin- 2-yl)piperidine-2,6-dione |
| 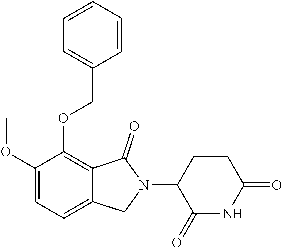 [SMILES: COc1ccc2c(c1OCc1ccccc1)C(=O)N(C1CCC(=O)NC1=O)C2] | 3-(7-(benzyloxy)-6-methoxy-1- oxoisoindolin-2-yl)piperidine-2,6- dione |
| 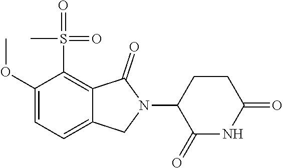 [SMILES: COc1ccc2c(c1S(C)(=O)=O)C(=O)N(C1CCC(=O)NC1=O)C2] | 3-(6-methoxy-7-(methylsulfonyl)- 1-oxoisoindolin-2-yl)piperidine- 2,6-dione |
| 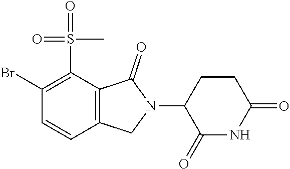 [SMILES: CS(=O)(=O)c1c(Br)ccc2c1C(=O)N(C1CCC(=O)NC1=O)C2] | 3-(6-bromo-7-(methylsulfonyl)-1- oxoisoindolin-2-yl)piperidine-2,6- dione |
| 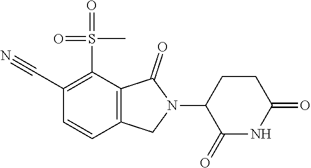 [SMILES: CS(=O)(=O)c1c(C#N)ccc2c1C(=O)N(C1CCC(=O)NC1=O)C2] | 2-(2,6-dioxopiperidin-3-yl)-4- (methylsulfonyl)-3-oxoisoindoline- 5-carbonitrile |
| 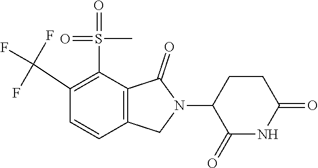 [SMILES: CS(=O)(=O)c1c(C(F)(F)F)ccc2c1C(=O)N(C1CCC(=O)NC1=O)C2] | 3-(7-(methylsulfonyl)-1-oxo-6- (trifluoromethyl)isoindolin-2- yl)piperidine-2,6-dione |
| 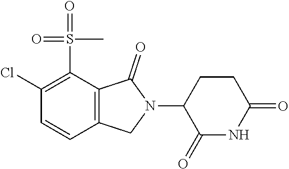 [SMILES: CS(=O)(=O)c1c(Cl)ccc2c1C(=O)N(C1CCC(=O)NC1=O)C2] | 3-(6-chloro-7-(methylsulfonyl)-1- oxoisoindolin-2-yl)piperidine-2,6- dione |
| 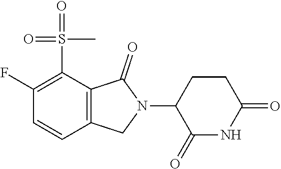 [SMILES: CS(=O)(=O)c1c(F)ccc2c1C(=O)N(C1CCC(=O)NC1=O)C2] | 3-(6-fluoro-7-(methylsulfonyl)-1- oxoisoindolin-2-yl)piperidine-2,6- dione |
| 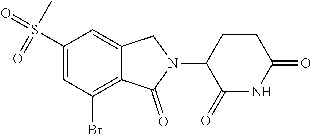 [SMILES: CS(=O)(=O)c1cc(Br)c2c(c1)CN(C1CCC(=O)NC1=O)C2=O] | 3-(7-bromo-5-(methylsulfonyl)-1- oxoisoindolin-2-yl)piperidine-2,6- dione |
| 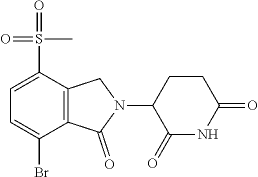 [SMILES: CS(=O)(=O)c1ccc(Br)c2c1CN(C1CCC(=O)NC1=O)C2=O] | 3-(7-bromo-4-(methylsulfonyl)-1- oxoisoindolin-2-yl)piperidine-2,6- dione |
| 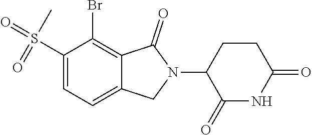 [SMILES: CS(=O)(=O)c1ccc2c(c1Br)C(=O)N(C1CCC(=O)NC1=O)C2] | 3-(7-bromo-6-(methylsulfonyl)-1- oxoisoindolin-2-yl)piperidine-2,6- dione |
| 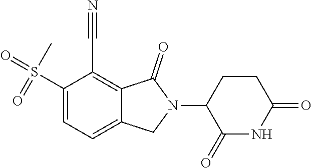 [SMILES: CS(=O)(=O)c1ccc2c(c1C#N)C(=O)N(C1CCC(=O)NC1=O)C2] | 2-(2,6-dioxopiperidin-3-yl)-5- (methylsulfonyl)-3-oxoisoindoline- 4-carbonitrile |
| 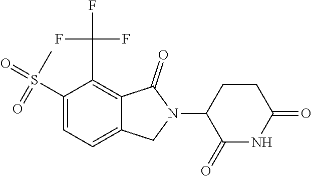 [SMILES: CS(=O)(=O)c1ccc2c(c1C(F)(F)F)C(=O)N(C1CCC(=O)NC1=O)C2] | 3-(6-(methylsulfonyl)-1-oxo-7- (trifluoromethyl)isoindolin-2- yl)piperidine-2,6-dione |
| 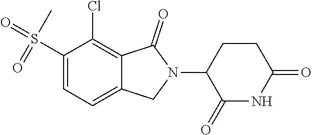 [SMILES: CS(=O)(=O)c1ccc2c(c1Cl)C(=O)N(C1CCC(=O)NC1=O)C2] | 3-(7-chloro-6-(methylsulfonyl)-1- oxoisoindolin-2-yl)piperidine-2,6- dione |
| 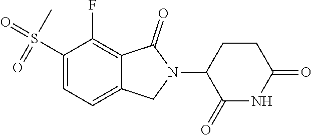 [SMILES: CS(=O)(=O)c1ccc2c(c1F)C(=O)N(C1CCC(=O)NC1=O)C2] | 3-(7-fluoro-6-(methylsulfonyl)-1- oxoisoindolin-2-yl)piperidine-2,6- dione |
| 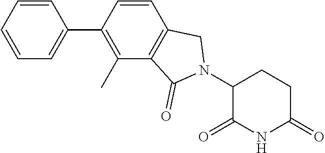 [SMILES: Cc1c(-c2ccccc2)ccc2c1C(=O)N(C1CCC(=O)NC1=O)C2] | 3-(7-methyl-1-oxo-6- phenylisoindolin-2-yl)piperidine- 2,6-dione |
| 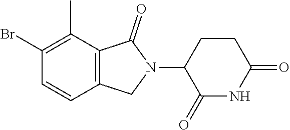 [SMILES: Cc1c(Br)ccc2c1C(=O)N(C1CCC(=O)NC1=O)C2] | 3-(6-bromo-7-methyl-1- oxoisoindolin-2-yl)piperidine-2,6- dione |
| 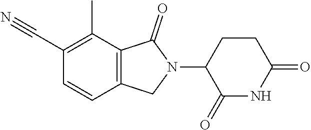 [SMILES: Cc1c(C#N)ccc2c1C(=O)N(C1CCC(=O)NC1=O)C2] | 2-(2,6-dioxopiperidin-3-yl)-4- methyl-3-oxoisoindoline-5- carbonitrile |
| 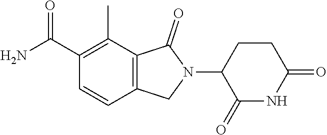 [SMILES: Cc1c(C(N)=O)ccc2c1C(=O)N(C1CCC(=O)NC1=O)C2] | 2-(2,6-dioxopiperidin-3-yl)-4- methyl-3-oxoisoindoline-5- carboxamide |
| 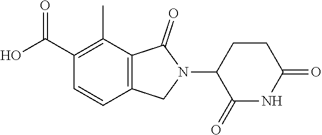 [SMILES: Cc1c(C(=O)O)ccc2c1C(=O)N(C1CCC(=O)NC1=O)C2] | 2-(2,6-dioxopiperidin-3-yl)-4- methyl-3-oxoisoindoline-5- carboxylic acid |
| 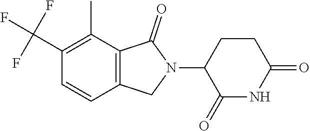 [SMILES: Cc1c(C(F)(F)F)ccc2c1C(=O)N(C1CCC(=O)NC1=O)C2] | 3-(7-methyl-1-oxo-6- (trifluoromethyl)isoindolin-2- yl)piperidine-2,6-dione |
| 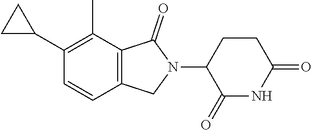 [SMILES: Cc1c(C2CC2)ccc2c1C(=O)N(C1CCC(=O)NC1=O)C2] | 3-(6-cyclopropyl-7-methyl-1- oxoisoindolin-2-yl)piperidine-2,6- dione |
| 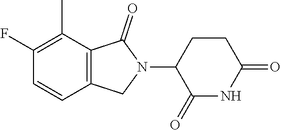 [SMILES: Cc1c(F)ccc2c1C(=O)N(C1CCC(=O)NC1=O)C2] | 3-(6-fluoro-7-methyl-1- oxoisoindolin-2-yl)piperidine-2,6- dione |
| 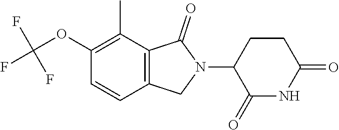 [SMILES: Cc1c(OC(F)(F)F)ccc2c1C(=O)N(C1CCC(=O)NC1=O)C2] | 3-(7-methyl-1-oxo-6- (trifluoromethoxy)isoindolin-2- yl)piperidine-2,6-dione |
| 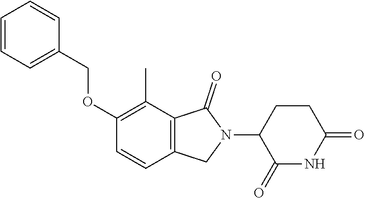 [SMILES: Cc1c(OCc2ccccc2)ccc2c1C(=O)N(C1CCC(=O)NC1=O)C2] | 3-(6-(benzyloxy)-7-methyl-1- oxoisoindolin-2-yl)piperidine-2,6- dione |
| 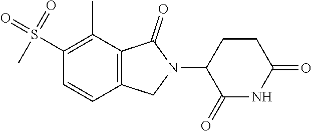 [SMILES: Cc1c(S(C)(=O)=O)ccc2c1C(=O)N(C1CCC(=O)NC1=O)C2] | 3-(7-methyl-6-(methylsulfonyl)-1-oxoisoindolin-2- yl)piperidine |
| 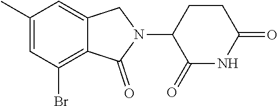 [SMILES: Cc1cc(Br)c2c(c1)CN(C1CCC(=O)NC1=O)C2=O] | 3-(7-bromo-5-methyl-1- oxoisoindolin-2-yl)piperidine-2,6- dione |
| 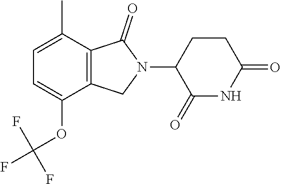 [SMILES: Cc1ccc(OC(F)(F)F)c2c1C(=O)N(C1CCC(=O)NC1=O)C2] | 3-(7-methyl-1-oxo-4- (trifluoromethoxy)isoindolin-2- yl)piperidine-2,6-dione |
| 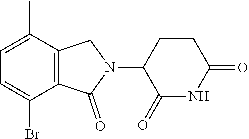 [SMILES: Cc1ccc(Br)c2c1CN(C1CCC(=O)NC1=O)C2=O] | 3-(7-bromo-4-methyl-1- oxoisoindolin-2-yl)piperidine-2,6- dione |
| 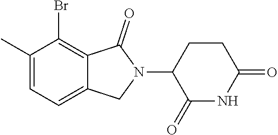 [SMILES: Cc1ccc2c(c1Br)C(=O)N(C1CCC(=O)NC1=O)C2] | 3-(7-bromo-6-methyl-1- oxoisoindolin-2-yl)piperidine-2,6- dione |
| 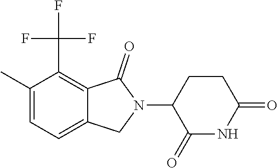 [SMILES: Cc1ccc2c(c1C(F)(F)F)C(=O)N(C1CCC(=O)NC1=O)C2] | 3-(6-methyl-1-oxo-7- (trifluoromethyl)isoindolin-2- yl)piperidine-2,6-dione |
| 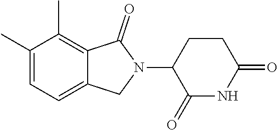 [SMILES: Cc1ccc2c(c1C)C(=O)N(C1CCC(=O)NC1=O)C2] | 3-(6,7-dimethyl-1-oxoisoindolin-2- yl)piperidine-2,6-dione |
| 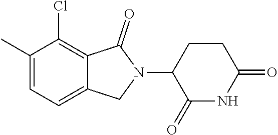 [SMILES: Cc1ccc2c(c1Cl)C(=O)N(C1CCC(=O)NC1=O)C2] | 3-(7-chloro-6-methyl-1- oxoisoindolin-2-yl)piperidine-2,6-dione |
| 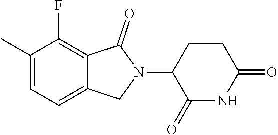 [SMILES: Cc1ccc2c(c1F)C(=O)N(C1CCC(=O)NC1=O)C2] | 3-(7-fluoro-6-methyl-1- oxoisoindolin-2-yl)piperidine-2,6-dione |
| 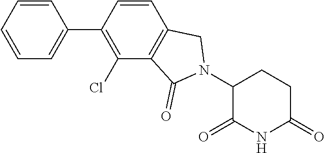 [SMILES: O=C1CCC(N2Cc3ccc(-c4ccccc4)c(Cl)c3C2=O)C(=O)N1] | 3-(7-chloro-1-oxo-6- phenylisoindolin-2-yl)piperidine- 2,6-dione |
| 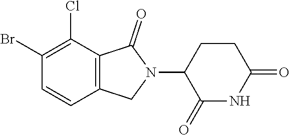 [SMILES: O=C1CCC(N2Cc3ccc(Br)c(Cl)c3C2=O)C(=O)N1] | 3-(6-bromo-7-chloro-1- oxoisoindolin-2-yl)piperidine-2,6-dione |
| 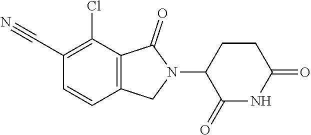 [SMILES: N#Cc1ccc2c(c1Cl)C(=O)N(C1CCC(=O)NC1=O)C2] | 4-chloro-2-(2,6-dioxopiperidin-3- yl)-3-oxoisoindoline-5-carbonitrile |
| 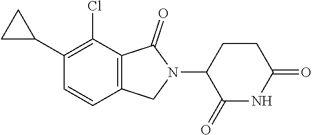 [SMILES: O=C1CCC(N2Cc3ccc(C4CC4)c(Cl)c3C2=O)C(=O)N1] | 3-(7-chloro-6-cyclopropyl-1- oxoisoindolin-2-yl)piperidine-2,6- dione |
| 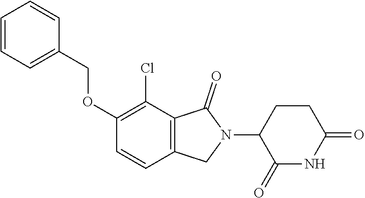 [SMILES: O=C1CCC(N2Cc3ccc(OCc4ccccc4)c(Cl)c3C2=O)C(=O)N1] | 3-(6-(benzyloxy)-7-chloro-1- oxoisoindolin-2-yl)piperidine-2,6- dione |
| 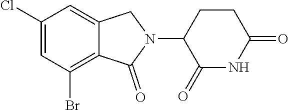 [SMILES: O=C1CCC(N2Cc3cc(Cl)cc(Br)c3C2=O)C(=O)N1] | 3-(7-bromo-5-chloro-1- oxoisoindolin-2-yl)piperidine-2,6- dione |
| 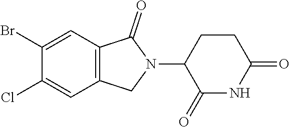 [SMILES: O=C1CCC(N2Cc3cc(Cl)c(Br)cc3C2=O)C(=O)N1] | 3-(6-bromo-5-chloro-1- oxoisoindolin-2-yl)piperidine-2,6- dione |
| 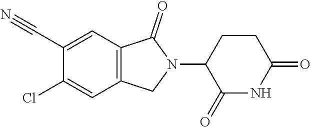 [SMILES: N#Cc1cc2c(cc1Cl)CN(C1CCC(=O)NC1=O)C2=O] | 6-chloro-2-(2,6-dioxopiperidin-3- yl)-3-oxoisoindoline-5-carbonitrile |
| 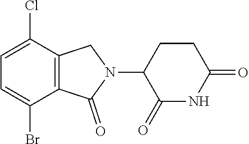 [SMILES: O=C1CCC(N2Cc3c(Cl)ccc(Br)c3C2=O)C(=O)N1] | 3-(7-bromo-4-chloro-1- oxoisoindolin-2-yl)piperidine-2,6- dione |
| 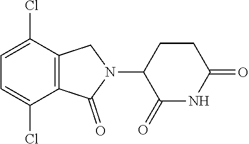 [SMILES: O=C1CCC(N2Cc3c(Cl)ccc(Cl)c3C2=O)C(=O)N1] | 3-(4,7-dichloro-1-oxoisoindolin-2- yl)piperidine-2,6-dione |
| 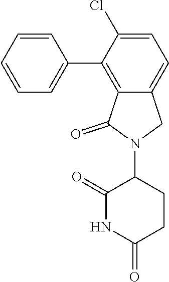 [SMILES: O=C1CCC(N2Cc3ccc(Cl)c(-c4ccccc4)c3C2=O)C(=O)N1] | 3-(6-chloro-1-oxo-7- phenylisoindolin-2-yl)piperidine- 2,6-dione |
| 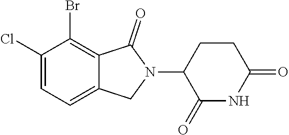 [SMILES: O=C1CCC(N2Cc3ccc(Cl)c(Br)c3C2=O)C(=O)N1] | 3-(7-bromo-6-chloro-1- oxoisoindolin-2-yl)piperidine-2,6- dione |
| 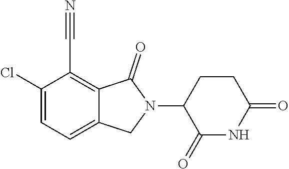 [SMILES: N#Cc1c(Cl)ccc2c1C(=O)N(C1CCC(=O)NC1=O)C2] | 5-chloro-2-(2,6-dioxopiperidin-3- yl)-3-oxoisoindoline-4-carbonitrile |
| 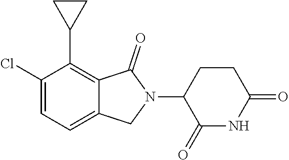 [SMILES: O=C1CCC(N2Cc3ccc(Cl)c(C4CC4)c3C2=O)C(=O)N1] | 3-(6-chloro-7-cyclopropyl-1- oxoisoindolin-2-yl)piperidine-2,6- dione |
| 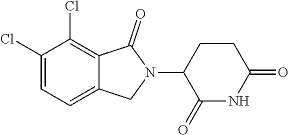 [SMILES: O=C1CCC(N2Cc3ccc(Cl)c(Cl)c3C2=O)C(=O)N1] | 3-(6,7-dichloro-1-oxoisoindolin-2- yl)piperidine-2,6-dione |
| 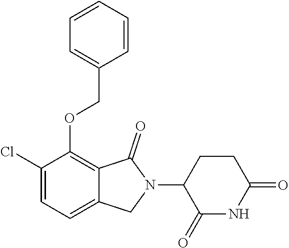 [SMILES: O=C1CCC(N2Cc3ccc(Cl)c(OCc4ccccc4)c3C2=O)C(=O)N1] | 3-(7-(benzyloxy)-6-chloro-1- oxoisoindolin-2-yl)piperidine-2,6-dione |
| 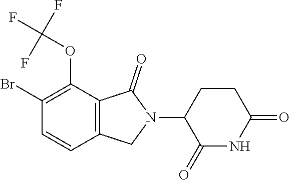 [SMILES: O=C1CCC(N2Cc3ccc(Br)c(OC(F)(F)F)c3C2=O)C(=O)N1] | 3-(6-bromo-1-oxo-7- (trifluoromethoxy)isoindolin-2- yl)piperidine-2,6-dione |
| 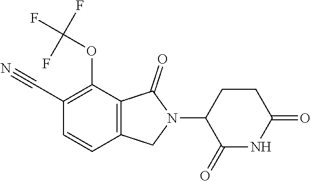 [SMILES: N#Cc1ccc2c(c1OC(F)(F)F)C(=O)N(C1CCC(=O)NC1=O)C2] | 2-(2,6-dioxopiperidin-3-yl)-3-oxo- 4-(trifluoromethoxy)isoindoline-5- carbonitrile |
| 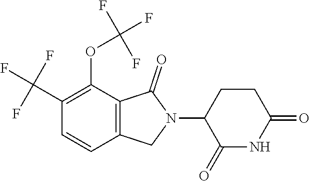 [SMILES: O=C1CCC(N2Cc3ccc(C(F)(F)F)c(OC(F)(F)F)c3C2=O)C(=O)N1] | 3-(1-oxo-7-(trifluoromethoxy)-6- (trifluoromethyl)isoindolin-2- yl)piperidine-2,6-dione |
| 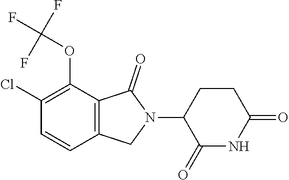 [SMILES: O=C1CCC(N2Cc3ccc(Cl)c(OC(F)(F)F)c3C2=O)C(=O)N1] | 3-(6-chloro-1-oxo-7- (trifluoromethoxy)isoindolin-2- yl)piperidine-2,6-dione |
| 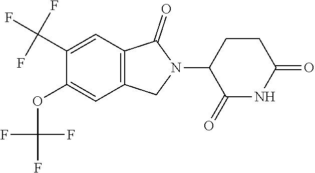 [SMILES: O=C1CCC(N2Cc3cc(OC(F)(F)F)c(C(F)(F)F)cc3C2=O)C(=O)N1] | 3-(1-oxo-5-(trifluoromethoxy)-6- (trifluoromethyl)isoindolin-2- yl)piperidine-2,6-dione |
| 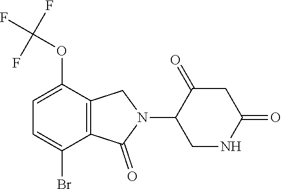 [SMILES: O=C1CC(=O)C(N2Cc3c(OC(F)(F)F)ccc(Br)c3C2=O)CN1] | 3-(7-bromo-1-oxo-4- (trifluoromethoxy)isoindolin-2- yl)piperidine-2,6-dione |
| 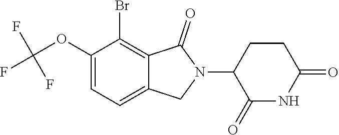 [SMILES: O=C1CCC(N2Cc3ccc(OC(F)(F)F)c(Br)c3C2=O)C(=O)N1] | 3-(7-bromo-1-oxo-6- (trifluoromethoxy)isoindolin-2- yl)piperidine-2,6-dione |
| 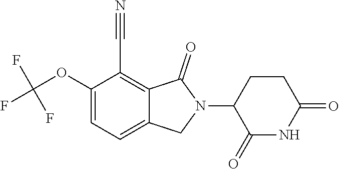 [SMILES: N#Cc1c(OC(F)(F)F)ccc2c1C(=O)N(C1CCC(=O)NC1=O)C2] | 2-(2,6-dioxopiperidin-3-yl)-3-oxo- 5-(trifluoromethoxy)isoindoline-4- carbonitrile |
| 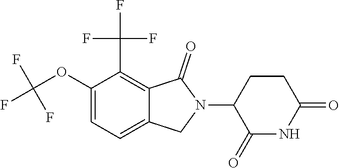 [SMILES: O=C1CCC(N2Cc3ccc(OC(F)(F)F)c(C(F)(F)F)c3C2=O)C(=O)N1] | 3-(1-oxo-6-(trifluoromethoxy)-7- (trifluoromethyl)isoindolin-2- yl)piperidine-2,6-dione |
| 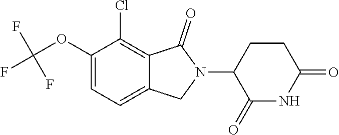 [SMILES: O=C1CCC(N2Cc3ccc(OC(F)(F)F)c(Cl)c3C2=O)C(=O)N1] | 3-(7-chloro-1-oxo-6- (trifluoromethoxy)isoindolin-2- yl)piperidine-2,6-dione |
| 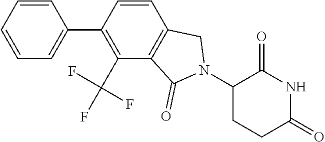 [SMILES: O=C1CCC(N2Cc3ccc(-c4ccccc4)c(C(F)(F)F)c3C2=O)C(=O)N1] | 3-(1-oxo-6-phenyl-7- (trifluoromethyl)isoindolin-2- yl)piperidine-2,6-dione |
| 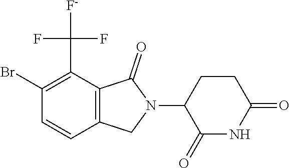 | 3-(6-bromo-1-oxo-7- (trifluoromethyl)isoindolin-2- yl)piperidine-2,6-dione |
| 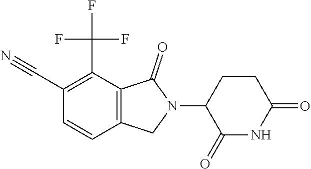 [SMILES: N#Cc1ccc2c(c1C(F)(F)F)C(=O)N(C1CCC(=O)NC1=O)C2] | 2-(2,6-dioxopiperidin-3-yl)-3-oxo- 4-(trifluoromethyl)isoindoline-5- carbonitrile |
| 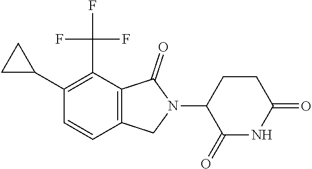 [SMILES: O=C1CCC(N2Cc3ccc(C4CC4)c(C(F)(F)F)c3C2=O)C(=O)N1] | 3-(6-cyclopropyl-1-oxo-7- (trifluoromethyl)isoindolin-2- yl)piperidine-2,6-dione |
| 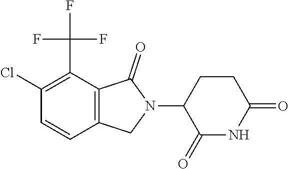 [SMILES: O=C1CCC(N2Cc3ccc(Cl)c(C(F)(F)F)c3C2=O)C(=O)N1] | 3-(6-chloro-1-oxo-7- (trifluoromethyl)isoindolin-2- yl)piperidine-2,6-dione |
| 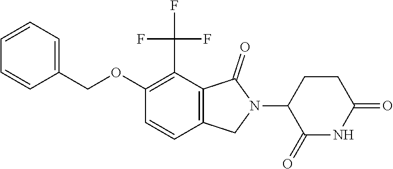 [SMILES: O=C1CCC(N2Cc3ccc(OCc4ccccc4)c(C(F)(F)F)c3C2=O)C(=O)N1] | 3-(6-benzyloxy)-1-oxo-7- (trifluoromethyl)isoindolin-2- yl)piperidine-2,6-dione |
| 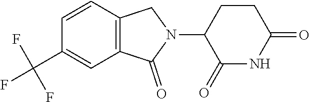 [SMILES: O=C1CCC(N2Cc3ccc(C(F)(F)F)cc3C2=O)C(=O)N1] | 3-(1-oxo-6- (trifluoromethyl)isoindolin-2- yl)piperidine-2,6-dione |
| 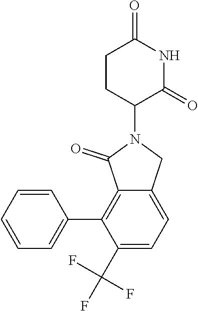 [SMILES: O=C1CCC(N2Cc3ccc(C(F)(F)F)c(-c4ccccc4)c3C2=O)C(=O)N1] | 3-(1-oxo-7-phenyl-6- (trifluoromethyl)isoindolin-2- yl)piperidine-2,6-dione |
| 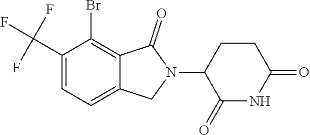 [SMILES: O=C1CCC(N2Cc3ccc(C(F)(F)F)c(Br)c3C2=O)C(=O)N1] | 3-(7-bromo-1-oxo-6- (trifluoromethyl)isoindolin-2- yl)piperidine-2,6-dione |
| 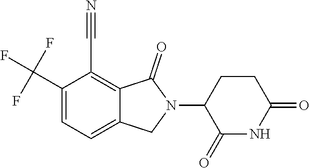 [SMILES: N#Cc1c(C(F)(F)F)ccc2c1C(=O)N(C1CCC(=O)NC1=O)C2] | 2-(2,6-dioxopiperidin-3-yl)-3-oxo- 5-(trifluoromethyl)isoindoline-4- carbonitrile |
| 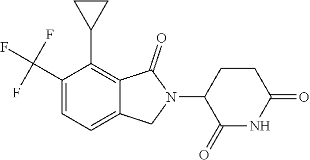 [SMILES: O=C1CCC(N2Cc3ccc(C(F)(F)F)c(C4CC4)c3C2=O)C(=O)N1] | 3-(7-cyclopropyl-1-oxo-6- (trifluoromethyl)isoindolin-2- yl)piperidine-2,6-dione |
| 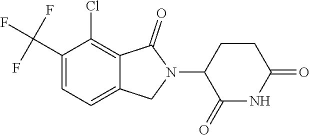 [SMILES: O=C1CCC(N2Cc3ccc(C(F)(F)F)c(Cl)c3C2=O)C(=O)N1] | 3-(7-chloro-1-oxo-6- (trifluoromethyl)isoindolin-2- yl)piperidine-2,6-dione |
| 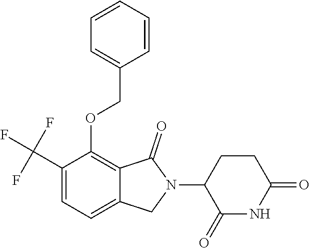 [SMILES: O=C1CCC(N2Cc3ccc(C(F)(F)F)c(OCc4ccccc4)c3C2=O)C(=O)N1] | 3-(7-(benzyloxy)-1-oxo-6- (trifluoromethyl)isoindolin-2- yl)piperidine-2,6-dione |
| 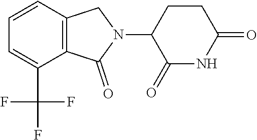 [SMILES: O=C1CCC(N2Cc3cccc(C(F)(F)F)c3C2=O)C(=O)N1] | 3-(1-oxo-7- (trifluoromethyl)isoindolin-2- yl)piperidine-2,6-dione |
| 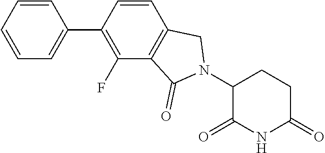 [SMILES: O=C1CCC(N2Cc3ccc(-c4ccccc4)c(F)c3C2=O)C(=O)N1] | 3-(7-fluoro-1-oxo-6- phenylisoindolin-2-yl)piperidine- 2,6-dione |
| 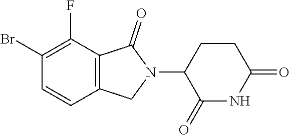 [SMILES: O=C1CCC(N2Cc3ccc(Br)c(F)c3C2=O)C(=O)N1] | 3-(6-bromo-7-fluoro-1- oxoisoindolin-2-yl)piperidine-2,6- dione |
| 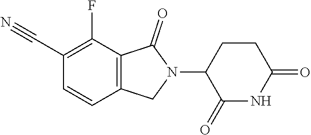 [SMILES: N#Cc1ccc2c(c1F)C(=O)N(C1CCC(=O)NC1=O)C2] | 2-(2,6-dioxopiperidin-3-yl)-4- fluoro-3-oxoisoindoline-5- carbonitrile |
| 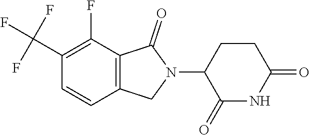 [SMILES: O=C1CCC(N2Cc3ccc(C(F)(F)F)c(F)c3C2=O)C(=O)N1] | 3-(7-fluoro-1-oxo-6- (trifluoromethyl)isoindolin-2- yl)piperidine-2,6-dione |
| 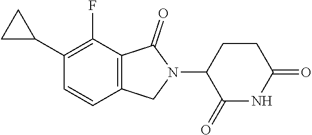 [SMILES: O=C1CCC(N2Cc3ccc(C4CC4)c(F)c3C2=O)C(=O)N1] | 3-(6-cyclopropyl-7-fluoro-1- oxoisoindolin-2-yl)piperidine-2,6- dione |
| 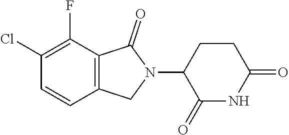 [SMILES: O=C1CCC(N2Cc3ccc(Cl)c(F)c3C2=O)C(=O)N1] | 3-(6-chloro-7-fluoro-1- oxoisoindolin-2-yl)piperidine-2,6- dione |
| 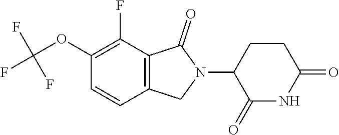 [SMILES: O=C1CCC(N2Cc3ccc(OC(F)(F)F)c(F)c3C2=O)C(=O)N1] | 3-(7-fluoro-1-oxo-6- (trifluoromethoxy)isoindolin-2- yl)piperidine-2,6-dione |
| 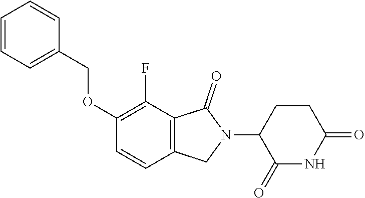 [SMILES: O=C1CCC(N2Cc3ccc(OCc4ccccc4)c(F)c3C2=O)C(=O)N1] | 3-(6-(benzyloxy)-7-fluoro-1- oxoisoindolin-2-yl)piperidine-2,6- dione |
| 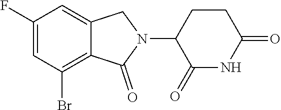 [SMILES: O=C1CCC(N2Cc3cc(F)cc(Br)c3C2=O)C(=O)N1] | 3-(7-bromo-5-fluoro-1- oxoisoindolin-2-yl)piperidine-2,6- dione |
| 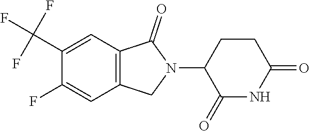 [SMILES: O=C1CCC(N2Cc3cc(F)c(C(F)(F)F)cc3C2=O)C(=O)N1] | 3-(5-fluoro-1-oxo-6- (trifluoromethyl)isoindolin-2- yl)piperidine-2,6-dione |
| 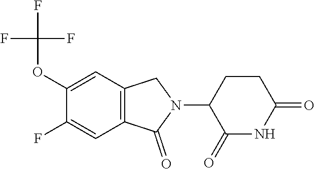 [SMILES: O=C1CCC(N2Cc3cc(OC(F)(F)F)c(F)cc3C2=O)C(=O)N1] | 3-(6-fluoro-1-oxo-5- (trifluoromethoxy)isoindolin-2- yl)piperidine-2,6-dione |
| 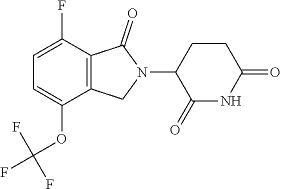 [SMILES: O=C1CCC(N2Cc3c(OC(F)(F)F)ccc(F)c3C2=O)C(=O)N1] | 3-(7-fluoro-1-oxo-4- (trifluoromethoxy)isoindolin-2- yl)pipieridine-2,6-dione |
| 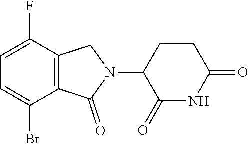 [SMILES: O=C1CCC(N2Cc3c(F)ccc(Br)c3C2=O)C(=O)N1] | 3-(7-bromo-4-fluoro-1- oxoisoindolin-2-yl)piperidine-2,6- dione |
| 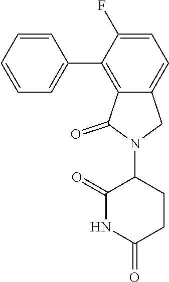 [SMILES: O=C1CCC(N2Cc3ccc(F)c(-c4ccccc4)c3C2=O)C(=O)N1] | 3-(6-fluoro-1-oxo-7- phenylisoindolin-2-yl)piperidine- 2,6-dione |
| 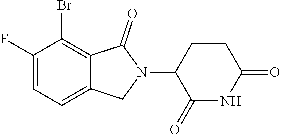 [SMILES: O=C1CCC(N2Cc3ccc(F)c(Br)c3C2=O)C(=O)N1] | 3-(7-bromo-6-fluoro-1- oxoisoindolin-2-yl)piperidine-2,6- dione |
| 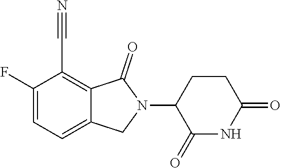 [SMILES: N#Cc1c(F)ccc2c1C(=O)N(C1CCC(=O)NC1=O)C2] | 2-(2,6-dioxopiperidin-3-yl)-5- fluoro-3-oxoisoindoline-4- carbonitrile |
| 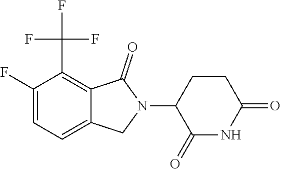 [SMILES: O=C1CCC(N2Cc3ccc(F)c(C(F)(F)F)c3C2=O)C(=O)N1] | 3-(6-fluoro-1-oxo-7- (trifluoromethyl)isoindolin-2- yl)piperidine-2,6-dione |
| 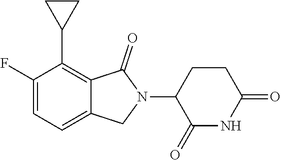 [SMILES: O=C1CCC(N2Cc3ccc(F)c(C4CC4)c3C2=O)C(=O)N1] | 3-(7-cyclopropyl-6-fluoro-1- oxoisoindolin-2-yl)piperidine-2,6- dione |
| 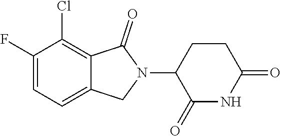 [SMILES: O=C1CCC(N2Cc3ccc(F)c(Cl)c3C2=O)C(=O)N1] | 3-(7-chloro-6-fluoro-1- oxoisoindolin-2-yl)piperidine-2,6- dione |
| 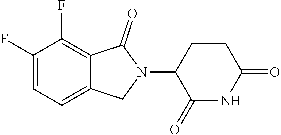 [SMILES: O=C1CCC(N2Cc3ccc(F)c(F)c3C2=O)C(=O)N1] | 3-(6,7-difluoro-1-oxoisoindolin-2- yl)piperidine-2,6-dione |
| 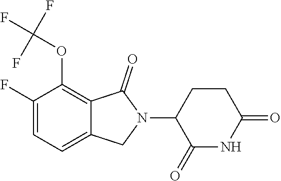 [SMILES: O=C1CCC(N2Cc3ccc(F)c(OC(F)(F)F)c3C2=O)C(=O)N1] | 3-(6-fluoro-1-oxo-7- (trifluoromethoxy)isoindolin-2- yl)piperidine-2,6-dione |
| 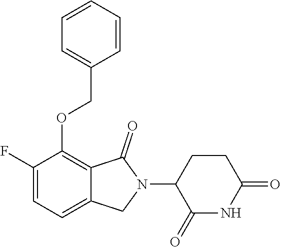 [SMILES: O=C1CCC(N2Cc3ccc(F)c(OCc4ccccc4)c3C2=O)C(=O)N1] | 3-(7-(benzyloxy)-6-fluoro-1- oxoisoindolin-2-yl)piperidine-2,6- dione |
| 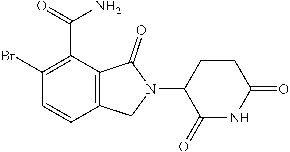 [SMILES: NC(=O)c1c(Br)ccc2c1C(=O)N(C1CCC(=O)NC1=O)C2] | 5-bromo-2-(2,6-dioxopiperidin-3- yl)-3-oxoisoindoline-4- carboxamide |
| 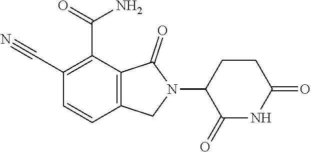 [SMILES: N#Cc1ccc2c(c1C(N)=O)C(=O)N(C1CCC(=O)NC1=O)C2] | 5-cyano-2-(2,6-dioxopiperidin-3- yl)-3-oxoisoindoline-4- carboxamide |
| 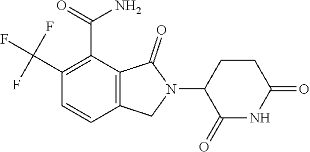 [SMILES: NC(=O)c1c(C(F)(F)F)ccc2c1C(=O)N(C1CCC(=O)NC1=O)C2] | 2-(2,6-dioxopiperidin-3-yl)-3-oxo- 5-(trifluoromethyl)isoindoline-4- carboxamide |
| 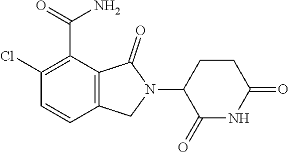 [SMILES: NC(=O)c1c(Cl)ccc2c1C(=O)N(C1CCC(=O)NC1=O)C2] | 5-chloro-2-(2,6-dioxopiperidin-3- yl)-3-oxoisoindoline-4- carboxamide |
| 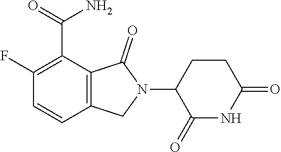 [SMILES: NC(=O)c1c(F)ccc2c1C(=O)N(C1CCC(=O)NC1=O)C2] | 2-(2,6-dioxopiperidin-3-yl)-5- fluoro-3-oxoisoindoline-4- carboxamide |
| 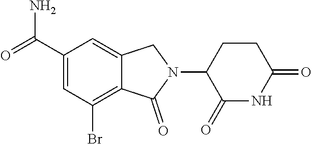 [SMILES: NC(=O)c1cc(Br)c2c(c1)CN(C1CCC(=O)NC1=O)C2=O] | 7-bromo-2-(2,6-dioxopiperidin-3- yl)-1-oxoisoindoline-5- carboxamide |
| 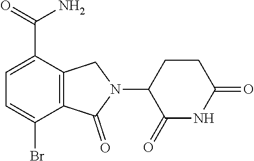 [SMILES: NC(=O)c1ccc(Br)c2c1CN(C1CCC(=O)NC1=O)C2=O] | 7-bromo-2-(2,6-dioxopiperidin-3- yl)-1-oxoisoindoline-4- carboxamide |
| 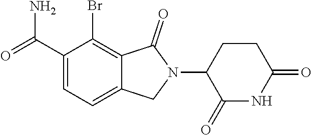 [SMILES: NC(=O)c1ccc2c(c1Br)C(=O)N(C1CCC(=O)NC1=O)C2] | 4-bromo-2-(2,6-dioxopiperidin-3- yl)-3-oxoisoindoline-5- carboxamide |
| 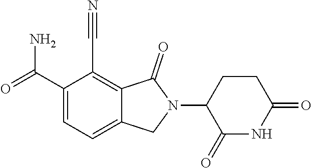 [SMILES: N#Cc1c(C(N)=O)ccc2c1C(=O)N(C1CCC(=O)NC1=O)C2] | 4-cyano-2-(2,6-dioxopiperidin-3- yl)-3-oxoisoindoline-5- carboxamide |
| 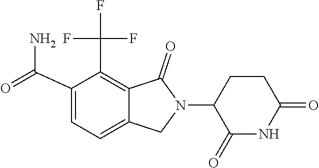 [SMILES: NC(=O)c1ccc2c(c1C(F)(F)F)C(=O)N(C1CCC(=O)NC1=O)C2] | 2-(2,6-dioxopiperidin-3-yl)-3-oxo- 4-(trifluoromethyl)isoindoline-5- carboxamide |
| 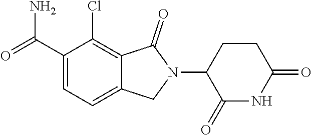 [SMILES: NC(=O)c1ccc2c(c1Cl)C(=O)N(C1CCC(=O)NC1=O)C2] | 4-chloro-2-(2,6-dioxopiperidin-3- yl)-3-oxoisoindoline-5- carboxamide |
| 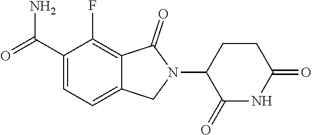 [SMILES: NC(=O)c1ccc2c(c1F)C(=O)N(C1CCC(=O)NC1=O)C2] | 2-(2,6-dioxopiperidin-3-yl)-4- fluoro-3-oxoisoindoline-5- carboxamide |
| 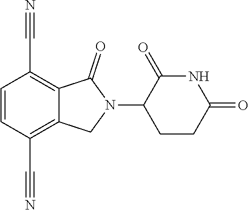 [SMILES: N#Cc1ccc(C#N)c2c1CN(C1CCC(=O)NC1=O)C2=O] | 2-(2,6-dioxopiperidin-3-yl)-1- oxoisoindoline-4,7-dicarbonitrile |
| 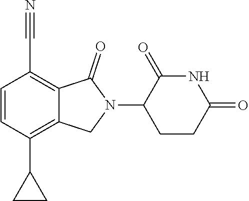 [SMILES: N#Cc1ccc(C2CC2)c2c1C(=O)N(C1CCC(=O)NC1=O)C2] | 7-cyclopropyl-2-(2,6- dioxopiperidin-3-yl)-3- oxoisoindoline-4-carbonitrile |
| 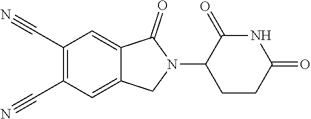 [SMILES: N#Cc1cc2c(cc1C#N)C(=O)N(C1CCC(=O)NC1=O)C2] | 2-(2,6-dioxopiperidin-3-yl)-1- oxoisoindoline-5,6-dicarbonitrile |
| 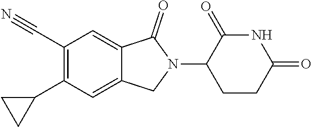 [SMILES: N#Cc1cc2c(cc1C1CC1)CN(C1CCC(=O)NC1=O)C2=O] | 6-cyclopropyl-2-(2,6- dioxopiperidin-3-yl)-3- oxoisoindoline-5-carbonitrile |
| 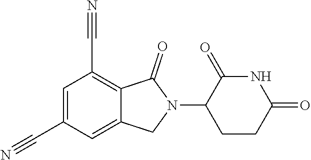 [SMILES: N#Cc1cc(C#N)c2c(c1)CN(C1CCC(=O)NC1=O)C2=O] | 2-(2,6-dioxopiperidin-3-yl)-3- oxoisoindoline-4,6-dicarbonitrile |
| 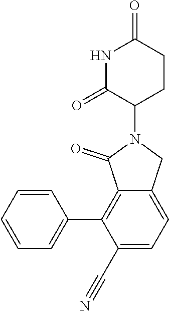 [SMILES: N#Cc1ccc2c(c1-c1ccccc1)C(=O)N(C1CCC(=O)NC1=O)C2] | 2-(2,6-dioxopiperidin-3-yl)-3-oxo- 4-phenylisoindoline-5-carbonitrile |
| 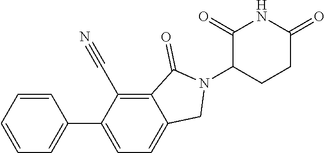 [SMILES: N#Cc1c(-c2ccccc2)ccc2c1C(=O)N(C1CCC(=O)NC1=O)C2] | 2-(2,6-dioxopiperidin-3-yl)-3-oxo- 5-phenylisoindoline-4-carbonitrile |
| 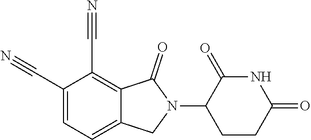 [SMILES: N#Cc1ccc2c(c1C#N)C(=O)N(C1CCC(=O)NC1=O)C2] | 2-(2,6-dioxopiperidin-3-yl)-3- oxoisoindoline-4,5-dicarbonitrile |
| 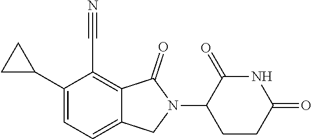 [SMILES: N#Cc1c(C2CC2)ccc2c1C(=O)N(C1CCC(=O)NC1=O)C2] | 5-cyclopropyl-2-(2,6- dioxopiperidin-3-yl)-3- oxoisoindoline-4-carbonitrile |
| 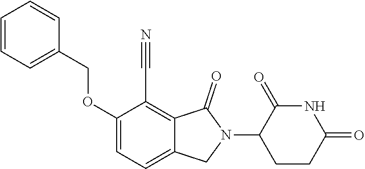 [SMILES: N#Cc1c(OCc2ccccc2)ccc2c1C(=O)N(C1CCC(=O)NC1=O)C2] | 5-(benzyloxy)-2-(2,6- dioxopiperidin-3-yl)-3- oxoisoindoline-4-carbonitrile |
| 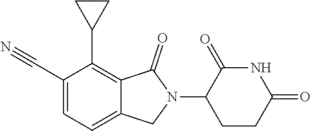 [SMILES: N#Cc1ccc2c(c1C1CC1)C(=O)N(C1CCC(=O)NC1=O)C2] | 4-cyclopropyl-2-(2,6- dioxopiperidin-3-yl)-3- oxoisoindoline-5-carbonitrile |
| 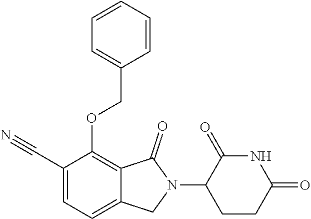 [SMILES: N#Cc1ccc2c(c1OCc1ccccc1)C(=O)N(C1CCC(=O)NC1=O)C2] | 4-(benzyloxy)-2-(2,6- dioxopiperidin-3-yl)-3- oxoisoindoline-5-carbonitrile |
| 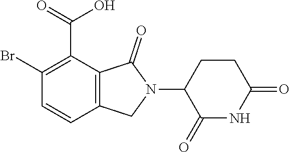 [SMILES: O=C1CCC(N2Cc3ccc(Br)c(C(=O)O)c3C2=O)C(=O)N1] | 5-bromo-2-(2,6-dioxopiperidin-3- yl)-3-oxoisoindoline-4-carboxylic acid |
| 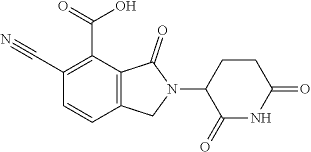 [SMILES: N#Cc1ccc2c(c1C(=O)O)C(=O)N(C1CCC(=O)NC1=O)C2] | 5-cyano-2-(2,6-dioxopiperidin-3- yl)-3-oxoisoindoline-4-carboxylic acid |
| 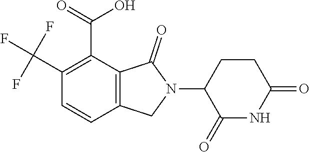 [SMILES: O=C1CCC(N2Cc3ccc(C(F)(F)F)c(C(=O)O)c3C2=O)C(=O)N1] | 2-(2,6-dioxopiperidin-3-yl)-3-oxo- 5-(trifluoromethyl)isoindoline-4- carboxylic acid |
| 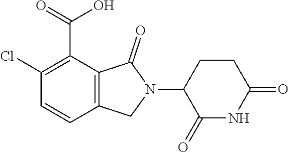 [SMILES: O=C1CCC(N2Cc3ccc(Cl)c(C(=O)O)c3C2=O)C(=O)N1] | 5-chloro-2-(2,6-dioxopiperidin-3- yl)-3-oxoisoindoline-4-carboxylic acid |
| 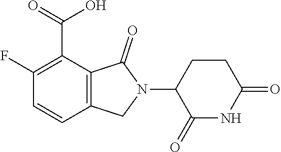 [SMILES: O=C1CCC(N2Cc3ccc(F)c(C(=O)O)c3C2=O)C(=O)N1] | 2-(2,6-dioxopiperidin-3-yl)-5- fluoro-3-oxoisoindoline-4- carboxylic acid |
| 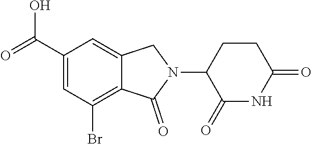 [SMILES: O=C1CCC(N2Cc3cc(C(=O)O)cc(Br)c3C2=O)C(=O)N1] | 7-bromo-2-(2,6-dioxopiperidin-3- yl)-1-oxoisoindoline-5-carboxylic acid |
| 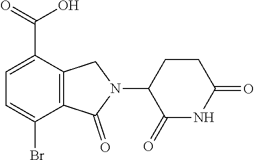 [SMILES: O=C1CCC(N2Cc3c(C(=O)O)ccc(Br)c3C2=O)C(=O)N1] | 7-bromo-2-(2,6-dioxopiperidin-3- yl)-1-oxoisoindoline-4-carboxylic acid |
| 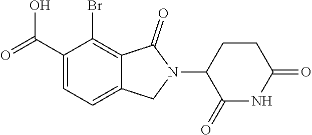 [SMILES: O=C1CCC(N2Cc3ccc(C(=O)O)c(Br)c3C2=O)C(=O)N1] | 4-bromo-2-(2,6-dioxopiperidin-3- yl)-3-oxoisoindoline-5-carboxylic acid |
| 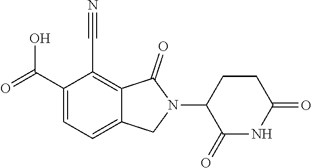 [SMILES: N#Cc1c(C(=O)O)ccc2c1C(=O)N(C1CCC(=O)NC1=O)C2] | 4-cyano-2-(2,6-dioxopiperidin-3- yl)-3-oxoisoindoline-5-carboxylic acid |
| 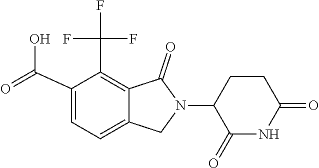 [SMILES: O=C1CCC(N2Cc3ccc(C(=O)O)c(C(F)(F)F)c3C2=O)C(=O)N1] | 2-(2,6-dioxopiperidin-3-yl)-3-oxo- 4-(trifluoromethyl)isoindoline-5- carboxylic acid |
| 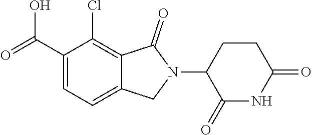 [SMILES: O=C1CCC(N2Cc3ccc(C(=O)O)c(Cl)c3C2=O)C(=O)N1] | 4-chloro-2-(2,6-dioxopiperidin-3- yl)-3-oxoisoindoline-5-carboxylic acid |
| 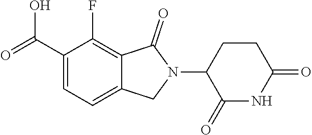 [SMILES: O=C1CCC(N2Cc3ccc(C(=O)O)c(F)c3C2=O)C(=O)N1] | 2-(2,6-dioxopiperidin-3-yl)-4- fluoro-3-oxoisoindoline-5- carboxylic acid |

Cereblon is a eukaryotic protein ranging from 400-600 residues in length. The human cereblon (SEQ ID NO: 1) is about 442 residues in length, and is encoded by the CRBN gene. The cereblon protein comprises a central LON domain (residues 80-317) followed by a C-terminal CULT domain. The LON domain is further subdivided into an N-terminal LON-N subdomain, a four helix bundle, and a C-terminal LON-C subdomain.

Further Forms of Compounds

In one aspect, the compounds in Tables 1-5 possesses one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis, trans, syn, anti, entgegen (E), and zusammen (Z) isomers as well as the appropriate mixtures thereof. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In one aspect, stereoisomers are obtained by stereoselective synthesis.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

Compounds described herein may be formed as, and/or used as, acceptable salts. The type of acceptable salts, include, but are not limited to: (1) acid addition salts, formed by reacting the free base form of the compound with an acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms, particularly solvates. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

Synthesis of Compounds

In some embodiments, the synthesis of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, Fisher Scientific (Fisher Chemicals), and Acros Organics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein. As a guide the following synthetic methods may be utilized.

In the reactions described, it may be necessary to protect reactive functional groups, for example hydroxy, amino, imino, thio or carboxy groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure).

In some embodiments, the compounds of Formula (I), Formula (II), Formula (IIA), and Formula (IIB) are purchased from a variety of vendors, including Sigma Aldrich, Acros, Fisher, Fluka, Santa Cruz, CombiBlocks, BioBlocks, and Matrix Scientific.

Cells, Analytical Techniques, and Instrumentation

In certain embodiments, also described herein are methods for profiling cereblon to determine a reactive or ligandable cysteine residue. In some instances, the methods comprising profiling a cereblon cell sample or a cereblon cell lysate sample. In some embodiments, the cell sample or cell lysate sample is obtained from cells of an animal. In some instances, the animal cell includes a cell from a marine invertebrate, fish, insects, amphibian, reptile, or mammal. In some instances, the mammalian cell is a primate, ape, equine, bovine, porcine, canine, feline, or rodent. In some instances, the mammal is a primate, ape, dog, cat, rabbit, ferret, or the like. In some cases, the rodent is a mouse, rat, hamster, gerbil, hamster, chinchilla, or guinea pig. In some embodiments, the bird cell is from a canary, parakeet or parrots. In some embodiments, the reptile cell is from a turtles, lizard or snake. In some cases, the fish cell is from a tropical fish. In some cases, the fish cell is from a zebrafish (e.g. Danino rerio). In some cases, the worm cell is from a nematode (e.g. C. elegans). In some cases, the amphibian cell is from a frog. In some embodiments, the arthropod cell is from a tarantula or hermit crab.

In some embodiments, the cereblon cell sample or cell lysate sample is obtained from a mammalian cell. In some instances, the mammalian cell is an epithelial cell, connective tissue cell, hormone secreting cell, a nerve cell, a skeletal muscle cell, a blood cell, or an immune system cell.

Exemplary mammalian cells include, but are not limited to, 293A cell line, 293FT cell line, 293F cells, 293 H cells, HEK 293 cells, CHO DG44 cells, CHO—S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO—S cells, GripTite™ 293 MSR cell line, GS-CHO cell line, HepaRG™ cells, T-REx™ Jurkat cell line, Per.C6 cells, T-REx™-293 cell line, T-REx™-CHO cell line, T-REx™-HeLa cell line, NC-HIMT cell line, and PC12 cell line.

In some instances, the cereblon cell sample or cell lysate sample is obtained from cells of a tumor cell line. In some instances, the cell sample or cell lysate sample is obtained from cells of a solid tumor cell line. In some instances, the solid tumor cell line is a sarcoma cell line. In some instances, the solid tumor cell line is a carcinoma cell line. In some embodiments, the sarcoma cell line is obtained from a cell line of alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, telangiectatic osteosarcoma.

In some embodiments, the carcinoma cell line is obtained from a cell line of adenocarcinoma, squamous cell carcinoma, adenosquamous carcinoma, anaplastic carcinoma, large cell carcinoma, small cell carcinoma, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

In some instances, the cereblon cell sample or cell lysate sample is obtained from cells of a hematologic malignant cell line. In some instances, the hematologic malignant cell line is a T-cell cell line. In some instances, B-cell cell line. In some instances, the hematologic malignant cell line is obtained from a T-cell cell line of: peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some instances, the hematologic malignant cell line is obtained from a B-cell cell line of: acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, the cereblon cell sample or cell lysate sample is obtained from a tumor cell line. Exemplary tumor cell line includes, but is not limited to, 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino. KYSE or other cell lines expressing SALL4.

In some embodiments, the cereblon cell sample or cell lysate sample is from any tissue or fluid from an individual. Samples include, but are not limited to, tissue (e.g. connective tissue, muscle tissue, nervous tissue, or epithelial tissue), whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In some embodiments, the cell sample or cell lysate sample is a tissue sample, such as a sample obtained from a biopsy or a tumor tissue sample. In some embodiments, the cell sample or cell lysate sample is a blood serum sample. In some embodiments, the cell sample or cell lysate sample is a blood cell sample containing one or more peripheral blood mononuclear cells (PBMCs). In some embodiments, the cell sample or cell lysate sample contains one or more circulating tumor cells (CTCs). In some embodiments, the cell sample or cell lysate sample contains one or more disseminated tumor cells (DTC, e.g., in a bone marrow aspirate sample).

In some embodiments, the cereblon cell sample or cell lysate sample is obtained from the individual by any suitable means of obtaining the sample using well-known and routine clinical methods. Procedures for obtaining tissue samples from an individual are well known. For example, procedures for drawing and processing tissue sample such as from a needle aspiration biopsy is well-known and is employed to obtain a sample for use in the methods provided. Typically, for collection of such a tissue sample, a thin hollow needle is inserted into a mass such as a tumor mass for sampling of cells that, after being stained, will be examined under a microscope.

Sample Preparation and Analysis

In some embodiments, a cereblon sample solution comprises a cell sample, a cell lysate sample, or a sample comprising isolated proteins. In some instances, the sample solution comprises a solution such as a buffer (e.g. phosphate buffered saline) or a media. In some embodiments, the media is an isotopically labeled media. In some instances, the sample solution is a cell solution.

In some embodiments, the cereblon solution sample (e.g., cell sample, cell lysate sample, or comprising isolated proteins) is incubated with a compound of Formula (I) for analysis of protein-compound interactions. In some instances, the solution sample (e.g., cell sample, cell lysate sample, or comprising isolated proteins) is further incubated in the presence of an additional compound prior to addition of the compound of Formula (I). In other instances, the solution sample (e.g., cell sample, cell lysate sample, or comprising isolated proteins) is further incubated with a ligand, in which the ligand does not contain a photoreactive moiety and/or an alkyne group. In such instances, the solution sample is incubated with a compound and a ligand for competitive protein profiling analysis.

In some cases, the cereblon cell sample or the cell lysate sample is compared with a control. In some cases, a difference is observed between a set of compound protein interactions between the sample and the control. In some instances, the difference correlates to the interaction between the small molecule fragment and the proteins.

In some embodiments, one or more methods are utilized for labeling a cereblon solution sample (e.g. cell sample, cell lysate sample, or comprising isolated proteins) for analysis of compound protein interactions. In some instances, a method comprises labeling the sample (e.g. cell sample, cell lysate sample, or comprising isolated proteins) with an enriched media. In some cases, the sample (e.g. cell sample, cell lysate sample, or comprising isolated proteins) is labeled with isotope-labeled amino acids, such as ¹⁵C or ¹⁵N-labeled amino acids. In some cases, the labeled sample is further compared with a non-labeled sample to detect differences in compound protein interactions between the two samples. In some instances, this difference is a difference of a target protein and its interaction with a small molecule ligand in the labeled sample versus the non-labeled sample. In some instances, the difference is an increase, decrease or a lack of protein-compound interaction in the two samples. In some instances, the isotope-labeled method is termed SILAC, stable isotope labeling using amino acids in cell culture.

In some embodiments, a method comprises incubating a solution sample (e.g. cell sample, cell lysate sample, or comprising isolated proteins) with a labeling group (e.g., an isotopically labeled labeling group) to tag one or more proteins of interest for further analysis. In such cases, the labeling group comprises a biotin, a streptavidin, bead, resin, a solid support, or a combination thereof, and further comprises a linker that is optionally isotopically labeled. As described above, the linker can be about 5, 6, 7, 8, 9, 10, 11, 12, 13, 14, 15 or more residues in length and might further comprise a cleavage site, such as a protease cleavage site (e.g., TEV cleavage site). In some cases, the labeling group is a biotin-linker moiety, which is optionally isotopically labeled with ¹³C and ¹⁵N atoms at one or more amino acid residue positions within the linker. In some cases, the biotin-linker moiety is a isotopically-labeled TEV-tag as described in Weerapana, et al., “Quantitative reactivity profiling predicts functional cysteines in proteomes,” Nature 468(7325): 790-795.

In some embodiments, an isotopic reductive dimethylation (ReDi) method is utilized for processing a sample. In some cases, the ReDi labeling method involves reacting peptides with formaldehyde to form a Schiff base, which is then reduced by cyanoborohydride. This reaction dimethylates free amino groups on N-termini and lysine side chains and monomethylates N-terminal prolines. In some cases, the ReDi labeling method comprises methylating peptides from a first processed sample with a “light” label using reagents with hydrogen atoms in their natural isotopic distribution and peptides from a second processed sample with a “heavy” label using deuterated formaldehyde and cyanoborohydride. Subsequent proteomic analysis (e.g., mass spectrometry analysis) based on a relative peptide abundance between the heavy and light peptide version might be used for analysis of compound-protein interactions.

In some embodiments, isobaric tags for relative and absolute quantitation (iTRAQ) method is utilized for processing a sample. In some cases, the iTRAQ method is based on the covalent labeling of the N-terminus and side chain amines of peptides from a processed sample. In some cases, reagent such as 4-plex or 8-plex is used for labeling the peptides.

In some embodiments, the compound-protein complex is further conjugated to a chromophore, such as a fluorophore. In some instances, the compound-protein complex is separated and visualized utilizing an electrophoresis system, such as through a gel electrophoresis, or a capillary electrophoresis. Exemplary gel electrophoresis includes agarose based gels, polyacrylamide based gels, or starch based gels. In some instances, the compound-protein is subjected to a native electrophoresis condition. In some instances, the compound-protein is subjected to a denaturing electrophoresis condition.

In some instances, the compound-protein after harvesting is further fragmentized to generate protein fragments. In some instances, fragmentation is generated through mechanical stress, pressure, or chemical means. In some instances, the protein from the compound-protein complexes is fragmented by a chemical means. In some embodiments, the chemical means is a protease. Exemplary proteases include, but are not limited to, serine proteases such as chymotrypsin A, penicillin G acylase precursor, dipeptidase E, DmpA aminopeptidase, subtilisin, prolyl oligopeptidase, D-Ala-D-Ala peptidase C, signal peptidase I, cytomegalovirus assemblin, Lon-A peptidase, peptidase Clp, Escherichia coli phage K1F endosialidase CIMCD self-cleaving protein, nucleoporin 145, lactoferrin, murein tetrapeptidase LD-carboxypeptidase, or rhomboid-1; threonine proteases such as ornithine acetyltransferase; cysteine proteases such as TEV protease, amidophosphoribosyltransferase precursor, gamma-glutamyl hydrolase (Rattus norvegicus), hedgehog protein, DmpA aminopeptidase, papain, bromelain, cathepsin K, calpain, caspase-1, separase, adenain, pyroglutamyl-peptidase I, sortase A, hepatitis C virus peptidase 2, sindbis virus-type nsP2 peptidase, dipeptidyl-peptidase VI, or DeSI-1 peptidase; aspartate proteases such as beta-secretase 1 (BACE1), beta-secretase 2 (BACE2), cathepsin D, cathepsin E, chymosin, napsin-A, nepenthesin, pepsin, plasmepsin, presenilin, or renin; glutamic acid proteases such as AfuGprA; and metalloproteases such as peptidase_M48.

In some instances, the fragmentation is a random fragmentation. In some instances, the fragmentation generates specific lengths of protein fragments, or the shearing occurs at particular sequence of amino acid regions.

In some instances, the protein fragments are further analyzed by a proteomic method such as by liquid chromatography (LC) (e.g. high performance liquid chromatography), liquid chromatography-mass spectrometry (LC-MS), matrix-assisted laser desorption/ionization (MALDI-TOF), gas chromatography-mass spectrometry (GC-MS), capillary electrophoresis-mass spectrometry (CE-MS), or nuclear magnetic resonance imaging (NMR).

In some embodiments, the LC method is any suitable LC methods well known in the art, for separation of a sample into its individual parts. This separation occurs based on the interaction of the sample with the mobile and stationary phases. Since there are many stationary/mobile phase combinations that are employed when separating a mixture, there are several different types of chromatography that are classified based on the physical states of those phases. In some embodiments, the LC is further classified as normal-phase chromatography, reverse-phase chromatography, size-exclusion chromatography, ion-exchange chromatography, affinity chromatography, displacement chromatography, partition chromatography, flash chromatography, chiral chromatography, and aqueous normal-phase chromatography.

In some embodiments, the LC method is a high performance liquid chromatography (HPLC) method. In some embodiments, the HPLC method is further categorized as normal-phase chromatography, reverse-phase chromatography, size-exclusion chromatography, ion-exchange chromatography, affinity chromatography, displacement chromatography, partition chromatography, chiral chromatography, and aqueous normal-phase chromatography.

In some embodiments, the HPLC method of the present disclosure is performed by any standard techniques well known in the art. Exemplary HPLC methods include hydrophilic interaction liquid chromatography (HILIC), electrostatic repulsion-hydrophilic interaction liquid chromatography (ERLIC) and reverse phase liquid chromatography (RPLC).

In some embodiments, the LC is coupled to a mass spectroscopy as a LC-MS method. In some embodiments, the LC-MS method includes ultra-performance liquid chromatography-electrospray ionization quadrupole time-of-flight mass spectrometry (UPLC-ESI-QTOF-MS), ultra-performance liquid chromatography-electrospray ionization tandem mass spectrometry (UPLC-ESI-MS/MS), reverse phase liquid chromatography-mass spectrometry (RPLC-MS), hydrophilic interaction liquid chromatography-mass spectrometry (HILIC-MS), hydrophilic interaction liquid chromatography-triple quadrupole tandem mass spectrometry (HILIC-QQQ), electrostatic repulsion-hydrophilic interaction liquid chromatography-mass spectrometry (ERLIC-MS), liquid chromatography time-of-flight mass spectrometry (LC-QTOF-MS), liquid chromatography-tandem mass spectrometry (LC-MS/MS), multidimensional liquid chromatography coupled with tandem mass spectrometry (LC/LC-MS/MS). In some instances, the LC-MS method is LC/LC-MS/MS. In some embodiments, the LC-MS methods of the present disclosure are performed by standard techniques well known in the art.

In some embodiments, the GC is coupled to a mass spectroscopy as a GC-MS method. In some embodiments, the GC-MS method includes two-dimensional gas chromatography time-of-flight mass spectrometry (GC*GC-TOFMS), gas chromatography time-of-flight mass spectrometry (GC-QTOF-MS) and gas chromatography-tandem mass spectrometry (GC-MS/MS).

In some embodiments, CE is coupled to a mass spectroscopy as a CE-MS method. In some embodiments, the CE-MS method includes capillary electrophoresis-negative electrospray ionization-mass spectrometry (CE-ESI-MS), capillary electrophoresis-negative electrospray ionization-quadrupole time of flight-mass spectrometry (CE-ESI-QTOF-MS) and capillary electrophoresis-quadrupole time of flight-mass spectrometry (CE-QTOF-MS).

In some embodiments, the nuclear magnetic resonance (NMR) method is any suitable method well known in the art for the detection of one or more cysteine binding proteins or protein fragments disclosed herein. In some embodiments, the NMR method includes one dimensional (1D) NMR methods, two dimensional (2D) NMR methods, solid state NMR methods and NMR chromatography. Exemplary 1D NMR methods include ¹Hydrogen, ¹³Carbon, ¹⁵Nitrogen, ¹⁷Oxygen, ¹⁹Fluorine, ³¹Phosphorus, ³⁹Potassium, ²³Sodium, ³³Sulfur, ⁸⁷Strontium, ²⁷Aluminium, ⁴³Calcium, ³⁵Chlorine, ³⁷Chlorine, ⁶³Copper, ⁶⁵Copper, ⁵⁷Iron, ²⁵Magnesium, ¹⁹⁹Mercury or ⁶⁷Zinc NMR method, distortionless enhancement by polarization transfer (DEPT) method, attached proton test (APT) method and 1D-incredible natural abundance double quantum transition experiment (INADEQUATE) method. Exemplary 2D NMR methods include correlation spectroscopy (COSY), total correlation spectroscopy (TOCSY), 2D-INADEQUATE, 2D-adequate double quantum transfer experiment (ADEQUATE), nuclear overhauser effect spectroscopy (NOSEY), rotating-frame NOE spectroscopy (ROESY), heteronuclear multiple-quantum correlation spectroscopy (HMQC), heteronuclear single quantum coherence spectroscopy (HSQC), short range coupling and long range coupling methods. Exemplary solid state NMR method include solid state ¹³Carbon NMR, high resolution magic angle spinning (HR-MAS) and cross polarization magic angle spinning (CP-MAS) NMR methods. Exemplary NMR techniques include diffusion ordered spectroscopy (DOSY), DOSY-TOCSY and DOSY-HSQC.

In some embodiments, the protein fragments are analyzed by method as described in Weerapana et al., “Quantitative reactivity profiling predicts functional cysteines in proteomes,” Nature, 468:790-795 (2010).

In some embodiments, the results from the mass spectroscopy method are analyzed by an algorithm for protein identification. In some embodiments, the algorithm combines the results from the mass spectroscopy method with a protein sequence database for protein identification. In some embodiments, the algorithm comprises ProLuCID algorithm, Probity, Scaffold, SEQUEST, or Mascot.

In some embodiments, a value is assigned to each of the protein from the compound-protein complex. In some embodiments, the value assigned to each of the protein from the compound-protein complex is obtained from the mass spectroscopy analysis. In some instances, the value is the area-under-the curve from a plot of signal intensity as a function of mass-to-charge ratio. In some instances, the value correlates with the reactivity of a Lys residue within a protein.

In some instances, a ratio between a first value obtained from a first protein sample and a second value obtained from a second protein sample is calculated. In some instances, the ratio is greater than 2.5, 3, 3.5, 4, 4.5, 5, 6, 7, 8, 9, 10, 11, 12, 13, 14, 15, 16, 17, 18, 19, or 20. In some cases, the ratio is at most 20.

In some instances, the ratio is calculated based on averaged values. In some instances, the averaged value is an average of at least two, three, or four values of the protein from each cell solution, or that the protein is observed at least two, three, or four times in each cell solution and a value is assigned to each observed time. In some instances, the ratio further has a standard deviation of less than 12, 10, or 8.

In some instances, a value is not an averaged value. In some instances, the ratio is calculated based on value of a protein observed only once in a cell population. In some instances, the ratio is assigned with a value of 20.

Kits/Article of Manufacture

Disclosed herein, in certain embodiments, are kits and articles of manufacture for use to generate a cereblon-compound adduct or with one or more methods described herein. In some embodiments, described herein is a kit for detecting cereblon ligand interaction. In some embodiments, such kit includes small molecule ligands described herein, small molecule fragments or libraries, compounds described herein, and/or controls, and reagents suitable for carrying out one or more of the methods described herein. In some instances, the kit further comprises samples, such as a cell sample, and suitable solutions such as buffers or media. In some embodiments, the kit further comprises recombinant cereblon protein for use in one or more of the methods described herein. In some embodiments, additional components of the kit comprises a carrier, package, or container that is compartmentalized to receive one or more containers such as vials, tubes, and the like, each of the container(s) comprising one of the separate elements to be used in a method described herein. Suitable containers include, for example, bottles, vials, plates, syringes, and test tubes. In one embodiment, the containers are formed from a variety of materials such as glass or plastic.

The articles of manufacture provided herein contain packaging materials. Examples of pharmaceutical packaging materials include, but are not limited to, bottles, tubes, bags, containers, and any packaging material suitable for a selected formulation and intended mode of use.

For example, the container(s) include test compounds and one or more reagents for use in a method disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

“Alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (i-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

“Alkoxy” refers to a radical of the formula —OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy.

“Heteroalkylene” refers to an alkyl radical as described above where one or more carbon atoms of the alkyl is replaced with a O, N or S atom. “Heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkyl groups include, but are not limited to —OCH₂OMe, —OCH₂CH₂OMe, or —OCH₂CH₂OCH₂CH₂NH₂. Representative heteroalkylene groups include, but are not limited to —OCH₂CH₂O—, —OCH₂CH₂OCH₂CH₂O—, or —OCH₂CH₂OCH₂CH₂OCH₂CH₂O—.

“Alkylamino” refers to a radical of the formula —NHR or —NRR where each R is, independently, an alkyl radical as defined above. Unless stated otherwise specifically in the specification, an alkylamino group may be optionally substituted as described below.

The term “aromatic” refers to a planar ring having a delocalized n-electron system containing 4n+2 π electrons, where n is an integer. Aromatics can be optionally substituted. The term “aromatic” includes both aryl groups (e.g., phenyl, naphthalenyl) and heteroaryl groups (e.g., pyridinyl, quinolinyl).

“Aryl” refers to an aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted.

“Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to:

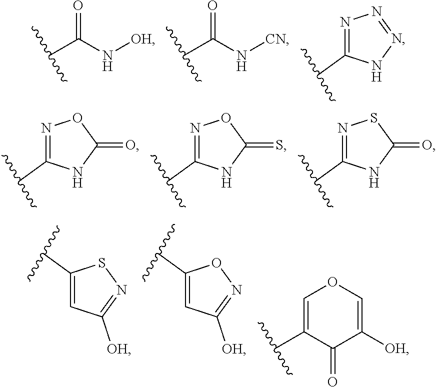

and the like.

“Cycloalkyl” refers to a monocyclic or polycyclic non-aromatic radical, wherein each of the atoms forming the ring (i.e. skeletal atoms) is a carbon atom. Cycloalkyls may be saturated, or partially unsaturated. Cycloalkyls may be fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cyclcoalkyl radicals include, for example, cyclopropyl, cyclobutyl, cyclopentyl, cyclohexyl, cycloheptyl, and cyclooctyl. In some embodiments, the monocyclic cyclcoalkyl is cyclopropyl, cyclobutyl, cyclopentyl or cyclohexyl. In some embodiments, the monocyclic cyclcoalkyl is cyclopentyl. Polycyclic radicals include, for example, adamantyl, norbornyl, decalinyl, and 3,4-dihydronaphthalen-1(2H)-one. Unless otherwise stated specifically in the specification, a cycloalkyl group may be optionally substituted.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” refers to an alkoxy radical, as defined above, that is substituted by one or more halo radicals, as defined above, e.g., trifluoromethoxy, difluoromethoxy, fluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

“Heterocycloalkyl” or “heterocyclyl” or “heterocyclic ring” refers to a stable 3- to 14-membered non-aromatic ring radical comprising 2 to 10 carbon atoms and from one to 4 heteroatoms selected from the group consisting of nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 10 carbons in the ring. In some embodiments, heterocycloalkyls have from 2 to 8 carbons in the ring. In some embodiments, heterocycloalkyls have from 2 to 8 carbons in the ring and 1 or 2 N atoms. In some embodiments, heterocycloalkyls have from 2 to 10 carbons, 0-2 N atoms, 0-2 O atoms, and 0-1 S atoms in the ring. In some embodiments, heterocycloalkyls have from 2 to 10 carbons, 1-2 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

“Heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, a bicyclic heteroaryl is a C₆-C₉heteroaryl.

The term “optionally substituted” or “substituted” means that the referenced group may be substituted with one or more additional group(s) individually and independently selected from alkyl, haloalkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆alkylalkyne, halogen, acyl, acyloxy, —CO₂H, —CO₂alkyl, nitro, and amino, including mono- and di-substituted amino groups (e.g. —NH₂, —NHR, —N(R)₂), and the protected derivatives thereof. In some embodiments, optional substituents are independently selected from alkyl, alkoxy, haloalkyl, cycloalkyl, halogen, —CN, —NH₂, —NH(CH₃), —N(CH₃)₂, —OH, —CO₂H, and —CO₂alkyl. In some embodiments, optional substituents are independently selected from fluoro, chloro, bromo, iodo, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic, saturated or unsaturated carbon atoms, excluding aromatic carbon atoms) includes oxo (═O).

The section headings used herein are for organizational purposes only and are not to be construed as limiting the subject matter described.

### EXAMPLES

These examples are provided for illustrative purposes only and not to limit the scope of the claims provided herein.

Preparations

The preparations set out below are for the synthesis of reagents that were not obtained from commercial sources and were employed for the preparation of compounds of formula I of the invention. All chiral compounds in the tables and schemes are racemic unless specified otherwise.

### Example 1: CRBN Fluorescence Polarization Assay with BODIPY-IMid Tracer

Promega assay was used to compare bifunctional ligands of the present invention to compete with IMiDs tracer binding to CRBN.

### Stock Solutions

46 uM CRBN (P85) DDB1 (P82)—Labelled P0071 180820

25 uM BODIPY-IMiD tracer (aliquots from Promega 160 uM stock solution)

Buffer: 100 mM Tris pH7.5, 150 mM NaCl, no DTT

Plates: PCR plate 96-well, Black 384-well

### Overview

500 nM CRBN-DDB1

30 nM BODIPY-IMiD

384-well plate 20 uL total volume

10 uL of CRBN-DDB1, BODIPY-IMiD added to 10 uL of ligand

### Protocol

A full 384-well assay plate was used to test bifunctional compounds with and without protein, Pomalidomide used as a control. Two 96-well PCR plates were prepared as follows:
1. Plate 1
   a. To wells A1-A11 on a 96-well PCR plate 100 uM bifunctional compound was added to 40 uL total volume
      i. 38 uL of buffer was added
      ii. 2 uL was added of 2 mM bifunctional or IMiD compound in DMSO or 2 uL of 0.2 mM for CC220 analogs
   b. To well A12 Pomalidomide was added as control
      i. 38 uL buffer was added
      ii. 2 uL of 2 mM Pomalidomide in DMSO was added
2. Plate 2
   a. To wells A1-A10 on a 96-well PCR plate 100 uM bifunctional compound was added to 40 uL total volume
      i. 38 uL of buffer was added
      ii. 2 uL of 2 mM bifunctional compound in DMSO was added
   b. To well A11 CC220 control was added
      i. 38 uL buffer added
      ii. 2 uL of 0.2 mM for CC220 was added
   c. To well A12 DMSO control was added
      i. Added buffer to 38 uL
      ii. Added 2 uL of DMSO
3. Prepared 7 mL of Buffer with 5% DMSO for serial dilution
   a. 26.67 uL to rows B-H on 96-well plate
4. Serial dilutions of samples in row A1 3-fold
   a. 13.3 uL of A1 added to 26.67 uL in A2
5. Added 10 uL to 384-well plate
   a. Added A1 from Plate 1 to wells A1 and A2 on 384-well plate (top half of 384-well plate)
   b. Added A1 from Plate 2 to wells I1 and I2 on 384-well plate (top half of 384-well plate)

A preparation was made of 500 nM CRBN-DDB1, 30 nM IMiD complex. To 4 mL of the prepared 500 nM CRBN-DDB1, 30 nM BODIPY-IMiD was added 47.6 uL of 46 uM CRBN-DDB1, 3.752 mL of Buffer (4 additions of 938 uL), 200 uL of DMSO, and 4.8 uL of 25 uM BODIPY IMiD stock. This incubated at room temperature for 10 minutes and then 10 uL was added to all wells on 384-well plate except wells M23-24, N23-24, O23-24, P23-24. Gain Control samples were then made consisting of 950 uL of Buffer, 50 uL of DMSO, 1.2 uL of 25 uM BODIPY IMiD stock, and 10 uL was added to wells M23-24, N23-24, O23-24, P23-24.

This was covered with foil and centrifuged for 30s at 500G and then incubated for 20 minutes and then read on Clariostar using method: FP>BODIPY_576/589; Filters needed:

i. Excitation 540-20

ii. Dichroic mirror: LP 566

Emission 590-20

The results are shown in Table 6.

TABLE 6

| Compound | | Result |
|---|---|---|
| Pomalidomide | | 1.57 |
| Lenalidomide | | 2.4 |
| 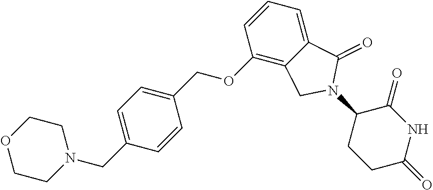 [SMILES: O=C1CC[C@@H](N2Cc3c(OCc4ccc(CN5CCOCC5)cc4)cccc3C2=O)C(=O)N1] | | 0.16 |
| 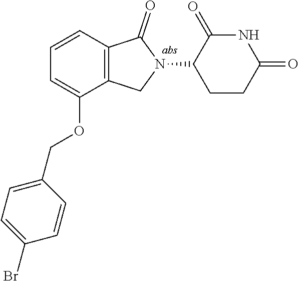 | (S)-3-(4-((4-bromobenzyl)oxy)-1- oxoisoindolin-2-yl)piperidine-2,6-dione | 0.25 |
| 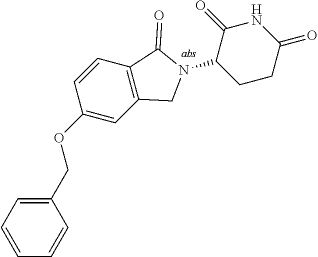 | (S)-3-(5-(benzyloxy)-1-oxoisoindolin-2- yl)piperidine-2,6-dione | 0.54 |
| 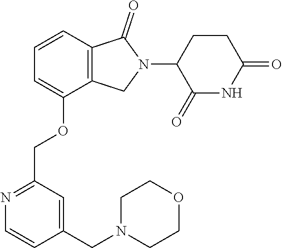 [SMILES: O=C1CCC(N2Cc3c(OCc4cc(CN5CCOCC5)ccn4)cccc3C2=O)C(=O)N1] | 3-(4-((4-(morpholinomethyl)pyridin-2- yl)methoxy)-1-oxoisoindolin-2- yl)piperidine-2,6-dione | 0.94 |
| 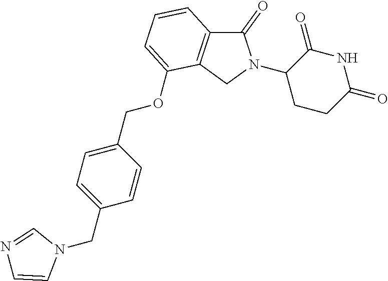 [SMILES: O=C1CCC(N2Cc3c(OCc4ccc(Cn5ccnc5)cc4)cccc3C2=O)C(=O)N1] | 3-(4-((4-((1H-imidazol-1- yl)methyl)benzyl)oxy)-1-oxoisoindolin-2- yl)piperidine-2,6-dione | 0.16 |
| 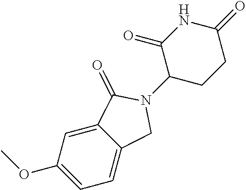 [SMILES: COc1ccc2c(c1)C(=O)N(C1CCC(=O)NC1=O)C2] | 3-(6-methoxy-1-oxoisoindolin-2- yl)piperidine-2,6-dione | 1.43 |
| 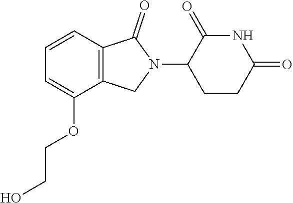 [SMILES: O=C1CCC(N2Cc3c(OCCO)cccc3C2=O)C(=O)N1] | 3-(4-(2-hydroxyethoxy)-1-oxoisoindolin-2- yl)piperidine-2,6-dione | 0.93 |
| 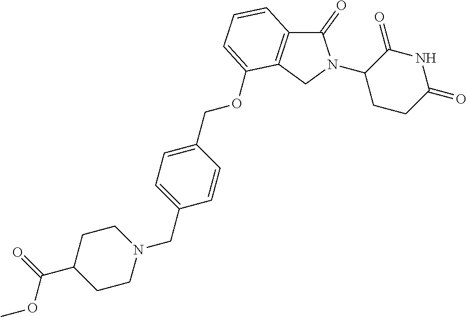 [SMILES: COC(=O)C1CCN(Cc2ccc(COc3cccc4c3CN(C3CCC(=O)NC3=O)C4=O)cc2)CC1] | methyl 1-(4-(((2-(2,6-dioxopiperidin-3-yl)- 1-oxoisoindolin-4- yl)oxy)methyl)benzyl)piperidine-4- carboxylate | 0.05 |
| 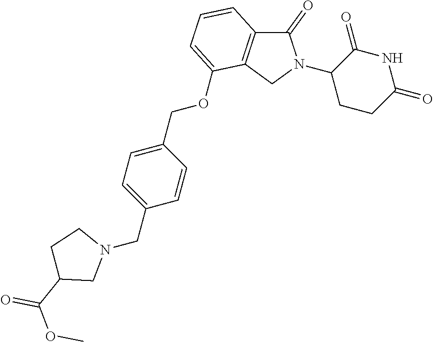 [SMILES: COC(=O)C1CCN(Cc2ccc(COc3cccc4c3CN(C3CCC(=O)NC3=O)C4=O)cc2)C1] | methyl 1-(4-(((2-(2,6-dioxopiperidin-3-yl)- 1-oxoisoindolin-4- yl)oxy)methyl)benzyl)pyrrolidine-3- carboxylate | 0.14 |
| 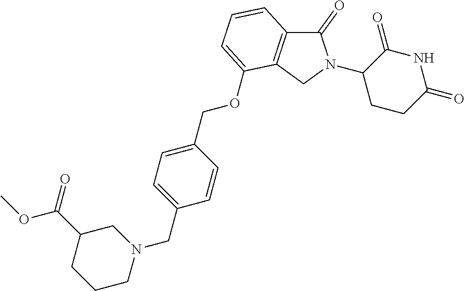 [SMILES: COC(=O)C1CCCN(Cc2ccc(COc3cccc4c3CN(C3CCC(=O)NC3=O)C4=O)cc2)C1] | methyl 1-(4-(((2-(2,6-dioxopiperidin-3-yl)- 1-oxoisoindolin-4- yl)oxy)methyl)benzyl)piperidine-3- carboxylate | 0.06 |
| 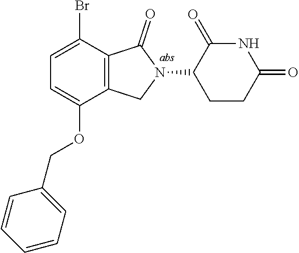 | (S)-3-(4-(benzyloxy)-7-bromo-1- oxoisoindolin-2-yl)piperidine-2,6-dione | 0.1 |

### Example 2

The table below illustrates the cereblon protein sequence.

| | | SEQ |
|---|---|---|
| | Sequence | ID NO: |
| Cereblon | MAGEGDQQDAAHNMGNHLPLLPAESEEEDEMEVED | 1 |
| UniProt- | QDSKEAKKPNIINFDISLPTSHTYLGADMEEFHGRTL | |
| Q96SW2 | HDDDSCQVIPVLPQVMMILIPGQTLPLQLFHPQEVSM | |
| (homo sapiens) | VRNLIQKDRTFAVLAYSNVQEREAQFGTTAEIYAYRE | |
| | EQDFGIEIVKVKAIGRQRFKVLELRTQSDGIQQAKVQI | |
| | LPECVLPSTMSAVQLESLNKCQIFPSKPVSREDQCSY | |
| | KWWQKYQKRKFHCANLTSWPRWLYSLYDAETLMD | |
| | RIKKQLREWDENLKDDSLPSNPIDFSYRVAACLPIDD | |
| | VLRIQLLKIGSAIQRLRCELDIMNKCTSLCCKQCQETE | |
| | ITTKNEIFSLSLCGPMAAYVNPHGYVHETLTVYKACN | |
| | LNLIGRPSTEHSWFPGYAWTVAQCKICASHIGWKFTA | |
| | TKKDMSPQKFWGLTRSALLPTIPDTEDEISPDKVILCL | |

What is claimed is:

1. A compound having the structure of Formula (I), 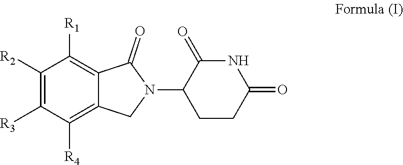
or pharmaceutically acceptable salt thereof, wherein
R¹, R², and R³ are each interpedently selected from hydrogen, halogen, halogenated alkyl, nitro, —COOH, —CONH₂, —CN, —N(R⁵)₂, —OR⁵, —C(═O)R⁵, —C(═O)N(R⁵)₂, —N(R⁵)C(═O)R⁵, —S(═O)₂R⁵, —N(R⁵)S(═O)₂R⁵, —S(═O)₂N(R⁵)₂, —(CH₂)₁₋₆₀R⁵, —O(CH₂)₁₋₆-A-R⁵, —O(CH₂)₁₋₆-A-R⁵, —O(CH₂)₁₋₆-A¹-(CH₂)₁₋₆-A²-C(O)OR⁵, substituted or unsubstituted C₁-C₆alkyl, substituted or unsubstituted C₃₋₈ cycloalkyl, substituted or unsubstituted 3-10 membered heterocyclyl, substituted or unsubstituted C₆₋₁₀ aryl, or substituted or unsubstituted 5-10 membered heteroaryl;
R⁴ is selected from hydrogen, halogen, halogenated alkyl, nitro, —COOH, —CONH₂, —CN, —N(C₁₋₆alkyl)₂, —OR⁵—, —C(═O)R⁵, —C(═O)N(R⁵)₂, —N(R⁵)C(═O)R⁵, —S(═O)₂R⁵, —N(R⁵)S(═O)₂R⁵, —S(═O)₂N(R⁵)₂, —(CH₂)₁₋₆OR⁵, —O(CH₂)₁₋₆-A-R⁵, —O(CH₂)₁₋₆-A-R⁵, —O(CH₂)₁₋₆-A¹-(CH₂)₁₋₆-A²-C(O)OR⁵, substituted or unsubstituted C₁-C₆alkyl, substituted or unsubstituted C₃₋₈ cycloalkyl, substituted or unsubstituted 3-10 membered heterocyclyl, substituted or unsubstituted C₆₋₁₀ aryl, or substituted or unsubstituted 5-10 membered heteroaryl;
each A¹ is independently a C₆₋₁₀ arylene or 5-10 membered heteroarylene,
each A² is independently a C₆₋₁₀ arylene, 4-10 membered heterocyclylene, or 4-10 membered heteroarylene, and
each R⁵ is independently H, halogen, substituted or unsubstituted C₁-C₆alkyl, substituted or unsubstituted C₁-C₆haloalkyl, substituted or unsubstituted aryl, or substituted or unsubstituted heteroaryl.

2. The compound of claim 1, wherein R¹ hydrogen, halogen, halogenated alkyl, CN, C₁₋₆ alkyl, or alkoxyl.

3. The compound of claim 1 or 2, wherein R² is halogen, halogenated alkyl, CN, C₁₋₆ alkyl, or alkoxyl.

4. The compound of claim 3, wherein R¹, R3, and R⁴ are hydrogen.

5. The compound of any one claims 1-3, wherein R⁴ is —O(CH₂)₁₋₆-A¹-R⁵, —O(CH₂)₁₋₆A¹-(CH₂)₁₋₆-A²-C(O)OR⁵.

6. The compound of any one claims 1-5, wherein R⁵ is H or C₁₋₆ alkyl.

7. The compound of claim 5, wherein R¹, R², and R³ are not all hydrogen.

8. The compound of claim 5, wherein at least one of R¹, R², and R is hydrogen but not all R¹, R², and R³ are hydrogen.

9. The compound of claim 5, wherein A¹ is phenylene or pyrodylene.

10. The compound of claim 5, wherein A² is

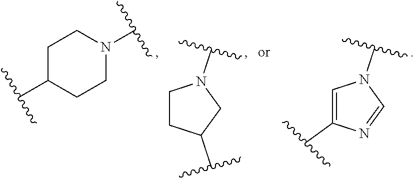 [SMILES: *C1CCN(*)CC1 ; *C1CCN(*)C1 ; *c1cn(*)cn1]

11. The compound of claim 1 or 2, wherein R² is halogen, halogenated alkyl, or PG alkoxyl, and R¹, R³, and R⁴ are hydrogen.

12. The compound of any one of claims 1-3, wherein R³ or R⁴ is OCH₂phenyl.

13. The compound of any one of claims 1-3, wherein only one of R³ or R⁴ is OCH₂phenyl.

14. The compound of any one of claims 1-3, wherein R³ or R⁴ is —NHC(O)C₁₋₆alkyl.

15. The compound of any one of claims 1-3, wherein only one of R³ or R⁴ is —NHC(O)C₁₋₆alkyl.

16. The compound of any one of claims 1-3, wherein R³ and R⁴ are independently alkoxyl, N(C₁₋₆alkyl)₂, COOH, or CONH₂, SO₂CH₃, phenyl, halogen, or cycloalkyl.

17. The compound of claim 1, wherein R¹ is —NHC(O)C₁₋₆alkyl.

18. The compound of claim 1, wherein R¹, R², R³, and R⁴ are not all hydrogen.

19. The compound of claim 18, wherein at least one of R¹, R², R³, and R⁴ is hydrogen.

20. The compound of claim 1, wherein R¹ or R⁴ is not NH₂.

21. The compound of claim 1, wherein having the structure of Formula (IIA) or (IIB)

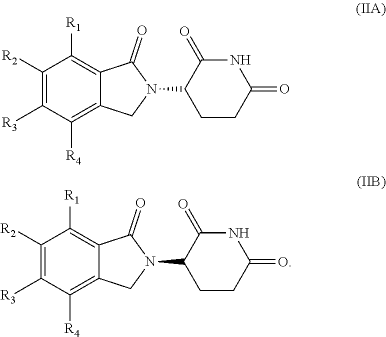

22. The compound of claim 1, wherein having the structure of Formula (IIA)

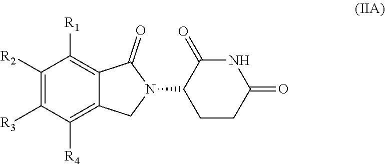

23. The compound of claim 1, wherein R¹ is H, substituted or unsubstituted C₁-C₆alkyl, or substituted or unsubstituted C₁-C₆aminoalkyl.

24. The compound of claim 1, wherein R² is H, unsubstituted C₁-C₆alkyl; C₁-C₆alkyl substituted with one or more substituents selected from C₆₋₁₀ aryl, C₃₋₈ cycloalkyl, 3-10 membered heterocyclyl, 5-10 membered heteroaryl, halogen, and amine; C₁₋₆ alkylene-C₆₋₁₀ aryl substituted with one or more substituents selected from C₁₋₆ alkyl, alkoxyl, halogen, and amine; and substituted or unsubstituted C₁-C₆aminoalkyl.

25. The compound of claim 1, wherein R² is H, substituted or unsubstituted C₁-C₆alkyl, or substituted or unsubstituted C₁-C₆aminoalkyl.

26. The compound of claim 1, wherein R³ is H, unsubstituted C₁-C₆alkyl; C₁-C₆alkyl substituted with one or more substituents selected from C₆₋₁₀ aryl, C₃₋₈ cycloalkyl, 3-10 membered heterocyclyl, 5-10 membered heteroaryl, halogen, and amine; C₁₋₆ alkylene-C₆₋₁₀ aryl substituted with one or more substituents selected from C₁₋₆ alkyl, alkoxyl, halogen, and amine; and substituted or unsubstituted C₁-C₆aminoalkyl.

27. The compound of claim 1, wherein R³ is H, substituted or unsubstituted C₁-C₆alkyl, or substituted or unsubstituted C₁-C₆aminoalkyl.

28. The compound of claim 1, wherein R⁴ is H, unsubstituted C₁-C₆alkyl; C₁-C₆alkyl substituted with one or more substituents selected from C₆₋₁₀ aryl, C₃₋₈ cycloalkyl, 3-10 membered heterocyclyl, 5-10 membered heteroaryl, halogen, and amine; C₁₋₆ alkylene-C₆₋₁₀ aryl substituted with one or more substituents selected from C₁₋₆ alkyl, alkoxyl, halogen, and amine; and substituted or unsubstituted C₁-C₆aminoalkyl.

29. The compound of claim 1, wherein R⁴ is H, substituted or unsubstituted C₁-C₆alkyl, or substituted or unsubstituted C₁-C₆aminoalkyl.

30. The compound of claim 1, wherein R⁵ is H, unsubstituted C₁-C₆alkyl; C₁-C₆alkyl substituted with one or more substituents selected from C₆₋₁₀ aryl, C₃₋₈ cycloalkyl, 3-10 membered heterocyclyl, 5-10 membered heteroaryl, halogen, and amine; C₁₋₆ alkylene-C₆₋₁₀ aryl substituted with one or more substituents selected from C₁₋₆ alkyl, alkoxyl, halogen, and amine; and substituted or unsubstituted C₁-C₆aminoalkyl.

31. The compound of claim 1, wherein R⁵ is H, substituted or unsubstituted C₁-C₆alkyl, or substituted or unsubstituted C₁-C₆aminoalkyl.

32. The compound of claim 1, wherein A¹ is a phenylene.

33. The compound of claim 1, wherein A¹ is a pyrodylene.

34. The compound of claim 1, wherein the compound of Formula (I) has a structure selected from Table 1-5.

35. The compound of claim 1, wherein the compound of Formula (I) is selected from the group consisting of (R)-3-(6-bromo-1-oxoisoindolin-2-yl)piperidine-2,6-dione, 3-(1-oxo-6-(trifluoromethyl)isoindolin-2-yl)piperidine-2,6-dione, and 3-(6-methoxy-1-oxoisoindolin-2-yl)piperidine-2,6-dione.

36. A pharmaceutical composition comprising the compound of any one of claims 1-35 and a pharmaceutically acceptable excipient.

37. A bifunctional degrader comprising a targeting ligand, linker, and a compound selected from Tables 1-5.

38. A bifunctional degrader comprising a targeting ligand, linker, and the compound of any one of claims 1-35.

39. A method of treating a disease or disorder in a subject, the method comprising administering a composition comprising an effective amount of the compound of any one of claims 1-35 and a pharmaceutically acceptable carrier, additive, and/or excipient to a subject in need thereof, wherein the compound is effective in treating or ameliorating at least one symptom of the disease or disorder.

40. A method of treating a disease or disorder in a subject, the method comprising administering a composition comprising an effective amount of at least one compound from Tables 1-5 and a pharmaceutically acceptable carrier, additive, and/or excipient to a subject in need thereof, wherein the compound is effective in treating or ameliorating at least one symptom of the disease or disorder.

41. A method of modulating a biological activity of cereblon, comprises: contacting the cereblon with an effective amount of the compound of any one of claims 1-35.

42. The method of claim 1, wherein the biological activity is a tumoricidal activity.

43. The method of claim 1, wherein the biological activity is an apoptosis activity.

44. The method of claim 1, wherein the biological activity is peripheral blood mononuclear cell (PBMC) viability.
